(19) United States
(12) Patent Application Publication
(10) Pub. No.: US 2015/0344859 A1
(43) Pub. Date: Dec. 3, 2015

(54) COMPOSITION FOR USE IN MYCOBACTERIA THERAPY

(71) Applicant: Lysando AG, Triesenberg (LI)

(72) Inventor: Stefan Miller, Regensburg (DE)

(21) Appl. No.: 14/411,636

(22) PCT Filed: Jul. 1, 2013
(86) PCT No.: PCT/EP2013/063846

§ 371 Date: Dec. 29, 2014

(30) Foreign Application Priority Data
Jun. 29, 2012 (EP) 12174467.6

(51) Int. Cl. C12N 9/36; A23K 1/17; A23L 1/30

(52) U.S. Cl. CPC C12N 9/2462; A23L 1/3014; A23K 1/17; A61K 38/00

(57) ABSTRACT

The present invention relates to a composition having the activity of degrading the cell wall of a Mycobacterium species comprising: (a) a first fusion protein comprising (i) a first endolysin or a first domain, both having a first enzymatic activity; (ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (iii) a protein transduction domain (PTD) being at the N- or C-terminus of the first fusion protein, wherein the PTD is having the characteristic to deliver a cargo from the extracellular to the intracellular space of a cell; and (b) a second fusion protein comprising (i) a second endolysin or a second domain, both having a second enzymatic activity; (ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (iii) a protein transduction domain (PTD) being at the N- or C-terminus of the second fusion protein, wherein the PTD is having the characteristic to deliver a cargo from the extracellular to the intracellular space of a cell. Moreover, the present invention relates in particular to said composition for use as a medicament, a disinfectant, a feed additive, or a food additive as well as the use of said composition in the treatment or prevention of diseases caused by an infection with a Mycobacterium species.

The present invention relates to a composition having the activity of degrading the cell wall of a Mycobacterium species comprising: (a) a first fusion protein including (i) a first endolysin or a first domain, both having a first enzymatic activity, the enzymatic activity being at least one or more of the following: N-acetyl-b-D-muramidase (lysozyme, lytic transglycosylase), N-acetyl-b-D-glucosaminidase, N-acetylmuramoyl-L-alanine amidase, L-alanoyl-D-glutamate (LD) endopeptidase, c-D-glutamyl-meso-diaminopimelic acid (DL) peptidase, L-alanyl-D-iso-glutaminyl-meso-diaminopimelic acid (D-Ala-m-DAP) (DD) endopeptidase, or m-DAP-m-DAP (LD) endopeptidase, (ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (iii) a protein transduction domain (PTD) being at the N- or C-terminus of the first fusion protein, wherein the PTD is having the characteristic to deliver a cargo from the extracellular to the intracellular space of a cell; and (b) a second fusion protein including (i) a second endolysin or a second domain, both having a second enzymatic activity, the enzymatic activity being at least one or more of the following: lipolytic activity, cutinase, mycolarabinogalactanesterase, or alpha/beta hydrolase; (ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (iii) a protein transduction domain (PTD) being at the N- or C-terminus of the second fusion protein, wherein the PTD is having the characteristic to deliver a cargo from the extracellular to the intracellular space of a cell. Moreover, the present invention relates in particular to said composition for use as a medicament, a disinfectant, a feed additive, or a food additive as well as the use of said composition in the treatment or prevention of diseases caused by an infection with a Mycobacterium species.

Mycobacteria are classified as Gram positive bacteria. In comparison to most of the Gram-positive bacteria however, the structure of the cell wall of mycobacteria is different in their composition. The complex structure of the cell wall of mycobacteria consists of a mycolic acid-rich outer membrane which is covalently linked to the arabinogalactan-petidoglycan complex (Hoffmann et al Proc Natl Acad Sci USA.; 105(10):3963-7; 2008; Zuber et al., J Bacteriol.; 190(16):5672-80; 2008). The mycolic acids are alpha-alkyl, beta-hydroxy C₆₀₋₉₀ fatty acids. The distinct composition of the mycolic acids is dependent on the Mycobacterium species including short saturated alpha, C₂₀₋₂₅, and a longer meromycolate chain, the beta-hydroxy branch C₆₀, comprising doublebonds, cyclopropane rings and oxygenated groups. The outer membrane is linked with esterification to the terminal pentaarabinofuranosyl components of arabinogalactan (Payne et al., Molecular Microbiology, 73(3), 2009). The arabinogalactan is covalently linked to peptidoglycan. This covalently linked complex is known as mycolyl-arabinogalactan peptidoglycan (mAGP). This mAGP is known as the cell wall core and builds a stable scaffolding to anchor the outer non-covalently associated lipid and glycoplipids including trehalose 6,6′-dimycolate (TDM or cord factor) (Gil et al., Microbiology, 156, 2010). TDM is a secreted molecule which is important for the pathogenesis of mycobacteria (Brennan, Tuberculosis (Edinb); 83(1-3): 91-7, 2003). The cell surface of mycobacteria has the characteristics of a highly hydrophobicity and fastness in view of acids due to the special structure of mAGP and TDM in combination with trehalose 6′-monomycolate. These special properties are leading to the fact that mycobacteria are resistant to dehydration and possess a natural impermeability to nutrients and antibacterial drugs (Gil et al., Microbiology, 156, 2010).

Mycobacterium tuberculosis is the cause for the tuberculosis, an infectious disease which typically affects the lungs. Tuberculosis is a health and life threatening disease. In 2009, 9.4 million new cases of tuberculosis and 1.7 million deaths are counted (Global Tuberculosis Control WHO Report 2010. World Health Organization; Geneva: 2010). Mycobacterium tuberculosis is spread as a primarily respiratory pathogen. Patients with an active infection can transmit the infection by coughing. A major part of infected human patients are not able to eliminate the bacteria completely. This results in the so called “latent” stage, defining a status, wherein the patient is still infected, but does not show any symptoms of the disease. This latent stage however can change in some patients due to a reactivation of the infection resulting in an active stage of tuberculosis. Typically, an infection with mycobacterium tuberculosis starts with the inhalation of the bacteria, followed by the presentation by antigen-presenting immune cells, such as macrophages or dendritic cells, in the airway. Infected macrophages include mycobacteria in intracellular vesicles. However, these vesicles are not accessible for a fusion with lysosomes, which would result in a killing of the mycobacteria. After activation of the infected macrophages with a specific T_{H}1-cell, a lysosomal fusion occurs. Further to this first infection step, infected macrophages recruit uninfected macrophages. Thereby a so called granuloma is formed. The structure of such a granuloma, which is also called caseous granuloma because of the “cheese-like” look, comprises macrophages surrounding a necrotic area with adjacent of B and T cells.

Mycobacteria in general can be classified into several major groups for purpose of diagnosis and treatment: M. tuberculosis complex, which can cause tuberculosis: M. tuberculosis, M. bovis, M. africanum, and M. micron; M. leprae, which causes Hansen's disease or leprosy; Nontuberculous mycobacteria (NTM) define all the other mycobacteria, which can cause pulmonary disease resembling tuberculosis, lymphadenitis, skin disease, or disseminated disease. Mycobacteria not only cause human infections but also animal infections as well, e.g. Mycobacterium avium, Mycobacterium avium subsp. paratuberculosis, Mycobacerium bovis.

Mycobacteriophages are a subgroup of bacteriophages, which are bacterial viruses, which target mycobacterial hosts. In view of the special structure and composition of the cell wall of mycobacteria, it is necessary for the mycobacteriophages to degrade the peptidoglycan layer and further to lyse the mycolic acid-rich outer membrane attached to the mAGP complex.

Various types of agents having bactericidal or bacteriostatic activity are known, e.g. antibiotics, endolysins, antimicrobial peptides such as defensins. Further, phages are known to exert bactericidal activity as well. Increasingly microbial resistance to antibiotics, however, is creating difficulties in treating more and more infections caused by bacteria.

Endolysins are peptidoglycan hydrolases encoded by bacteriophages (or bacterial viruses). They are synthesized during late gene expression in the lytic cycle of phage multiplication and mediate the release of progeny virions from infected cells through degradation of the bacterial peptidoglycan. They are either β(1,4)-glycosylases, transglycosylases, amidases or endopeptidases. Antimicrobial application of endolysins was already suggested in 1991 by Gasson (GB2243611). Although the killing capacity of endolysins has been known for a long time, the use of these enzymes as antibacterials was ignored due to the success and dominance of antibiotics. Only after the appearance of multiple antibiotic resistant bacteria this concept of combating human pathogens with endolysins received interest. A compelling need to develop totally new classes of antibacterial agents emerged and endolysins used as ‘enzybiotics’—a hybrid term of ‘enzymes’ and ‘antibiotics’—seem to meet this need. In 2001, Fischetti and coworkers demonstrated for the first time the therapeutic potential of bacteriophage Cl endolysin towards group A streptococci (Nelson et al., Proc Natl Acad Sci USA. 98(7):4107-12, 2001). Since then many publications have established endolysins as an attractive and complementary alternative to control bacterial infections, particularly by Gram positive bacteria. Subsequently different endolysins against other Gram positive pathogens such as Streptococcus pneumoniae (Loeffler et al., Science; 294(5549):2170-2, 2001), Bacillus anthracis (Schuch et al., Nature.; 418(6900):884-9, 2002), S. agalactiae (Cheng et al., Antimicrob Agents Chemother; 49(1):111-7, 2005) and Staphylococcus aureus (Rashel et al., J Infect Dis; 196(8):1237-47, 2007) have proven their efficacy as enzybiotics.

Distinct endolysins have been identified in mycobacteriophages (Payne and Hatfull, Plos ONE, 7(3), 2012; Payne et al., Mol Microbiol, 73(3), 2009). These particular endolysins are able to break down the mycobacterial cell wall characterized by the mycol-rich mycobacterial outer membrane attached to an arabinogalactan layer which is in turn linked to the peptidoglycan. These particular phage endolysins can be assigned to two groups, (i) enzymes that cleave the peptidoglycan, and (ii) enzymes that cleave the mycolic acid and arabinogalactan layer.

Antimicrobial peptides (AMPs) represent an important component of the innate immunity against infections against bacteria. Several antimicrobial peptides have been identified which possess an effect against mycobacteria. These antimicrobial peptides are involved not only in the killing of mycobacteria but also in the modulation of the immune defense in form of the secretion of cytokines and chemokines (Shin and Jo, Immune Network, 11(5), 2011).

Antimicrobial peptides (AMPs) represent a wide range of short, cationic or amphipathic, gene encoded peptide antibiotics that can be found in virtually every organism. Different AMPs display different properties, and many peptides in this class are being intensively researched not only as antibiotics, but also as templates for cell penetrating peptides. Despite sharing a few common features (e.g., cationicity, amphipathicity and short size), AMP sequences vary greatly, and at least four structural groups (α-helical, (β-sheet, extended and looped) have been proposed to accommodate the diversity of the observed AMP conformations. Likewise, several modes of action as antibiotics have been proposed, and it was shown e.g. that the primary target of many of these peptides is the cell membrane whereas for other peptides the primary target is cytoplasmic invasion and disruption of core metabolic functions. AMPs may become concentrated enough to exhibit cooperative activity despite the absence of specific target binding; for example, by forming a pore in the membrane, as is the case for most AMPs. However, this phenomenon has only been observed in model phospholipid bilayers, and in some cases, AMP concentrations in the membrane that were as high as one peptide molecule per six phospholipid molecules were required for these events to occur. These concentrations are close to, if not at, full membrane saturation. As the minimum inhibitory concentration (MIC) for AMPs are typically in the low micromolar range, scepticism has understandably arisen regarding the relevance of these thresholds and their importance in vivo (Melo et al., Nature reviews, Microbiology, 2009, 245).

Cathelicidins are a family of AMPs which are derived from leukocytes and epithelial cells. Currently, the only identified human cathelicidin is hCAP-18/LL-37 Immunstimulatory effects have been reported for cathelicidins (Shin and Jo, Immune Network, 11(5), 2011).

Defensins are a large family of small, cationic or amphipathic, cysteine- and arginine-rich antimicrobial peptides, found in both vertebrates and invertebrates. Defensins are divided into five groups according to the spacing pattern of cysteines: plant, invertebrate, α-, ρ-, and θ-defensins. The latter three are mostly found in mammals. α-defensins are proteins found in neutrophils and intestinal epithelia. β-defensins are the most widely distributed and are secreted by leukocytes and epithelial cells of many kinds. θ-defensins have been rarely found so far e.g. in leukocytes of rhesus macaques. Defensins are active against bacteria, fungi and many enveloped and nonenveloped viruses. However, the concentrations needed for efficient killing of bacteria are mostly high, i.e. in the μ-molar range. Activity of many peptides may be limited in presence of physiological salt conditions, divalent cations and serum. Depending on the content of hydrophobic amino acid residues defensins also show haemolytic activity.

Hepcidin is a cationic amphipathic bactericidal peptide which is primarily produced in the liver. The expression of Hepcidin is induced during infectious and inflammatory conditions. Crucially, Hepcidin is expressed in macrophages after infection with intracellular pathogens Mycobacterium avium and Mycobacterium tuberculosis. Further, hepcidin causes damage to Mycobacterium tuberculosis and thus exerts immediate antimycobacterial activity (Shin and Jo, Immune Network, 11(5), 2011).

Mycobacteria with its special structure of the cell wall and the infection procedure which results in the intracellular survival of the mycobacteria within macrophages represent challenges for an effective treatment and therapy of mycobacterial infections. There are currently difficulties existing to target mycobacteria which are residing and replicating intracellularly, e.g. mycobacteria which are surviving within infected host cells, such as macrophages.

Thus, there is a need for new antimycobacterial agents.

This object is solved by the subject-matter defined in the claims.

The following figures describe the invention.

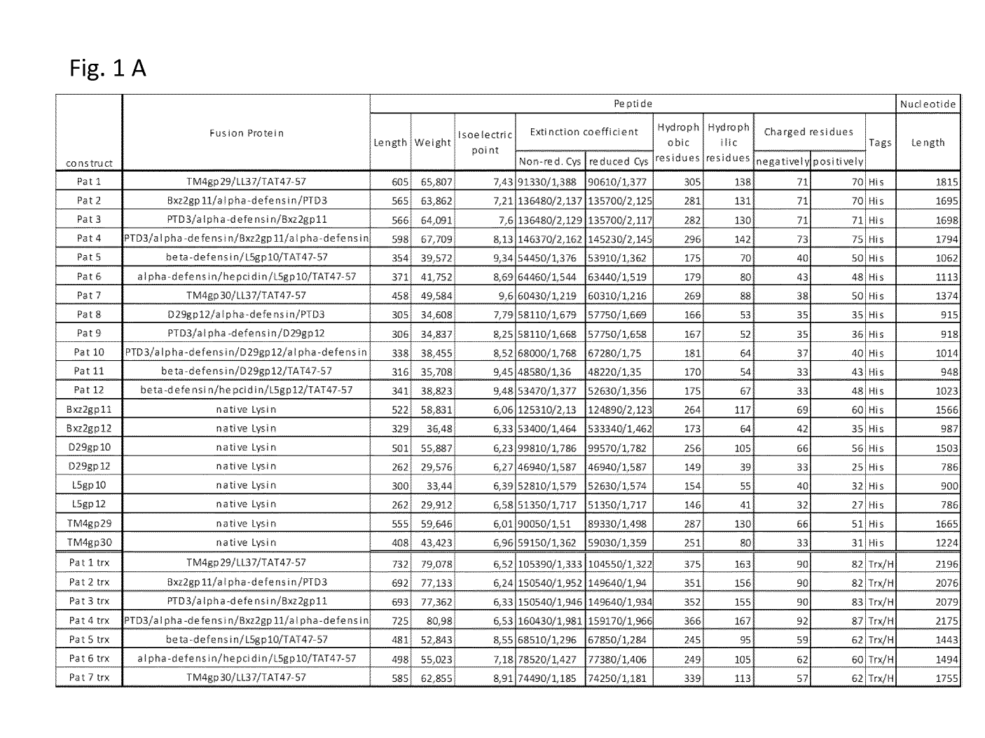

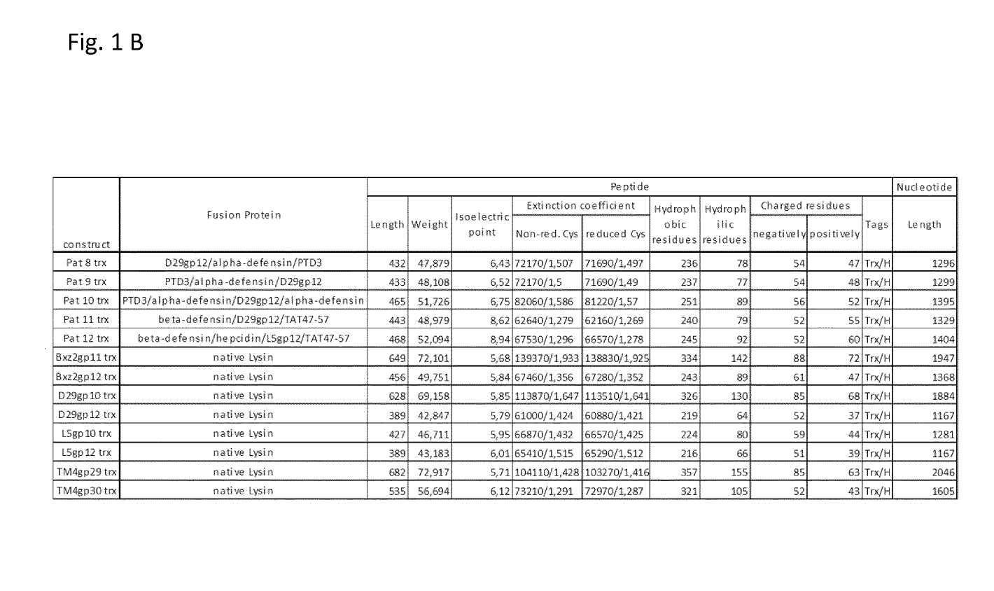

FIGS. 1 A and B provides an overview of distinct parameters of the first and second fusion proteins of the invention.

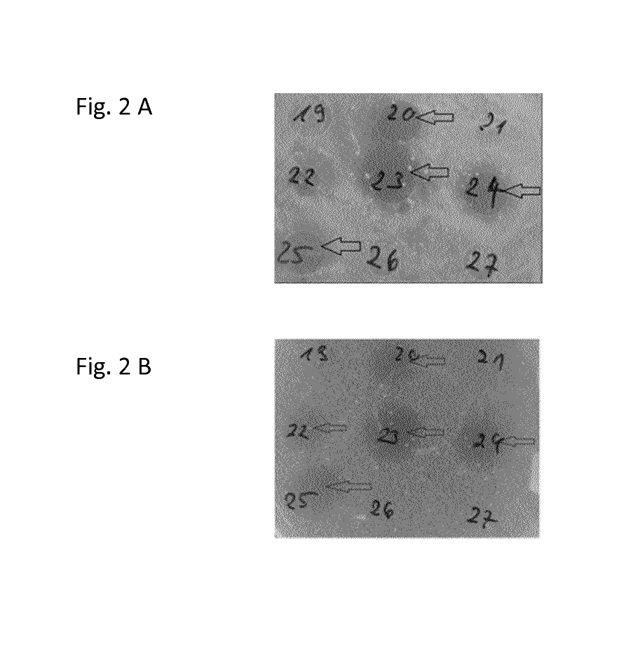

FIGS. 2 A and B is a picture of the spot test on agarose plates with mycobacerial lawn (FIG. 2 A) and an agarose-overlay spot test (FIG. 2 B).

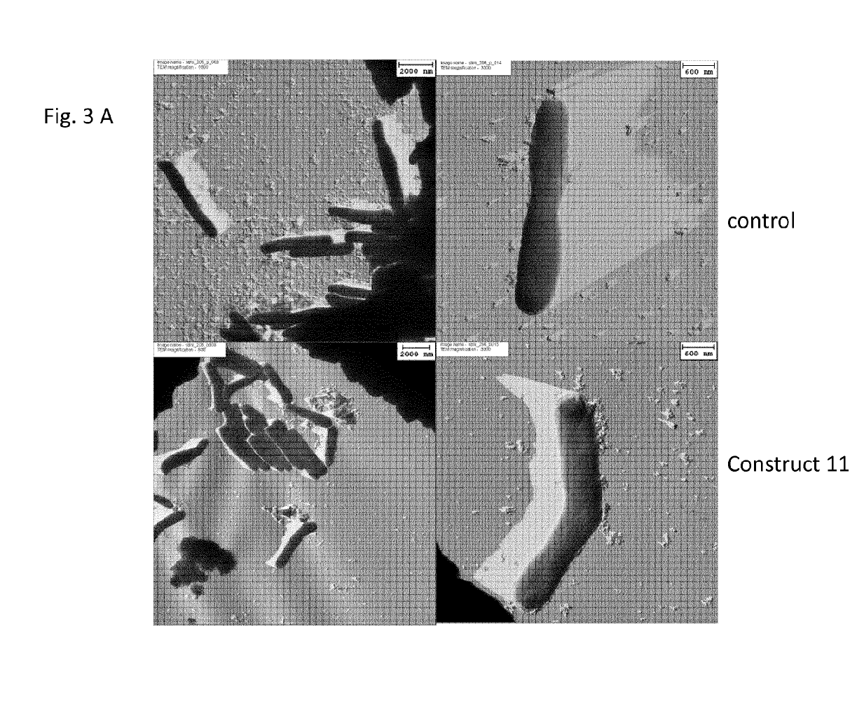

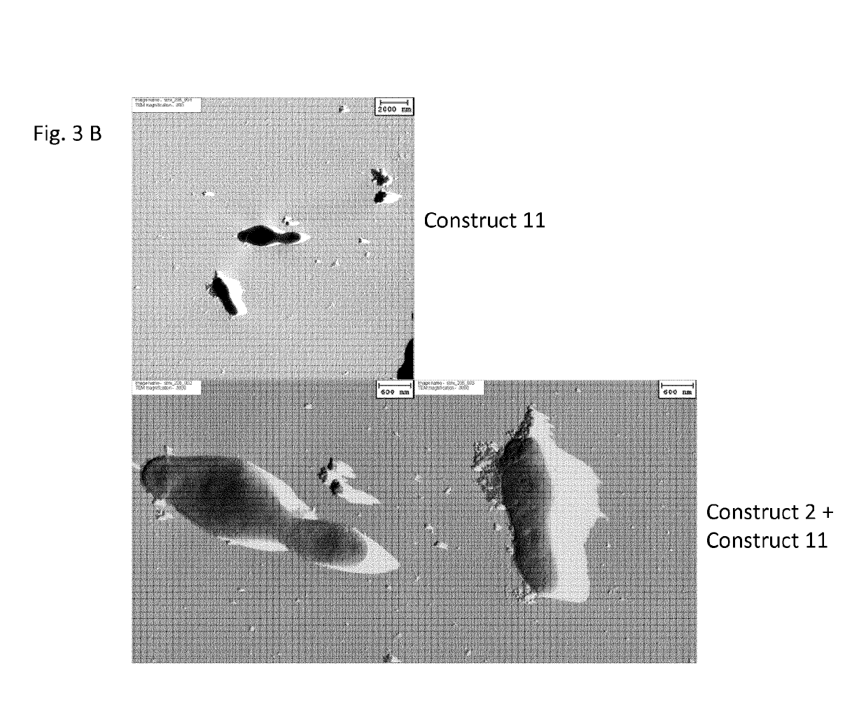

FIGS. 3 A and B is an electron microscopy picture of mycobacteria cells. Shown are pictures of untreated mycobacteria (FIG. 3 A upper row), treated only with construct 11 (FIG. 3 A lower row, and FIG. 3 B upper picture), and treated with first and second fusion protein of construct 2 and construct 11 according to the invention (FIG. 3 B lower row).

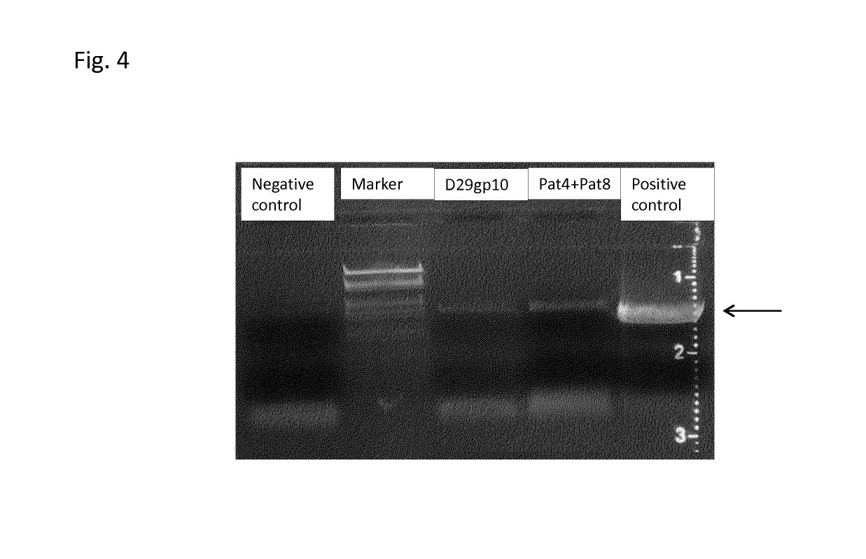

FIG. 4 shows a picture of an agarose gel electrophoresis.

The term “protein” as used herein refers to a linear polymer of amino acid residues linked by peptide bonds in a specific sequence. The amino-acid residues of a protein may be modified by e.g. covalent attachments of various groups such as carbohydrates and phosphate. Other substances may be more loosely associated with the protein, such as heme or lipid, giving rise to the conjugated proteins which are also comprised by the term “protein” as used herein. The protein may be folded in different ways. The various ways in which the protein fold have been elucidated, are in particular with regard to the presence of alpha helices and beta-pleated sheets. The term “protein” as used herein refers to all four classes of proteins being all-alpha, all-beta, alpha/beta and alpha plus beta. Moreover, the term “protein” refers to a complex, wherein the complex refers to a homomer.

The term “fusion protein” as used herein refers to an expression product resulting from the fusion of different nucleic acid sequences. Such a protein may be produced, e.g., in recombinant DNA expression systems. Moreover, the term “fusion protein” as used herein refers to a fusion of a first amino acid sequence having an enzymatic activity, e.g. an endolysin, with a second and a third amino acid sequence. The second amino acid sequence is preferably a peptide stretch, in particular selected from the group consisting of cationic, polycationic, hydrophobic, amphipathic peptides, and antimicrobial peptides. A third amino acid sequence is a protein transduction domain. Preferably, said second and third amino acid sequence is foreign to and not substantially homologous with any domain of the first amino acid sequence. Moreover, the fusion proteins of the present invention also refer to an expression product resulting from the fusion of at least three nucleic acid sequences.

The term “peptide stretch” as used herein refers to any kind of peptide linked to a protein such as an endolysin. In particular the term “peptide stretch” as used herein refers to a peptide stretch selected from the group consisting of cationic, polycationic, hydrophobic, amphipathic peptides, and antimicrobial peptides (AMP), in particular synthetic amphipathic peptide, synthetic cationic peptide, synthetic polycationic peptide, synthetic hydrophobic peptide, synthetic antimicrobial peptide (AMP) or naturally occurring AMP. In the context of the present invention, AMP are understood as peptides, which provide antimycobacterial activity.

However, a peptide stretch in the meaning of the present invention does not refer to His-tags, preferably His₅-tags, His₆-tags, His₇-tags, His₈-tags, His₉-tags, His₁₀-tags, His₁₁-tags, His₁₂-tags, His₁₆-tags and His₂₀-tags, Strep-tags, Avi-tags, Myc-tags, Gst-tags, JS-tags, cystein-tags, FLAG-tags or other tags known in the art, thioredoxin or maltose binding proteins (MBP). The term “tag” in contrast to the term “peptide stretch” as used herein refers to a peptide which can be useful to facilitate expression and/or affinity purification of a polypeptide, to immobilize a polypeptide to a surface or to serve as a marker or a label moiety for detection of a polypeptide e.g. by antibody binding in different ELISA assay formats as long as the function making the tag useful for one of the above listed facilitation is not caused by the positively charge of said peptide. However, the His₆-tag may, depending on the respective pH, also be positively charged, but is used as affinity purification tool as it binds to immobilized divalent cations and is not used as a peptide stretch according to the present invention.

The term “peptide” as used herein refers to short polypeptides consisting of from about 2 to about 100 amino acid residues, more preferably from about 4 to about 50 amino acid residues, more preferably from about 5 to about 30 amino acid residues, wherein the amino group of one amino acid residue is linked to the carboxyl group of another amino acid residue by a peptide bond. A peptide may have a specific function. A peptide can be a naturally occurring peptide or a synthetically designed and produced peptide. The peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). Preferred naturally occurring peptides are e.g. antimicrobial peptides and defensins. Preferred synthetically produced peptides are e.g. polycationic, amphipathic or hydrophobic peptides. A peptide in the meaning of the present invention does not refer to His-tags, Strep-tags, thioredoxin or maltose binding proteins (MBP) or the like, which are used to purify or locate proteins.

The term “enzymatic activity” as used herein refers to the effect exerted by one or more enzyme(s) or enzyme like substance(s). An enzymatic activity refers in particular to the effects which are exerted by endolysins. The term “enzymatic activity” refers further in particular to the effect of distinct group of enzyme or enzymatic substances which are having the activity of degrading the cell wall of a Mycobacterium species. A group of these enzymes with this distinct characteristics are named as Lysin A (LysA), of the peptidoglycan-cleavage group, which are known or are proposed to cleave (Payne and Hatfull, Plos ONE, 7(3), 2012; Payne et al., Mol Microbiol, 73(3), 2009):
N-acetyl-b-D-muramidase (lysozyme, lytic transglycosylase);
N-acetyl-b-D-glucosaminidase;
N-acetylmuramoyl-L-alanine amidase;
L-alanoyl-D-glutamate (LD) endopeptidase;
c-D-glutamyl-meso-diaminopimelic acid (DL) peptidase;
D-Ala-m-DAP (DD) endopeptidase; and
m-DAP-m-DAP (LD) endopeptidase.

A further group of enzymes are named as Lysin B (LysB). These enzymes hydrolyze the linkage of the mycolic acids to the peptidoglycan-arabinogalactan complex and comprise at least the following or other lipolytic activities:
Esterase (mycolarabinogalactanesterase)
Cutinase
α/β hydrolase

LysB like proteins are described e.g. in Mycobacteriophage Lysin B is a novel mycolylarabinogalactan esterase Kimberly Payne, Qingan Sun, James Sacchettini, Graham F. Hatfull Mol Microbiol. 2009 August; 73(3): 367-381; Mycobacteriophage Ms6 LysB specifically targets the outer membrane of Mycobacterium smegmatis Filipa Gil, Anna E. Grzegorzewicz, Maria João Catalão, João Vital, Michael R. McNeil, Madalena Pimentel Microbiology. 2010 May; 156(Pt 5): 1497-1504.

A person skilled in the art is able to identify an enzymatic activity as mentioned above with applying a suitable test setting for the distinct enzyme or enzymatic activity.

The term “endolysin” as used herein refers to an enzyme which is a peptidoglycan hydrolase naturally encoded by bacteriophages or bacterial viruses and which is suitable to hydrolyse bacterial cell walls. According to the present invention “endolysins” may derive from mycobacteriophages. Thus, “endolysins” are in particular enzymes such as Lysin A, LysA, or Lysin A like enzymes or Lysin B, LysB, or Lys B like enzymes. “Endolysins” comprise at least one “enzymatically active domain” (EAD) having at least one or more of the following activities: N-acetyl-b-D-muramidase (lysozyme, lytic transglycosylase), N-acetyl-b-D-glucosaminidase, N-acetyl-muramoyl-L-alanine-amidase (amidase) peptidase, L-alanoyl-D-glutamate (LD) endopeptidase, L-alanyl-D-iso-glutaminyl-meso-diaminopimelic acid (D-Ala-m-DAP) (DD) endopeptidase, or m-DAP-m-DAP (LD) endopeptidase. Furthermore, the EAD is having at least one or more of the following activities: lipolytic activity, cutinase, mycolarabinogalactanesterase, or alpha/beta hydrolase. In addition, the endolysins may contain also regions which are enzymatically inactive, and bind to the cell wall of the host bacteria, the so-called CBDs (cell wall binding domains). The endolysin may contain two or more CBDs. Generally, the cell wall binding domain is able to bind different components on the surface of bacteria. Preferably, the cell wall binding domain is a peptidoglycan binding domain and binds to the bacteria's peptidoglycan structure. The different domains of an endolysin can be connected by a domain linker.

The term “domain linker” as used herein refers to an amino acid sequence functioning to connect single protein domains with one another. As a rule domain linkers form no or only few regular secondary structure like α-helices or β-sheets and can occupy different conformations with the respective structural context. Methods to detect domain linker and properties of linker sequences are well known in the art as e.g. described in Bae et al., 2005, Bioinformatics, 21, 2264-2270 or George & Heringa, 2003, Protein Engineering, 15, 871-879.

The term “protein transduction domain” (PTD) or the term “cell penetrating peptides” (CPP) refers to an amino acid sequence functioning to deliver a cargo from the extracellular to the intracellular space of a cell. The transportation mediated by the PTD to deliver a cargo from the extracellular to the intracellular space involves a three step process, including first, binding of the PTD to the cellular membrane; second, stimulation of cellular uptake by endocytosis; and third, escape of cargo into the cytoplasm (Van den Berg and Dowdy, Current Opinion in Biotechnology, 22, 2011). PTDs may be cationic. The term “cargo” in the context of the present invention refers to a substance which is transported from the outside to the inside of a cell. The term “cargo” in the context of the present invention refers in particular to peptides, such as AMPs, enzymes, such as endolysins, dyes, such as fluorescent dyes like fluorescein. A person skilled in the art is able to identify amino acid sequences which are PTDs e.g. in form of an experimental setting wherein it is foreseen to use an amino acid sequence which is supposed to be a PTD together with a dye, such as a fluorescent dye, as a cargo. Using e.g. fluorescent microscopy or Fluorescent-activated cell sorting (FACS) analysis, allows assessing whether the putative PTD is able to deliver the fluorescent dye into the inside of a cell or not. Alternative assays know for a skilled person in the art may also be suitable to determine the function of a PTD, such as e.g. a luciferase based assay. PTD in combination with fluorescein as cargo are describe in Van den Berg and Dowdy, Curr Opin Biotech, 22, 2011 and in more detail in Vives at el., J Biol Chem, 272, 1997. In the context of mycobacteria infection, the function of a PTD according to the present invention is preferably determined according to the following: cells infected with mycobacteria are contacted with the compositions of first and second fusion proteins of the present invention. The PTD delivers the first and second fusion protein of the composition of the invention inside of the infected cell. The function of the PTD can then be shown by assessing the intracellular survival of the mycobacteria. Preferably this intracellular survival of the mycobacteria is assessed by lysing of the infected cells, such as infected eukaryotic cells, e.g. macrophages, which have been contacted with a composition of first and second fusion protein of the present invention, and e.g. plating of serial dilutions of the lysed cells on agar plates. Colonies of mycobacteria which may have survived the treatment with the compositions of the present invention, and are grown on the agar plate can be enumerated after a distinct incubation period. Accordingly, a person skilled in the art is able to determine the function of the PTD of the first and second fusion protein of the present invention by determining the intracellular survival of mycobacteria. Distinct methods for the determination of the intracellular survival of mycobacteria, such as e.g. plating of serial dilutions on agar plates, or determination of mycobacteria via PCR methods, such as quantitative PCR methods, are known to the person skilled in the art.

According to the present invention, a functionally effective PTD in the first and second fusion protein of the composition of the present invention is given if a lower intracellular survival rate of the mycobacteria in infected samples after contacting with the composition of the present invention can be determined in comparison to the survival rate of untreated infected samples.

In a preferred embodiment of the present invention is the intracellular survival rate of mycobacteria infected samples contacted with the composition of the present invention preferably about 0-10%, 10-20%, 20-30%, 30-40%, 40-50%, 50-60%, 60-70%, 70-80%, 80-90%, or 90-99.9% in comparison to the intracellular survival rate of mycobacteria in untreated infected samples.

The term “deletion” as used herein refers to the removal of 1, 2, 3, 4, 5 or more amino acid residues from the respective starting sequence.

The term “insertion” or “addition” as used herein refers to the insertion or addition of 1, 2, 3, 4, 5 or more amino acid residues to the respective starting sequence.

The term “substitution” as used herein refers to the exchange of an amino acid residue located at a certain position for a different one.

The term “cell wall” as used herein refers to all components that form the outer cell enclosure in particular of a Mycobacterium and thus guarantee their integrity. In particular, the term “cell wall” as used herein refers to in particular to the arabinogalactan layer and the mycolic acid layer of Mycobacteria, but also to membranes or additional layers deposited or attached to the mycolic acid layer, such as capsule-like material, outer protein layer or slimes.

The term “EAD” as used herein refers to the enzymatically active domain of an endolysin. The EAD is responsible for hydrolysing bacterial peptidoglycans. It exhibits at least one enzymatic activity of an endolysin. The EAD can also be composed of more than one enzymatically active module. The term “EAD” is used herein synonymously with the term “catalytic domain”.

As used herein, the term “cationic peptide” refers to a synthetic peptide having positively charged amino acid residues. Preferably a cationic peptide has a pKa-value of 9.0 or greater. Typically, at least four of the amino acid residues of the cationic peptide can be positively charged, for example, lysine or arginine. “Positively charged” refers to the side chains of the amino acid residues which have a net positive charge at about physiological conditions. The term “cationic peptide” as used herein refers also to polycationic peptides.

The term “polycationic peptide” as used herein refers to a synthetically produced peptide composed of mostly positively charged amino acid residues, in particular lysine and/or arginine residues. A peptide is composed of mostly positively charged amino acid residues of at least about 20, 30, 40, 50, 60, 70, 75, 80, 85, 90, 95 or about 100% of the amino acid residues are positively charged amino acid residues, in particular lysine and/or arginine residues. The amino acid residues being not positively charged amino acid residues can be neutrally charged amino acid residues and/or negatively charged amino acid residues and/or hydrophobic amino acid residues. Preferably the amino acid residues being not positively charged amino acid residues are neutrally charged amino acid residues, in particular serine and/or glycine.

The term “antimicrobial peptide” (AMP) as used herein refers to any naturally occurring peptide that has microbicidal and/or microbistatic activity in particular against a Mycobacterium species. Thus, the term “antimicrobial peptide” as used herein relates in particular to any peptide having anti-bacterial, anti-infectious, anti-infective and/or germicidal, microbicidal, or bactericidal properties.

The antimicrobial peptide may be a member of the RNAse A super family, a defensin, cathelicidin, granulysin, histatin, psoriasin, dermicidine or hepcidin. The antimicrobial peptide may be naturally occurring in insects, fish, plants, arachnids, vertebrates or mammals. Preferably the antimicrobial peptide may be naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human.

The term “amphiphatic peptide” as used herein refers to synthetic peptides having both hydrophilic and hydrophobic functional groups. Preferably, the term “amphiphatic peptide” as used herein refers to a peptide having a defined arrangement of hydrophilic and hydrophobic groups e.g. amphipathic peptides may be e.g. alpha helical, having predominantly non polar side chains along one side of the helix and polar residues along the remainder of its surface.

The term “hydrophobic group” as used herein refers to chemical groups such as amino acid side chains which are substantially water insoluble, but soluble in an oil phase, with the solubility in the oil phase being higher than that in water or in an aqueous phase. In water, amino acid residues having a hydrophobic side chain interact with one another to generate a nonaqueous environment. Examples of amino acid residues with hydrophobic side chains are valine, isoleucine, leucine, methionine, phenylalanine, tryptophan, cysteine, alanine, tyrosine, histidine, threonin, serine, proline and glycine residues.

The term “autolysins” refers to enzymes related to endolysins but encoded by bacteria and involved in e.g. cell division. An overview of autolysins is can be found in “Bacterial peptidoglycan (murein) hydrolases. Vollmer W, Joris B, Charlier P, Foster S. FEMS Microbiol Rev. 2008 March; 32(2):259-86”.

The term “bacteriocin” as used herein refers to protein-like, polypeptide-like or peptide-like substances which are able to inhibit the growth of other bacteria. Some bacteriocins are capable of degrading bacterial cell walls like Lysostaphin (degrading Staphylococcus cell walls), Mutanolysin (degrading Streptococcus cell walls) and Enterolysin (degrading Enterococcus cell walls). Preferably said growth inhibition is specifically by means of absorption of said other bacteria to specific receptors of the bacteriocin. A further group of bacteriocins are Nisin-like peptides (Gene encoded antimicrobial peptides, a template for the design of novel anti-mycobacterial drugs. Carroll J, Field D, O'Connor P M, Cotter P D, Coffey A, Hill C, Ross R P, O'Mahony J. Bioeng Bugs. 2010 November-December; 1(6):408-12). In general, bacteriocins are produced by microorganisms. However, the term “bacteriocin” as used herein refers both to an isolated form procuded by a microorganism or to a synthetically produced form, and refers also to variants which substantially retain the activities of their parent bacteriocins, but whose sequences have been altered by insertion or deletion of one or more amino acid residues.

The present invention relates to new antibacterial agents against Mycobacterium species, in particular to a composition having the activity of degrading the cell wall of a Mycobacterium species comprising: (a) a first fusion protein including (i) a first endolysin or a first domain, both having a first enzymatic activity, the enzymatic activity being at least one or more of the following: N-acetyl-b-D-muramidase (lysozyme, lytic transglycosylase), N-acetyl-b-D-glucosaminidase, N-acetylmuramoyl-L-alanine amidase, L-alanoyl-D-glutamate (LD) endopeptidase, c-D-glutamyl-meso-diaminopimelic acid (DL) peptidase, D-Ala-m-DAP (DD) endopeptidase, or m-DAP-m-DAP (LD) endopeptidase, (ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (iii) a protein transduction domain (PTD) being at the N- or C-terminus of the first fusion protein, wherein the PTD is having the characteristic to deliver a cargo from the extracellular to the intracellular space of a cell; and (b) a second fusion protein including (i) a second endolysin or a second domain, both having a second enzymatic activity, the enzymatic activity being at least one or more of the following: lipolytic activity, cutinase, mycolarabinogalactanesterase, or alpha/beta hydrolase; (ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (iii) a protein transduction domain (PTD) being at the N- or C-terminus of the second fusion protein, wherein the PTD is having the characteristic to deliver a cargo from the extracellular to the intracellular space of a cell.

In the context of mycobacteria infection, it is known that mycobacteria may be present inside of cells, such as macrophages. Therefore, there are some difficulties to reach mycobacteria which reside intracelluarly and which are thus not directly accessible. Therefore, according to the present invention the PTD functions to deliver the first and second fusion protein of the present invention inside of a cell, such as preferably eukaryotic cells, e.g. macrophages.

In the context of mycobacteria infection, the function of a PTD according to the present invention is preferably determined according to the following: cells infected with mycobacteria are contacted with the compositions of first and second fusion proteins of the present invention. The PTD delivers the first and second fusion protein of the composition of the invention inside of the infected cell. The function of the PTD can then be shown by assessing the intracellular survival of the mycobacteria. Preferably this intracellular survival of the mycobacteria is assessed by lysing of the infected cells, such as infected eukaryotic cells, e.g. macrophages, which have been contacted with a composition of first and second fusion protein of the present invention, and e.g. plating of serial dilutions of the lysed cells on agar plates. Colonies of mycobacteria which may have survived the treatment with the compositions of the present invention, and are grown on the agar plate can be enumerated after a distinct incubation period. Accordingly, a person skilled in the art is able to determine the function of the PTD of the first and second fusion protein of the present invention by determining the intracellular survival of mycobacteria. Distinct methods for the determination of the intracellular survival of mycobacteria, such as e.g. plating of serial dilutions on agar plates, or determination of mycobacteria via PCR methods, such as quantitative PCR methods, are known to the person skilled in the art.

According to the present invention, a functionally effective PTD in the first and second fusion protein of the composition of the present invention is given if a lower intracellular survival rate of the mycobacteria in infected samples after contacting with the composition of the present invention can be determined in comparison to the survival rate of untreated infected samples.

In a preferred embodiment of the present invention is the intracellular survival rate of mycobacteria infected samples contacted with the composition of the present invention preferably about 0-10%, 10-20%, 20-30%, 30-40%, 40-50%, 50-60%, 60-70%, 70-80%, 80-90%, or 90-99.9% in comparison to the intracellular survival rate of mycobacteria in untreated infected samples.

Accordingly, the present invention relates to a composition comprising two distinct fusion proteins which include distinct enzymatic activities combined with a peptide stretch and a protein transduction domain. This distinct combination of a first and a second fusion protein, wherein the first and the second fusion protein include each a first and a second enzymatic activity, a peptide stretch and the PTD, in a composition according to the present invention is advantageous. The composition according to the present invention provides the distinct advantage that the combination of two fusion proteins exert a strong activity in respect of the degradation of the cell wall of Mycobacteria species. Moreover, due to the presence of the PTD in each of the both fusion proteins, the structure of the outer cell membrane can be more easily overcome with the present invention. This is in particular of importance in the case that the mycobacteria are surviving inside of a host cell, such as in a human macrophage. Therefore, the composition of the present invention is able to provoke a strong anti-mycobacterial activity.

In a preferred embodiment of the composition of the present invention the first endolysin is Lysin A (LysA) or the first enzymatic activity of the first domain is exerted by Lysin A (LysA) or Lysin A like enzymes and the second endolysin is Lysin B (LysB) or the second enzymatic activity of the second domain is exerted by Lysin B (LysB) or Lysin B like enzymes.

Examples for the first and second endolysins or the first and second enzymatic activity of the first and second domain are listed in the following table 1.

TABLE 1

| | | type of | | |
|---|---|---|---|---|
| Mycobacte- | endolysin/ | endolysin | amino acid | nucleic acid |
| riophage | domain | or domain | sequence | sequence |
| TM4 | TM4gp29 | Lysin A | SEQ ID NO: 1 | SEQ ID NO: 2 |
| Bxz2 | Bxz2gp11 | Lysin A | SEQ ID NO: 3 | SEQ ID NO: 4 |
| D29 | D29gp10 | Lysin A | SEQ ID NO: 5 | SEQ ID NO: 6 |
| L5 | L5gp10 | Lysin A | SEQ ID NO: 7 | SEQ ID NO: 8 |
| TM4 | TM4gp30 | Lysin B | SEQ ID NO: 9 | SEQ ID |
| | | | | NO: 10 |
| Bxz2 | Bxz2gp12 | Lysin B | SEQ ID NO: 11 | SEQ ID |
| | | | | NO: 12 |
| D29 | D29gp12 | Lysin B | SEQ ID NO: 13 | SEQ ID |
| | | | | NO: 14 |
| L5 | L5gp12 | Lysin B | SEQ ID NO: 15 | SEQ ID |
| | | | | NO: 16 |

In a preferred embodiment the first and the second enzymatic activity of the first and second fusion protein of the composition is exerted by enzymes derived from mycobacteriophages selected from the group consisting of TM4, D29, L5, and Bxz2.

In a further preferred embodiment the peptide stretch of the first and second fusion protein of the composition of the present invention comprises an antimicrobial peptide (AMP), the AMP being selected from the group consisting of Cathelicidins (hCAP-18/LL37), alpha defensins, beta defensins, hepcidin, NK-2, and Ci-MAM-A24.

Examples for antimicrobial peptides according to the present invention are listed in the following table 2.

TABLE 2

| | | nucleic acid |
|---|---|---|
| Peptid | amino acid sequence | sequence |
| LL-37 | LLGDFFRKSKEKIGKEFKRIVQRIKDFLR | SEQ ID NO: 18 |
| | NLVPRTES | |
| | SEQ ID NO: 17 | |
| alpha- | DCYCRIPACIAGERRYGTCIYQGRLWAF | SEQ ID NO: 20 |
| defensin | CC | |
| | SEQ ID NO: 19 | |
| beta- | NPVSCVRNKGICVPIRCPGSMKQIGTCVG | SEQ ID NO: 22 |
| defensin | RAVKCCRKK | |
| | SEQ ID NO: 21 | |
| Hepcidin | DTHFPICIFCCGCCHRSKCGMCCKT | SEQ ID NO: 24 |
| | SEQ ID NO: 23 | |
| NK-2 | KILRGVCKKIMRTFLRRISKDILTGKK; | SEQ ID NO: 26 |
| | SEQ ID NO: 25 | |
| Ci-MAM- | WRSLGRTLLRLSHALKPLARRSGW | SEQ ID NO: 28 |
| A24 | SEQ ID NO: 27 | |

In a preferred embodiment the PTD of the first and second fusion protein of the composition of the present invention is selected from the group consisting of TAT48-60, TAT47-57, TAT47-55, PTD3, PolyArginine, CADY, PepFect6, RXR, Antennapedia, Kala Syn, M918, MAP, Penetratin, PTD5-Syn, Pvec, Poly Arg 8, TAT 48-60, Transportan, Transportan10, and TAT-9.

Examples for PTDs according to the present invention are listed in the following table 3.

TABLE 3

| PTD | amino acid sequence | SEQ ID NO |
|---|---|---|
| TAT48-60 Ref. 1 | GRKKRRQRRRPPQC | SEQ ID NO: 29 |
| TAT47-57 Ref. 1 | YGRKKRRQRRR | SEQ ID NO: 30 |
| TAT47-55 Ref. 1 | YGRKKRRQR | SEQ ID NO: 31 |
| PTD3 Ref. 1 | YARKARRQARR | SEQ ID NO: 32 |
| PolyArginine | RRRRRRRRR | SEQ ID NO: 33 |
| Ref. 1 | | |
| CADY Ref. 1 | GLWRALWRLLRSLWRLLWRA | SEQ ID NO: 34 |
| PepFect6 Ref. 1 | AGYLLGKINLKALAALAKKIL | SEQ ID NO: 35 |
| RXR Ref. 1 | RXRRXRRXRRXRXB | SEQ ID NO: 36 |
| Antennapedia | RQIKIWFQNRRMKWKK | SEQ ID NO: 37 |
| Ref. 2 | | |
| Kala Syn Ref. 3 | WEAKLAKALAKALAKHL | SEQ ID NO: 38 |
| | AKALAKALKACEA | |
| M918 Ref. 4 | MVTVLFRRLRIRRASGPPRV | SEQ ID NO: 39 |
| | RV | |
| MAP Ref. 5 | KLALKLALKALKAALKLA | SEQ ID NO: 40 |
| Penetratin | RQIKIWFQNRRMKWKK | SEQ ID NO: 41 |
| Ref. 6 | | |
| PTD5-Syn Ref. 7 | RRQRRTSKLMKR | SEQ ID NO: 42 |
| Pvec Ref. 8 | LLIILRRRIRKQAHAHSK | SEQ ID NO: 43 |
| Poly Arg 8 | RRRRRRRR | SEQ ID NO: 44 |
| Ref. 2 | | |
| TAT48-60 Ref. 9 | GRKKRRQRRRPPQ | SEQ ID NO: 45 |
| Transportan | GWTLNSAGYLLGKINLKA | SEQ ID NO: 46 |
| Ref. 10 | LAALAKKIL | |
| Transportan10 | AGYLLGKINLKALAALAKKIL | SEQ ID NO: 47 |
| Ref. 11 | | |
| TAT-9 Ref. 9 | RKKRRQRRR | SEQ ID NO: 48 |

The PTDs are disclosed in the following references:

1. Van den Berg and Dowdy, Curr Opin Biotech, 22, 2011. 2. Kabouridis, P. S. Biological applications of protein transduction technology. Trends Biotechnol. 21, 498-503 (2003). 3. Min, S. H. et al. Gene delivery using a derivative of the protein transduction domain peptide, K-Antp. Biomaterials. 31, 1858-1864 (2010). 4. El-Andaloussi, S., Johansson, H. J., Holm, T., & Langel, U. A novel cell-penetrating peptide, M918, for efficient delivery of proteins and peptide nucleic acids. Mol. Ther. 15, 1820-1826 (2007). 5. Saar, K. et al. Cell-penetrating peptides: a comparative membrane toxicity study. Anal. Biochem. 345, 55-65 (2005). 6. Splith, K. & Neundorf, I. Antimicrobial peptides with cell-penetrating peptide properties and vice versa. Eur. Biophys. J. (2011). 7. Mi, Z. et al. Identification of a synovial fibroblast-specific protein transduction domain for delivery of apoptotic agents to hyperplastic synovium. Mol. Ther. 8, 295-305 (2003). 8. Herbig, M. E. et al. Bilayer interaction and localization of cell penetrating peptides with model membranes: a comparative study of a human calcitonin (hCT)-derived peptide with pVEC and pAntp(43-58). Biochim Biophys. Acta. 1712, 197-211 (2005). 9. Eguchi, A. et al. Protein transduction domain of HIV-1 Tat protein promotes efficient delivery of DNA into mammalian cells. J. Biol. Chem. 276, 26204-26210 (2001). 10. Jones, S. W. et al. Characterisation of cell-penetrating peptide-mediated peptide delivery. Br. J. Pharmacol. 145, 1093-1102 (2005). 11. Fisher, L. et al. Cellular delivery of a double-stranded oligonucleotide NFkappaB decoy by hybridization to complementary PNA linked to a cell-penetrating peptide. Gene Ther. 11, 1264-1272 (2004).

In a further preferred embodiment the composition of the present invention is having activity of degrading the cell wall of a Mycobacterium species which is selected from the group consisting of Mycobacterium tuberculosis, Mycobacterium microti, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium abscessus, Mycobacterium kansasii, Mycobacterium terrae, Mycobacterium nonchromogenicum, Mycobacterium gordonae, and Mycobacterium triviale.

In a preferred embodiment of the composition of the present invention, the domain with the first enzymatic activity of the first fusion protein exhibits an amino acid sequence selected from the group consisting of SEQ ID NO:1, 3, 5, and 7, wherein the domain with the second enzymatic activity of the second fusion protein exhibits an amino acid sequence selected from the group consisting SEQ ID NO:9, 11, 13, and 15, wherein the peptide stretch of the first and second fusion protein exhibits an amino acid sequence selected from the group consisting SEQ ID NO:17, 19, 21, 23, 25, and 27, and wherein the PTD exhibits an amino acid sequence selected from the group consisting SEQ ID NO:29, 30, 31, 32, 33, 34, 35, 36, 37, 38, 39, 40, 41, 42, 43, 44, 45, 46, 47, and 48.

In a particular preferred embodiment of the composition of the present invention, the first fusion protein is exhibiting an amino acid sequence selected from the group consisting SEQ ID NO:49, 51, 53, 55, 57, 59, 61, 77, 79, 81, 89, 81, 93, 95, 97, 99, 113, and 115, and the second fusion protein is exhibiting an amino acid sequence selected from the group consisting SEQ ID NO:63, 65, 67, 69, 71, 73, 75, 83, 85, 87, 101, 103, 105, 107, 109, 111, and 117.

Specific examples of fusion proteins according to the present invention are listed in the following table.

TABLE 4

| | amino acid | nucleic acid |
|---|---|---|
| First fusion protein | sequence | sequence |
| TM4gp29/LL-37/TAT47-57 | SEQ ID NO: 49 | SEQ ID NO: 50 |
| TM4gp29/LL-37/TAT47-57 | SEQ ID NO: 51 | SEQ ID NO: 52 |
| Bzx2gp11/alpha-defensin/PTD3 | SEQ ID NO: 53 | SEQ ID NO: 54 |
| PTD3/alpha-defensin/Bzx2gp11 | SEQ ID NO: 55 | SEQ ID NO: 56 |
| PTD3/alpha- | SEQ ID NO: 57 | SEQ ID NO: 58 |
| defensin/Bzx2gp11/alpha-defensin | | |
| beta-defensin/L5gp10/TAT47-57 | SEQ ID NO: 59 | SEQ ID NO: 60 |
| alpha- | SEQ ID NO: 61 | SEQ ID NO: 62 |
| defensin/Hepcidin/L5gp10/TAT47- | | |
| 57 | | |
| TM4gp29/LL-37/TAT47-57 | SEQ ID NO: 77 | SEQ ID NO: 78 |
| TM4gp29/LL-37/TAT47-57 | SEQ ID NO: 79 | SEQ ID NO: 80 |
| Bzx2gp11/alpha-defensin/PTD3 | SEQ ID NO: 81 | SEQ ID NO: 82 |
| TM4gp29/LL-37/TAT47-57 | SEQ ID NO: 89 | SEQ ID NO: 90 |
| Bzx2gp11/alpha-defensin/PTD3 | SEQ ID NO: 91 | SEQ ID NO: 92 |
| PTD3/alpha-defensin/Bzx2gp11 | SEQ ID NO: 93 | SEQ ID NO: 94 |
| PTD3/alpha- | SEQ ID NO: 95 | SEQ ID NO: 96 |
| defensin/Bzx2gp11/alpha-defensin | | |
| beta-defensin/L5gp10/TAT47-57 | SEQ ID NO: 97 | SEQ ID NO: 98 |
| alpha- | SEQ ID NO: 99 | SEQ ID NO: 100 |
| defensin/Hepcidin/L5gp10/TAT47- | | |
| 57 | | |
| TM4gp29/LL-37/TAT47-57 | SEQ ID NO: 113 | SEQ ID NO: 114 |
| PTD3/alpha-defensin/Bzx2gp11 | SEQ ID NO: 115 | SEQ ID NO: 116 |
| | amino acid | nucleic acid |
| Second fusion protein | sequence | sequence |
| TM4gp30/LL-37/TAT47-57 | SEQ ID NO: 63 | SEQ ID NO: 64 |
| TM4gp30/LL-37/TAT47-57 | SEQ ID NO: 65 | SEQ ID NO: 66 |
| D29gp12/alpha-defensin/PTD3 | SEQ ID NO: 67 | SEQ ID NO: 68 |
| PTD3/alpha-defensin/D29gp12 | SEQ ID NO: 69 | SEQ ID NO: 70 |
| PTD3/alpha- | SEQ ID NO: 71 | SEQ ID NO: 72 |
| defensin/D29gp12/alpha-defensin | | |
| beta-defensin/D29gp12/TAT47-57 | SEQ ID NO: 73 | SEQ ID NO: 74 |
| beta- | SEQ ID NO: 75 | SEQ ID NO: 76 |
| defensin/Hepcidin/L5gp12/TAT47- | | |
| 57 | | |
| TM4gp30/LL-37/TAT47-57 | SEQ ID NO: 83 | SEQ ID NO: 84 |
| TM4gp30/LL-37/TAT47-57 | SEQ ID NO: 85 | SEQ ID NO: 86 |
| D29gp12/alpha-defensin/PTD3 | SEQ ID NO: 87 | SEQ ID NO: 88 |
| TM4gp30/LL-37/TAT47-57 | SEQ ID NO: 101 | SEQ ID NO: 102 |
| D29gp12/alpha-defensin/PTD3 | SEQ ID NO: 103 | SEQ ID NO: 104 |
| PTD3/alpha-defensin/D29gp12 | SEQ ID NO: 105 | SEQ ID NO: 106 |
| PTD3/alpha- | SEQ ID NO: 107 | SEQ ID NO: 108 |
| defensin/D29gp12/alpha-defensin | | |
| beta-defensin/D29gp12/TAT47-57 | SEQ ID NO: 109 | SEQ ID NO: 110 |
| beta- | SEQ ID NO: 111 | SEQ ID NO: 112 |
| defensin/Hepcidin/L5gp12/TAT47- | | |
| 57 | | |
| beta-defensin/D29gp12/TAT47-57 | SEQ ID NO: 117 | SEQ ID NO:118 |

In another preferred embodiment of the present invention the enzymes, such as endolysins, autolysins and bacteriocins of the first and second fusion protein according to the present invention comprise modifications and/or alterations of the amino acid sequences. Such alterations and/or modifications may comprise mutations such as deletions, insertions and additions, substitutions or combinations thereof and/or chemical changes of the amino acid residues, e.g. biotinylation, acetylation, pegylation, chemical changes of the amino-, SH- or carboxyl-groups. Said endolysins, autolysins and bacteriocins of the fusion protein according to the present invention exhibit the lytic activity of the respective wild-type endolysin, autolysin and bacteriocin. However, said activity can be the same, higher or lower as the activity of the respective wild-type endolysin, autolysin and bacteriocin. Said activity can be about 10, 20, 30, 40, 50, 60, 70, 80, 90, 100, 110, 120, 130, 140, 150, 160, 170, 180, 190 or about 200% of the activity of the respective wild-type endolysin, autolysin and bacteriocin or even more. The activity can be measured by assays well known in the art by a person skilled in the art as e.g. the plate lysis assay or the liquid lysis assay which are e.g. described in Briers et al., J. Biochem. Biophys Methods 70: 531-533, (2007), Donovan D M, Lardeo M, Foster-Frey J. FEMS Microbiol Lett. 2006 December; 265(1), Payne K M, Hatfull G F PLoS One, 2012.

The peptide stretch and the PTD of the fusion proteins according to the present invention may be linked to the endolysin or the domain having an enzymatic activity by additional amino acid residues e.g. due to cloning reasons. Preferably, said additional amino acid residues may be not recognized and/or cleaved by proteases. Preferably the peptide stretch and the PTD may be linked to the endolysin or the domain having an enzymatic activity by at least 1, 2, 3, 4, 5, 6, 7, 8, 9 or 10 additional amino acid residues. In a preferred embodiment the peptide stretch is fused to the N- or C-terminus of the endolysin or the domain having an enzymatic activity by the additional amino acid residues glycine, serine, and alanine (Gly-Ser-Ala). Moreover, the PTD is located on the N-terminus or on the C-Terminus of the first fusion protein or of the second fusion protein according to the invention.

The PTD may further comprise additional amino acids on its N- or C-terminus. Preferably the peptide stretch or the PTD comprise the amino acid methionine (Met), or methionine, glycine and serine (Met-Gly-Ser). In another preferred embodiment the first peptide stretch is linked to the N-terminus of the enzyme by the additional amino acid residues, in particular glycine and serine (Gly-Ser) and the second peptide stretch is linked to the N-terminus of the first peptide stretch by the additional amino acid residues, in particular glycine and serine (Gly-Ser). In another preferred embodiment the first peptide stretch is linked to the C-terminus of the enzyme by the additional amino acid residues, in particular glycine and serine (Gly-Ser) and the second peptide stretch is linked to the C-terminus of the first peptide stretch by the additional amino acid residues, in particular glycine and serine (Gly-Ser).

Within the first and second fusion protein according to the present invention the peptide stretch and the PTD are preferably covalently bound to the endolysin or to the domain, both having enzymatic activity. Preferably, the peptide stretch and the PTD consist of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acid residues. Especially preferred are peptide stretches and PTDs comprising about 5 to about 100 amino acid residues, about 5 to about 50 or about 5 to about 30 amino acid residues. More preferred are peptide stretches and PTDs comprising about 6 to about 42 amino acid residues, about 6 to about 39 amino acid residues, about 6 to about 38 amino acid residues, about 6 to about 31 amino acid residues, about 6 to about 25 amino acid residues, about 6 to about 24 amino acid residues, about 6 to about 22 amino acid residues, about 6 to about 21 amino acid residues, about 6 to about 20 amino acid residues, about 6 to about 19 amino acid residues, about 6 to about 16 amino acid residues, about 6 to about 14 amino acid residues, about 6 to about 12 amino acid residues, about 6 to about 10 amino acid residues or about 6 to about 9 amino acid residues.

Preferably, the peptide stretches and the PTDs are no tag such as a His-tag, Strep-tag, Avi-tag, Myc-tag, Gst-tag, JS-tag, cystein-tag, FLAG-tag or other tags known in the art and no thioredoxin or maltose binding proteins (MBP). However, the first and second fusion protein according to the present invention may comprise in addition such tag or tags.

More preferably the peptide stretches and in particular the PTDs have the function to lead the first and second fusion protein of the composition of the present invention through the outer membrane but may have activity or may have no or only low activity when administered without being fused to the endolysin or the domain, both having enzymatic activity. The function to lead the first and second fusion protein through the outer membrane of mycobacteria is caused by the potential of the outer membrane or mycolic acid/arabinogalactan or LPS disrupting or permeabilising or destabilizing activity of said peptide stretches in combination with the PTDs and the endolysins or the domains. Such outer membrane or LPS disrupting or permeabilising or destabilizing activity of the peptide stretches may be determined in a method as follows: The bacteria cells to be treated are cultured in liquid medium or on agar plates. Then the bacteria cell concentration in the liquid medium is determined photometrically at OD600 nm or the colonies on the agar plates are counted, respectively. Now, the bacteria cells in liquid medium or on the plates are treated with a first and second fusion protein according to the invention. After incubation the bacteria cell concentration in the liquid medium is determined photometrically at OD600 nm or the colonies on the agar plates are counted again. If the first and second fusion protein exhibits such outer membrane disrupting or permeabilising or destabilizing activity, the bacteria cells are lysed due to the treatment with the fusion protein and thus, the bacteria cell concentration in the liquid medium or the number of the bacteria colonies on the agar plate is reduced. Thus, the reduction in bacteria cell concentration or in the number of bacteria colonies after treatment with the first and second fusion protein is indicative for an outer membrane or LPS disrupting or permeabilising or destabilizing activity of the first and second fusion protein.

Fusion proteins are constructed by linking at least three nucleic acid sequences using standard cloning techniques as described e.g. by Sambrook et al. 2001, Molecular Cloning: A Laboratory Manual. Such a protein may be produced, e.g., in recombinant DNA expression systems. Such fusion proteins according to the present invention can be obtained by fusing the nucleic acids for endolysin and the respective peptide stretches.

A further subject-matter of the present invention relates to an isolated nucleic acid molecule encoding the first fusion protein of the composition of to the present invention or to an isolated nucleic acid molecule encoding the second fusion protein of the composition of the present invention.

Preferably the isolated nucleic acid molecule encoding the first fusion protein of the composition of to the present invention is selected from the group consisting of SEQ ID NO:50, 52, 54, 56, 58, 60, 62, 78, 80, 82, 90, 92, 94, 96, 98, 100, 114, and 116. Preferably the isolated nucleic acid molecule encoding the second fusion protein of the composition of to the present invention is selected from the group consisting of SEQ ID NO:64, 66, 68, 70, 72, 74, 76, 84, 86, 88, 102, 104, 106, 108, 110, 112, and 118.

The present invention further relates to a vector comprising a nucleic acid molecule according to the present invention. Said vector may provide for the constitutive or inducible expression of said fusion protein according to the present invention.

The invention also relates to a method for obtaining said first and second fusion protein of the composition of the present invention from a micro-organism, such as a genetically modified suitable host cell which expresses said fusion proteins. Said host cell may be a microorganism such as bacteria or yeast or an animal cell as e.g. a mammalian cell, in particular a human cell. In one embodiment of the present invention the host cell is a Pichia pastoris cell. The host may be selected due to mere biotechnological reasons, e.g. yield, solubility, costs, etc. but may be also selected from a medical point of view, e.g. a non-pathological bacteria or yeast, human cells.

Another subject-matter of the present invention relates to a method for genetically transforming a suitable host cell in order to obtain the expression of the first and second fusion protein of the composition according to the invention, wherein the host cell is genetically modified by the introduction of a genetic material encoding said fusion proteins into the host cell and obtain their translation and expression by genetic engineering methods well known by a person skilled in the art.

In a further aspect the present invention relates to a pharmaceutical composition, comprising a composition comprising a first and a second fusion protein according to the present invention and/or a host transformed with a nucleic acid molecule or a vector comprising a nucleotide sequence encoding a first fusion protein and a nucleic acid molecule or a vector comprising a nucleotide sequence encoding a second fusion protein of the composition according to the present invention.

The present invention also relates to a composition according to the present invention and/or a host transformed with a nucleic acid comprising a nucleotide sequence encoding a first fusion protein or a nucleic acid molecule encoding a second fusion protein of the composition according to the present invention for use as a medicament.

In a further subject-matter the present invention relates to the use of a composition comprising the first and the second fusion protein according to the present invention and/or a host transformed with a vector comprising the nucleic acid molecule comprising a nucleotide sequence encoding a modified first fusion protein or a nucleotide sequence encoding a modified second fusion protein of the composition according to the present invention in the manufacture of a medicament for the treatment and/or prevention of a disorder, disease or condition associated with a Mycobacterium species.

The present invention relates further to a composition comprising of the first and second fusion protein according to the present invention for use as a medicament, a disinfectant, a feed additive, or food additive.

Preferably, a composition comprising the first and second fusion protein according to the present invention is foreseen for use as a medicament, a disinfectant, feed additive, or food additive in the treatment or prevention of diseases caused by an infection with a Mycobacterium species, such as tuberculosis, leprosy, pulmonary disease, lymphadenitis or skin disease.

In a further preferred embodiment a composition comprising a first and a second fusion protein of the present invention is foreseen for use as a medicament, a disinfectant, feed additive, or food additive in the treatment or prevention of a Mycobacterial contamination of foodstuff, of food processing equipment, of food processing plants, of surfaces coming into contact with foodstuff, of medical devices, or of surfaces in hospitals and surgeries.

In another preferred embodiment a composition comprising a first and a second fusion protein of the present invention is foreseen for use as a medicament, a disinfectant or food additive, wherein the composition is used in combination with an additional therapeutic agent.

Additional therapeutic agents may be medicaments and pharmaceutical active substances e.g. antibiotics, such as streptomycin, tetracycline, cephalothin, gentamicin, cefotaxime, cephalosporin, ceftazidime, imipenem, isoniazid, ethambutol, pyrazinamide, penicillin-streptomycin, clofazimine, capreomycin, dapsone, D-cycloserine, ethionamide, rifampin, rifabutin, or rifapentine.

In a further subject-matter of the present invention relates to a method of treating a disorder, disease or condition in a subject in need of treatment and/or prevention, which method comprises administering to said subject an effective amount of a composition comprising a first and a second fusion protein according to the present invention and/or an effective amount of a host transformed with nucleic acids comprising a nucleotide sequences encoding a first and a second fusion protein according to the present invention or a medicament or a pharmaceutical composition comprising the composition of the present invention. The subject may be a human or an animal.

The dosage and route of administration used in a method of treatment or prophylaxis according to the present invention depends on the specific disease/site of infection to be treated. The route of administration may be for example oral, topical, nasopharyngeal, parenteral, intravenous, rectal or any other route of administration.

For application of a composition according to the present invention and/or an effective amount of a host transformed with a nucleic acid comprising a nucleotide sequences encoding a first and second fusion protein according to the present invention or a medicament or a pharmaceutical composition comprising a composition comprising a first and second fusion protein according to the present invention to a site of infection or site endangered to be infected a formulation may be used that protects the active compounds from environmental influences such as proteases, oxidation, immune response etc., until it reaches the site of infection. Therefore, the formulation may be capsule, dragee, pill, powder, suppository, emulsion, suspension, gel, lotion, cream, salve, injectable solution, syrup, spray, inhalant or any other medical reasonable galenic formulation. Preferably, the galenic formulation may comprise suitable carriers, stabilizers, flavourings, buffers or other suitable reagents. For example, for topical application the formulation may be a lotion, cream, gel, salve or plaster, for nasopharyngeal application the formulation may be saline solution to be applied via a spray to the nose. For oral administration in case of the treatment and/or prevention of a specific infection site e.g. in the intestine, it can be necessary to protect a fusion protein according to the present invention from the harsh digestive environment of the gastrointestinal tract until the site of infection is reached. Thus, bacteria as carrier, which survive the initial steps of digestion in the stomach and which secret later on a first and second fusion protein according to the composition of the present invention into the intestinal environment can be used.

In another specific embodiment of the present invention the disorder, disease or condition is caused by a Mycobacterium species, in particular Mycobacterium tuberculosis, Mycobacterium micron, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium abscessus, Mycobacterium kansasii, Mycobacterium terrae, Mycobacterium nonchromogenicum, Mycobacterium gordonae, and Mycobacterium triviale, in particular tuberculosis.

Preferably, a composition comprising a first and second fusion protein according to the present invention is used for medical treatment, if the infection to be treated or prevented is caused by multiresistant bacterial strains, in particular by strains resistant against one or more of the following antibiotics: streptomycin, tetracycline, cephalothin, gentamicin, cefotaxime, cephalosporin, ceftazidime, imipenem, isoniazid, ethambutol or pyrazinamide. Furthermore, a composition according to the present invention can be used in methods of treatment by administering it in combination with conventional antibacterial agents, such as antibiotics, lantibiotics, bacteriocins or endolysins, etc.

The present invention also relates to a pharmaceutical pack comprising one or more compartments, wherein at least one compartment comprises a composition comprising a first and second fusion protein according to the present invention and/or one or more hosts transformed with a nucleic acid comprising a nucleotide sequences encoding a first and second fusion protein according to the present invention or a medicament or a pharmaceutical composition comprising a composition comprising a first and second fusion protein according to the present invention.

In another subject-matter of the present invention relates to a process of preparation of a pharmaceutical composition, said process comprising admixing one or more fusion protein according to the present invention and/or one or more hosts transformed with a nucleic acid comprising a nucleotide sequence encoding a first and second fusion protein according to the composition of the present invention with a pharmaceutically acceptable diluent, excipient or carrier.

In a further subject-matter of the present invention relates to the use of the composition comprising a first and second fusion protein according to the present invention for the treatment, removal, reduction or prevention of mycobacterial contamination of foodstuff, of food processing equipment, of food processing plants, of surfaces coming into contact with foodstuff such as shelves and food deposit areas and in all other situations, where pathogenic, facultative pathogenic or other undesirable bacteria can potentially infest food material, of medical devices and of all kind of surfaces in hospitals and surgeries.

In a subject-matter of the present invention relates to the use of the composition comprising a first and second fusion protein according to the present invention as a food and feed additive.

Further scope of applicability of the present invention will become apparent from the detailed description given hereinafter, however, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

The following examples explain the present invention but are not considered to be limiting. Unless indicated differently, molecular biological standard methods were used, as e.g., described by Sambrock et al., 1989, Molecular Cloning: A Laboratory Manual, 2nd edition, Cold Spring Harbor Laboratory Press, Cold Spring Harbor, N.Y.

### EXAMPLE 1

### Cloning, Expression and Purification of the Respective Proteins Modified with Various Peptide Stretches and Protein Transduction Domains at the N-Terminus or the C-Terminus

### Proteins

TM4gp29 according to SEQ ID NO:1 is a Lys A-type endolysin originating from Mycobacteria phage TM4. The endolysin TM4gp29 is encoded by the nucleic acid molecule according to SEQ ID NO:2. The nucleic acid molecule according to SEQ ID NO:2 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

Bxz2gp11 according to SEQ ID NO:3 is a Lys A-type endolysin originating from Mycobacteria phage Bxz2. The endolysin Bxz2gp11 is encoded by the nucleic acid molecule according to SEQ ID NO:4. The nucleic acid molecule according to SEQ ID NO:4 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

D29gp10 according to SEQ ID NO:5 is a Lys A-type endolysin originating from Mycobacteria phage D29. The endolysin D29gp10 is encoded by the nucleic acid molecule according to SEQ ID NO:6. The nucleic acid molecule according to SEQ ID NO:6 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

L5gp10 according to SEQ ID NO:7 is a Lys A-type endolysin originating from Mycobacteria phage L5. The endolysin L5gp10 is encoded by the nucleic acid molecule according to SEQ ID NO:8. The nucleic acid molecule according to SEQ ID NO: 8 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

TM4gp30 according to SEQ ID NO: 9 is a Lys B-type endolysin originating from Mycobacteria phage TM4. The endolysin TM4gp30 is encoded by the nucleic acid molecule according to SEQ ID NO: 10. The nucleic acid molecule according to SEQ ID NO: 10 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

Bxz2gp12 according to SEQ ID NO: 11 is a Lys B-type endolysin originating from Mycobacteria phage Bxz2. The endolysin Bxz2gp12 is encoded by the nucleic acid molecule according to SEQ ID NO: 12. The nucleic acid molecule according to SEQ ID NO: 12 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

D29gp12 according to SEQ ID NO: 13 is a Lys B-type endolysin originating from Mycobacteria phage D29. The endolysin D29gp12 is encoded by the nucleic acid molecule according to SEQ ID NO:14. The nucleic acid molecule according to SEQ ID NO: 14 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

L5gp12 according to SEQ ID NO: 15 is a Lys B-type endolysin originating from Mycobacteria phage L5. The endolysin L5gp12 is encoded by the nucleic acid molecule according to SEQ ID NO: 16. The nucleic acid molecule according to SEQ ID NO: 16 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

The following peptide stretches in table 5 were used for production of fusion proteins with the endolysins above:

TABLE 5

| Peptide stretch | amino acid sequence | nucleic acid sequence |
|---|---|---|
| LL-37 | SEQ ID NO: 17 | SEQ ID NO: 18 |
| alpha-defensin | SEQ ID NO: 19 | SEQ ID NO: 20 |
| beta-defensin | SEQ ID NO: 21 | SEQ ID NO: 22 |
| Hepcidin | SEQ ID NO: 23 | SEQ ID NO: 24 |

The following protein transduction domains in table 6 were used for production of fusion proteins with the endolysins above:

TABLE 6

| PTD | amino acid sequence | SEQ ID NO |
|---|---|---|
| TAT47-57 | YGRKKRRQRRR | SEQ ID NO: 30 |
| PTD3 | YARKARRQARR | SEQ ID NO: 32 |

The nucleic acid molecules encoding the respective peptide stretches and protein transduction domains were synthetically produced with a Nde I (5′-CAT ATG-3′) restriction site at the 5′-end of the nucleic acid molecule and a BamH I (5′-GGA TCC-3′) restriction site at the 3′-end of the nucleic acid molecule.

Fusion proteins are constructed by linking at least two nucleic acid sequences using standard cloning techniques as described e.g. by Sambrook et al. 2001, Molecular Cloning: A Laboratory Manual or by sequence extension by PCR with designed primers. Accordingly the nucleic acid molecules encoding the peptide stretches were cleaved in a restriction digestion with the respective restriction enzymes, whereas NdeI, necessary for the pert21b vector system, or NcoI, necessary for the pet32b vector system, was at the 5′ end and XhoI was at the 3′-end of the nucleic acid molecules. If necessary the restriction sites of the 5′-end where changed by PCR reactions using primers designed for the restriction site exchange. Furthermore the lysin sequence was extended by a 6×His-tag which was introduced, by PCR with designed primers, between the 3′ end of the lysin sequence and the lysin 3′-end restriction site. Subsequently the cleaved nucleic acids encoding the proteins of interest were ligated into a modified pet21b expression vector, whereas two STOP-codons where introduced 5′ of the vector-encoded 6×His-tag (unmodified vector obtainable from Novagen, Darmstadt, Germany), or into a modified pet32b expression vector, whereas the sequence encoding the S-tag and the central 6×His-tag was deleted (unmodified vector obtainable from Novagen, Darmstadt, Germany). The used expression vector was also cleaved in a digestion with the respective restriction enzymes, so NdeI and XhoI or NcoI and XhoI respectively, before.

Alternatively, the nucleic acid molecules encoding the peptides stretches were cleaved in a restriction digestion with the respective restriction enzymes Nde I and BamH I and in case of the nucleic acid molecule encoding the peptide stretch and PTD for ligation with the proteins the digestion was performed with the restriction enzymes Nco I and BamH I. Subsequently the cleaved nucleic acids encoding the peptide stretches were ligated into the pET21 b expression vector (Novagen, Darmstadt, Germany), which was also cleaved in a digestion with the respective restriction enzymes Nde I and BamH I before. The cleaved nucleic acid molecule encoding the peptide stretch and PTD for ligation with toxic proteins was ligated into a modified pET32 b expression vector (unmodified vector obtainable from Novagen, Darmstadt, Germany), which was also cleaved in a digestion with the respective restriction enzymes Nco I and BamH I before. The modification of the pET32b expression vector refers to the deletion of the sequence encoding an S-tag and the central His-tag.

Afterwards, the nucleic acid molecules encoding the proteins were cleaved in a digestion with the restriction enzyme BamH I and Xho I, so that the proteins could be ligated into the pET21b expression vector (Novagen, Darmstadt, Germany) and the modified pET32 b expression vector, respectively, which were also cleaved in a digest with the respective restriction enzymes BamH I and Xho I before.

In the case of the peptide stretch, which was introduced by PCR to the C-terminus of the proteins, the resulting fusion protein has a His-tag on the N-terminus, wherein the His-tag is linked to the N-terminus by a linker. For the cloning of the respective nucleic acid molecules the pET32 b expression vector (Novagen, Darmstadt, Germany) was used.

Thus, the nucleic acid molecule encoding the peptide stretch is ligated into the respective vector at the 5′-end of the nucleic acid molecule encoding the respective enzyme. Moreover, the nucleic acid molecule encoding the respective enzyme is ligated into the respective plasmid, so that a nucleic acid molecule encoding a His-tag consisting of six histidine residues is associated at the 3′-end of the nucleic acid molecule encoding the endolysin.

As some fusion proteins may either be toxic upon expression in bacteria, or not homogenous due to protein degradation, the strategy might be to express these fusion proteins fused or linked to other additional proteins. Example for these other additional protein is thioredoxin, which was shown to mediate expression of toxic antimicrobial peptides in E. coli (TrxA mediating fusion expression of antimicrobial peptide CM4 from multiple joined genes in Escherichia coli. Zhou L, Zhao Z, Li B, Cai Y, Zhang S. Protein Expr Purif. 2009 April; 64(2):225-230). In the case of the fusion proteins of the present invention, the peptide was ligated into the modified pET32 b expression vector, so that an additional thioredoxin is associated at the 5′-end of the peptide. The thioredoxin could be removed from the expressed fusion protein by the use of enterokinase, therefore between the nucleic acid molecule encoding the peptide and the nucleic acid molecule encoding the thioredoxin an enterokinase restriction site has been introduced.

The sequence of the fusion proteins of the present invention was controlled via DNA-sequencing and correct clones were transformed into E. coli BL21(DE3) or E. coli BL21(DE3) pLysS (Novagen, Darmstadt, Germany) for protein expression.

Recombinant expression of the fusion proteins according to SEQ ID NO: 49, 51, 53, 55, 57, 59, 61, 63, 65, 67, 69, 71, 73, and 75 as well as the further fusion proteins of the present invention is performed in E. coli BL21 (DE3) cells or E. coli BL21 pLysS (DE3) cells (Novagen, Darmstadt, Germany). The cells were growing until an optical density of OD 600 nm of 0.4 to 0.6 was reached. Then the expression of the fusion protein was induced with 1 mM IPTG (isopropylthiogalactoside) and the expression was performed at 37° C. for a period of 4 hours, alternatively an overnight expression at 16° C. was performed.

E. coli BL21 cells were harvested by centrifugation for 20 min at 6000 g and disrupted via sonication on ice. Soluble and insoluble fraction of the E. coli crude extract were separated by centrifugation (Sorvall, SS34, 30 min, 15 000 rpm). All proteins were purified by Ni²⁺ affinity chromatography (Aekta FPLC, GE Healthcare) using the 6×His-tag, encoded by the protein sequence.

Toxic proteins were expressed using a modified pET32b vector (S-tag and central His-tag deleted), which fuses thioredoxin on the N-terminus of the proteins of interest. The vector also contains an enterokinase cleavage site right before the protein of interest. This site allows the proteolytic cleavage between thioredoxin and the protein of interest, which can purified via the remaining C-terminal His-tag. Expressed fusion proteins were not toxic to the host resulting in high yields of produced protein. For antimicrobial function of the fusion protein it was necessary to remove the thioredoxin by proteolytic cleavage. Therefore the fusion protein was cleaved with 8-10 units/mg recombinant enterokinase (Novagen, Darmstadt, Germany) to remove the thioredoxin following the protocol provided by the manufacturer, whereas a NaCl concentration of 400 mM was chosen to prevent the aggregation of the protein of interest. After enterokinase cleavage the fusion protein was purified via His-tag purification as described below.

The Ni²⁺ affinity chromatography is performed in 4 subsequent steps, all at room temperature:
1. Equilibration of the Histrap FF 5 ml column (GE Healthcare) with up to 10 column volumes of Washing Buffer (500 mM to 1 M NaCl, 5% Glycerole and 40 mM Hepes on pH 7.4 or 50 mM Tris-HCl on pH 8.2) at a flow rate of 5 ml/min
2. Loading of the total lysate (with wanted fusion protein) on the Histrap FF 5 ml column at a flow rate of 5 ml/min.
   Washing of the column with Washing Buffer until the UV 280 nm signal drops below 10 mAU or 20 column volumes followed by a washing step with 4% Elution buffer (500 mM imidazole, 0.5 M NaCl, 5% glycerol and 40 mM Hepes on pH 7.4 or 50 mM Tris-HCl on pH 8.2) at a flow rate of 5 ml/min until the UV 280 nm signal drops below 5 mAU or 20 column volumes.
3. Elution of bounded fusion proteins from the column with a linear gradient of 15 column volumes of Elution Buffer (500 mM imidazole, 0.5 M NaCl, 5% glycerol and 40 mM Hepes on pH 7.4 and 50 mM Tris-HCl on pH 8.2) to 100% at a flow rate of 5 ml/min

Purified stock solutions of fusion proteins in Elution Buffer (40 mM Hepes pH 7.4 or 50 mM Tris-HCl pH 8.2; 0.5 M NaCl; 500 mM imidazole; 5% glycerol) were at least 60-90% pure as determined visually on SDS-PAGE gels (data not shown).

Alternatively, the Ni²⁺ affinity chromatography is performed in 4 subsequent steps, all at room temperature:
1. Equilibration of the Histrap FF 5 ml column (GE Healthcare) with up to 10 column volumes of Washing Buffer (20 mM imidazole, 1 M NaCl and 20 mM Hepes on pH 7.4) at a flow rate of 3-5 ml/min
2. Loading of the total lysate (with wanted fusion protein) on the Histrap FF 5 ml column at a flow rate of 3-5 ml/min.
3. Washing of the column with up to 10 column volumes of Washing Buffer to remove unbound sample followed by a second washing step with 10% Elution buffer (500 mM imidazole, 0.5 M NaCl and 20 mM Hepes on pH 7.4) at a flow rate of 3-5 ml/min
4. Elution of bounded fusion proteins from the column with a linear gradient of 4 column volumes of Elution Buffer (500 mM imidazole, 0.5 M NaCl and 20 mM Hepes on pH 7.4) to 100% at a flow rate of 3-5 ml/min

Purified stock solutions of fusion proteins in Elution Buffer (20 mM Hepes pH 7.4; 0.5 M NaCl; 500 mM imidazole) were at least 90% pure as determined visually on SDS-PAGE gels (data not shown).

Lysin A like activity was controlled in a Chloroform assay. Escherichia coli BL21 transformed with the respective Lysin A variant were grown at 37° C. in LB broth supplemented with 100 mg/mL ampicillin to an OD600 nm of 0.5 and then induced with a final concentration of 1 mM IPTG. One hour after induction, 2% chloroform was added to the cell suspension and OD600 nm was monitored. Chloroform permeabilizes the inner membrane, thus replacing the holin function, and allows the putative lysin to reach its target in the peptidoglycan layer. The reduction in OD600 nm after addition of chloroform to 10 mL of induced clones was recorded.

Alternatively, the Micrococcus lysodeikticus Turbidity Reduction Assay has been used as Lysin A activity test. This assay has been adapted from Molecular Microbiology (2009) 71(6), 1509-1522: Structural basis for autoinhibition and activation of Auto, a virulence-associated peptidoglycan hydrolase of Listeria monocytogenes—Maike Bublitz, Lilia Polle, Christin Holland, Dirk W. Heinz, Manfred Nimtz and Wolf-Dieter Schubert. Accordingly, lyophilised cells of Micrococcus Lysodeikticus (Micrococcus lysodeikticus ATCC No. 4698/Sigma-Aldrich/USA/St. Louis) were resuspended in Reaction Buffer (50 mM Hepes, 10 mM MgCl₂, pH 7.4) and diluted to an OD450 nm of 0.5-0.7. Subsequently 400 μl of the cell solution were mixed with 100 μl protein solution, containing 50 μg of the fusion protein of the invention, or 100 μl Protein Storage Buffer (50 mM Tris, 500 mM NaCl 500 mM Imidazole 5% glycerol, pH 8.2). The samples were incubated at 20° C. for 1 h and the decrease of the OD450 nm was measured during the incubation time, whereas the OD450 nm of the samples was determined every 14 seconds. The turbidity reduction graphs were used to determine the ΔOD450 nm with the formula: ΔOD450 nm=OD450 nm_{0sec}−OD450 nm_{3600sec}. Activity has been observed by a steeper turbidity reduction graph, and a resulting increase in the ΔOD450 nm. For the LysinA proteins TM4gp29, Bxz2gp11 and D29gp10 and the fusion proteins based thereon, the turbidity reduction assay revealed good activity.

Lysin B like activity was controlled by enzymatic assays for lipolytic activity like from those described by Payne et al. 2009. Briefly one milliliter of p-nitrophenyl substrates (50 μM) (Sigma) was incubated with 100 μl of the lysine B variants, containing roughly 1 μg dissolved in storage buffer (50 mM Tris-HCl pH 8.2, 500 mM NaCl, 500 mM imidazole, 5% glycerol), or 10 μg of purified native lysine B, dissolved in 100 μl storage buffer, (derived from pET21 or pET32 containing cells) in buffer (20 mM Tris-HCl pH 8.0, 100 mM NaCl, 0.1% Triton X-100) at room temperature for 30 mM Release of p-nitrophenol was determined by measuring absorbance at 420 nm (A420). For the LysinB proteins TM4gp30, Bxz2gp12, L5gp12 and D29gp12 and the fusion proteins based thereon, the lipolytic activity assay revealed good activity.

An overview of distinct parameters of first and second fusion proteins of the compositions of the present invention is given in FIG. 1.

### EXAMPLE 2

### Antimicrobial Activity of Compositions of Fusion Proteins According to the Invention Modified with Various Peptide Stretches and PTDs on the N-Terminus or the C-Terminus Determined with Spot Test on Agar Plates

Liquid overnight cultures of mycobacteria, namely Mycobacterium smegmatis (DSM 43277 termed LiCC 5462, DSM 43468 termed LiCC 5463 and DSM 43756 termed LiCC S464), were grown at 37° C. in Middlebrook 7H9 pH 6.6 medium (0.5 g ammonium sulfate, 0.5 g L-glutamic acid, 0.1 g sodium citrate, 1 mg pyridoxine, 0.5 mg biotin, 2.5 g disodium phosphate, 1 g monopotassium phosphate, 0.04 g ferric ammonium citrate, 0.05 g magnesium sulfate, 0.5 mg calcium chloride, 1 mg zinc sulfate, 1 mg copper sulfate, 4 ml 50% (v/v) glycerol, pH 6.6. After autoclavation at 121° C. for 20 minutes 2.5 ml 20% (v/v) Tween80 were added) at 200 rpm in baffled flasks. The clumps were dispersed, as good as possible, by vortexing. A volume of 100 μl was added to 3 ml Middlebrook 7H9 pH 6.6 top agar and poured onto Middlebrook 7H9 agar plates five-microlitre volumes of the fusion proteins of the present invention were pipetted onto the top agar, whereas for the mixtures a total volume of ten microliters was used (5 μl each protein), and the spots were allowed to dry completely. The plates were incubated for 2 days at 37° C.

Additionally 200 μl liquid overnight culture of mycobacteria were plated on Middlebrook 7H9 agar plates and five-microlitre volumes of the fusion proteins of the present invention were pipetted onto the top agar, whereas for the mixtures a total volume of ten microliters was used (5 μl each protein), and the spots were allowed to dry completely. The plates were incubated for 2 days at 37° C.

The results are shown in tale 7 and 8:

TABLE 7

| | | Composition | |
|---|---|---|---|
| | | comprising the first | |
| | Second fusion | and second fusion | |
| First fusion protein | protein | protein | control |
| SEQ ID NO 49: − | SEQ ID NO 63: − | SEQ ID NO 49 + SEQ | − |
| | | ID NO 63: ++ | |
| SEQID NO 51: − | SEQ ID NO 67: − | SEQ ID NO 51 + SEQ | − |
| | | ID NO 67: ++ | |
| SEQ ID NO 53: − | SEQ ID NO 67: − | SEQ ID NO 53 + SEQ | − |
| | | ID NO 67: + | |
| SEQ ID NO 55: − | SEQ ID NO: 71: − | SEQ ID NO 55 + SEQ | − |
| | | ID NO 73: + | |
| SEQ ID NO 59: − | SEQ ID NO 75: − | SEQ ID NO 59 + SEQ | − |
| | | ID NO 75: ++ | |
| SEQ ID NO 61: − | SEQ ID NO 73: − | SEQ ID NO 61 + SEQ | − |
| | | ID NO 73: + | |
| SEQ ID NO 61: − | SEQ ID NO 69: − | SEQ ID NO 61 + SEQ | − |
| | | ID NO 69: ++ | |

Abbreviations: − no activity; +: 1 log; ++: 2-3 log; +++: 4 log.

The results as shown in table 7 above provide evidence that the first fusion protein and the second fusion protein alone do not exert an anti-mycobacterial activity. In contrast to this, the combination of the first and the second fusion protein in the composition according to the present invention provides a good, in some examples very good activity against mycobacteria.

TABLE 8

| | | Concentration | | | | | | |
|---|---|---|---|---|---|---|---|---|
| | | [5 μl | M. | | M. | | M. | |
| | | or 5 + 5 μl | smegmatis | | smegmatis | | smegmatis | |
| | | addition | S462 | | S463 | | S464 | |
| | Amount | respect.] | | Top | | Top | | Top |
| | [μg] | [mg/ml] | plated | Agar | plated | Agar | plated | Agar |
| Elution buffer B pH 8.2 | 0 | 0 | − | − | − | − | − | − |
| Lysozyme (1 mg/ml) | 5 | 1 | − | − | v | + | − | − |
| Lipase (1 mg/ml) | 5 | 1 | − | − | − | − | − | − |
| Lysozyme + Lipase | 5/5 | 1/1 | 0.9+ | 0.8+ | 1+ | 1+ | 1+ | 0.7+ |
| (1 mg/ml) | | | | | | | | |
| TM4gp29 | 1.06 | 0.212 | v | − | − | − | − | − |
| Bxz2gp11 | 1.41 | 0.282 | − | − | − | − | − | − |
| D29gp10 | 3.695 | 0.739 | − | − | − | − | − | − |
| L5gp12 | 0.765 | 0.153 | v | v | v | v | − | − |
| Pat2 E2 | 0.78 | 0.156 | − | − | − | − | − | − |
| Pat8 | 0.75 | 0.15 | − | + | − | v | − | − |
| Pat3 | 1.165 | 0.233 | − | − | − | − | − | − |
| Pat3 + Pat8 | 1.165/0.75 | 0.233/0.15 | ++ | ++ | +++ | + | + | + |
| Pat2 + Pat8 | 0.78/0.75 | 0.156/0.15 | ++ | + | ++ | + | nd | nd |
| Pat2 + Pat11 | 0.875/0.46 | 0.175/0.092 | ++ | +++ | ++ | + | ++ | ++ |
| Pat11 + Pat8 | 0.46/0.75 | 0.092/0.15 | ++ | +++ | ++ | + | ++ | ++ |
| Pat11 + Pat3 | 0.46/1.165 | 0.092/0.233 | ++ | +++ | ++ | + | nd | ++ |

| | |
|---|---|
| Estimation | appearance |
| Spot not clearly | v |
| detectable | |
| Spot with equal size as | + |
| in control (lysozyme + | |
| lipase) | |
| Spot with greater size | ++ |
| than in control | |
| (lysozyme + lipase) | |
| Spot with even greater | +++ |
| size than in control | |
| (lysozyme + lipase) | |
| No spot | − |
| Not determined | nd |

Diameter in control lysozyme + lipase is given in cm

The results as shown in table 8 above provide evidence that the first and second endolysin of the endolysin type Lysin A and Lysin B alone do not exert an anti-mycobacterial activity, since no spots can be detected in the samples TM4gp29, Bxz2gp11, and D29gp10 as well as L5gp12.

No anti-mycobacterial activity can be seen with one fusion protein alone, as seen in the samples with Bxz2gp11-alpha defensin-PTD3 (Pat2 in table 8), D29gp12-alpha defensin-PTD3 (Pat8 in table 8), and PTD3-alpha defensin-Bxz2gp11 (Pat3 in table 8). This provides evidence that only one fusion protein with Lysin A or Lysin B type of endolysin is not sufficient to achieve lysis of the mycobacteria.

In contrast to this, the combination of the first and the second fusion proteins in the compositions according to the present invention provides a good, in some examples very good activity against mycobacteria. This is in particular shown for the following compositions of first and second fusion proteins: PTD3-alpha defensin-Bxz2gp11 (Pat3 in table 8) and D29gp12-alpha defensin-PTD3 (Pat8 in table 8); Bxz2gp11-alpha defensin-PTD3 (Pat2 in table 8) and D29gp12-alpha defensin-PTD3 (Pat8 in table 8); Bxz2gp11-alpha defensin-PTD3 (Pat2 in table 8) and beta defensin-D29gp12-TAT47-57 (Pat11 in table 8); beta defensin-D29gp12-TAT47-57 (Pat11 in table 8) and D29gp12-alpha defensin-PTD3 (Pat8 in table 8); and beta defensin-D29gp12-TAT47-57 (Pat11 in table 8) and PTD3-alpha defensin-Bxz2gp11 (Pat3 in table 8).

The results of the spot test are also illustrated in FIG. 2. FIG. 2A shows the results of the spot test on bacterial lawn, and FIG. 2b shows the results of the spot test on top agar. The results are also summarized in the following table:

TABLE 9

| Number of the well | Construct | Lysis |
|---|---|---|
| 19 | Pat2 + Pat3 | − |
| 20 | Pat2 + Pat8 | ++ |
| 21 | Pat2 | − |
| 22 | Pat11 | − |
| 23 | Pat2 + Pat11 | ++ |
| 24 | Pat11 + Pat8 | ++ |
| 25 | Pat11 + Pat3 | ++ |
| 26 | Pat2 | − |
| 27 | Pat11 | − |

According to the results as described above, no lysis has been detected on mycobacterial lawns in agar plates only treated with lysin B like fusion proteins (well 19), or in agar plates treated with only one fusion protein (wells 21, 22, 26, and 27).

The above-described results provide evidence that the compositions of the present invention comprising a first and second fusion protein are able to specifically recognize and degrade mycobacteria.

### EXAMPLE 3

### Antimicrobial Activity of Compositions of Fusion Proteins According to the Invention Modified with Various Peptide Stretches and PTDs on the N-Terminus or the C-Terminus Determined with Light Microscopy and Electron Microscopy

### Light Microscopy

Mycobacterial cells from the strains LiCC 5463 and LiCC 5464 were pelleted, washed with reaction puffer (50 mM Hepes, 100 mM NaCl, 10 mM MgCl₂, pH 7.4) and resuspended in reaction buffer to a cell number of roughly 1×10⁷ cells/ml. The cell suspension was mixed in a 9 (cell solution):1 (protein solution) ratio (90 μl cell solution mixed with 10 μl protein solution) with the Lysin-Solution containing a mixture of Construct 2 and Construct 11, namely Bxz2gp11/Alpha-Defensin/PTD3 and Beta-Defensin/D29gp12/TAT47-57 (both with a concentration of 0.32 mg/ml). The samples were incubated at 24° C. for one day and 15 μl samples were taken after 1 h, 2 h, 3 h, 4 h, 5 h and 15 h incubation (overnight) for microscopic analysis. After 2 h increased cell aggregation was observed and after 4 h the morphology of the cells started to change from a normal rod structure to constricted rod structures (data not shown). After 15 h of incubation mostly clusters of cells were observed and less single cells, whereas the single cells seemed hyaline-like.

After 5 h a 20 μl sample was platted on Middlebrook 7H9 agar plates and incubated at 37° C. The control-plate, containing LiCC 5462, showed a lawn after 2 days, whereas the plates with the treated cells showed a lawn after 3 days of incubation, thereby showing a decreased number of living cells or decreased fitness of the living cells in the treated samples (data not shown).

### Electron Microscopy (EM)

Mycobacterial cells from the strain LiCC 5463 were pelleted, washed with reaction puffer (50 mM Hepes, 100 mM NaCl, 10 mM MgCl2, pH 7.4) and resuspended in reaction buffer to a cell number of 5×10⁸ cells/ml. The cell suspension was mixed in a 9 (cell solution):1 (protein solution) ratio (900 μl cell solution mixed with 100 μl sample solution) with a composition of the present invention. As one example a composition of the present invention is used with the fusion proteins of construct 2, namely Bxz2gp11/Alpha-Defensin/PTD3, with a concentration of 0.25 mg/ml, and construct 11, namely Beta-Defensin/D29gp12/TAT47-57, with a concentration of 0.22 mg/ml, a Lysin-mixture-solution of construct 2 and construct 11 or the control solution (50 mM Tris 500 mM NaCl 500 mM Imidazol 5% glycerol pH 8.2). The four samples, construct 1 together with construct 11 (nomenclature see FIG. 1), control with buffer only, construct 2, construct 11, were incubated at 24° C. for 5 h and 15 h and 15 μl samples were taken after 1 h, 2 h, 3 h, 4 h, 5 h and 15 h incubation (overnight) for microscopic analysis. After 15 h the cells treated with the composition of the present invention showed distinct morphological changes. The result is shown in FIG. 3. The transmission EM pictures show cells with a normal rod like structure in the control samples (FIG. 3A) and in samples only treated with the construct 11 alone (FIG. 3A, B), whereas the sample treated with the composition according to the present invention of construct 2 and construct 11 showed cells with drastically changed morphology which was club-shaped (FIG. 3 B). The altered appearance in form of a wider extension and less defined outer structure of the single mycobacteria. This altered, club-shaped morphology provides evidence that integrity of the mycobacteria is lost due to the treatment with the compositions of the present invention. Thus, the compositions of the present invention are able to degrade mycobacteria.

### EXAMPLE 4

### PCR-Test

40 μl of mycobacterial cells, with a cell number of about 1×10⁸, were incubated with 10 μl of the composition of first and second fusion protein solution of the present invention, 5 μl per fusion protein of the present invention was used, overnight at 24° C. The solution was centrifuged at 13000 rpm for 5 min to pellet the cell fragments and the intact cells, whereas the supernatant contains the released DNA and 10 μl of this supernatant were used as template for 50 μl PCR reactions, whereas the DNA was multiplied by the PCR. For the samples Taq-DNA Polymerase (Peqlab/Germany/Erlangen) and the primer pair 27f and 1492r, which was used at a 10 pM concentration, were used in a suitable reaction buffer. The given PCR Reaction protocol was performed in the Peqstar 2× gradient thermo cycler (Peqlab/Germany/Erlangen). The PCR reaction product was detected by Agarose-Gel-Electrophoresis and purified with the Qiagen Gel Extraction Kit (Qiagen/Germany/Hilden). Subsequently the purified DNA was sequenced with the primer 27f.

Used Primer Pair:
Primer 27f (190):

(SEQ ID NO: 119)

aga gtt tga tcc tgg ctc ag
Primer 1492r (191):

(SEQ ID NO: 120)

tac ggt tac ctt gtt acg act t

PCR Reaction Protocol:

| | | | |
|---|---|---|---|
| 95° C. | 2 | min | |
| 98° C. | 20 | sec | 15× (reducing 1° C./cycle) |
| 65° C. | 30 | sec | |
| 72° C. | l | min | |
| 98° C. | 20 | sec | 20× |
| 50° C. | 30 | sec | |
| 72° C. | l | min | |
| 72° C. | 5 | min | |

| | |
|---|---|
| 10° C. | ∞ |

The results of the PCR are shown in FIG. 4. FIG. 4 shows a picture of an agarose gel electrophoresis. With the above described PCR setting amplification of 16 S RNA PCL of the mycobacteria has been achieved. Thus, a strong signal of this 16 S RNA PCL in the agarose gel is a hint to a good degradation of the mycobacteria. As shown in FIG. 4, the exemplary used composition of the present invention of the first fusion protein PTD3-alpha defensin-Bxz2gp11-alpha defensin (Pat4 in FIG. 4) and the second fusion protein D29gp12-alpha defensin-PTD3 (Pat8 in FIG. 4) shows a good signal for 16 S RNA PCL (indicated with an arrow). This signal is stronger compared to the D29gp10 Lysin A-type endolysin alone. Therefore, the results of FIG. 4 show that the composition according the invention allows a good lysis of mycobacteria. Further, the compositions of the invention thus show to be useful for further diagnosis of mycobacteria due their ability to degrade mycobacteria in such an effective way.

### EXAMPLE 5

### Ability of the Compositions of First and Second Fusion Protein According to the Invention to Degrade Intracellular Mycobacteria

M. smegmatis strains were grown in Middlebrook's 7H9 broth medium supplemented with 10% OADC (Oleic acid-albumin-dextrose-catalase) and 0.05% Tween 80 at 37° C. on a shaker at 120 rpm. The mouse macrophage cell line RAW264.7 (described in: Membrane-active antimicrobial peptides and human placental lysosomal extracts are highly active against mycobacteria. Jena P, Mishra B, Leippe M, Hasilik A, Griffiths G, Sonawane A. Peptides. 2011 May; 32(5):881-7.doi: 10.1016/j.peptides.2011.03.002. Epub 2011 Mar. 17) was cultured in DMEM supplemented with 10% fetal calf serum (FCS), 1% penicillin-streptomycin solution, 1% 1-glutamine and HEPES. To investigate whether the compositions with the first and second fusion proteins according to the invention were able to kill intracellular M. smegmatis, 5×10⁵ RAW264.7 cells were infected with bacteria for 1 h at multiplicity of infection 10. Then extracellular bacteria were killed by addition of 10 μg/ml gentamicin. Infected macrophages were incubated with the compositions of first and second fusion proteins in DMEM medium for at least 4-8 h. After the incubation period, cells were washed, lysed with sterile H₂O and the intracellular survival was estimated by plating serial dilution of the cultures on 7H10 plates. Subsequently the colonies were enumerated after 72 h. The result whether lysis can be observed is shown in the following table 10:

TABLE 10

| Construct | lysis |
|---|---|
| Pat 2 | − |
| Pat 3 | − |
| Pat 8 | − |
| Pat 11 | − |
| Pat 2 + Pat 8 | ++ |
| Pat 2 + Pat 11 | + |
| Pat 3 + Pat 8 | ++ |

The results as shown above provide evidence that the compositions of the present invention comprising the first and second fusion protein are able to degrade also mycobacteria which are intracellular within the macrophages.

This provides evidence that the Protein Transduction Domain which is comprised within the first and second fusion protein of the composition of the present invention is able to deliver the first and second fusion protein through the eukaryotic cell membrane into the intracellular space. Further, the above results demonstrate that the first and second fusion proteins of the compositions of the present invention are still able to degrade mycobacteria after the passage through the eukaryotic membrane.

1. A composition having the activity of degrading the cell wall of a Mycobacterium species comprising:
(a) a first fusion protein comprising
(i) a first endolysin or a first domain, both having a first enzymatic activity, the enzymatic activity being at least one or more of the following: N-acetyl-b-D-muramidase (lysozyme, lytic transglycosylase), N-acetyl-b-D-glucosaminidase, N-acetylmuramoyl-L-alanine amidase, L-alanoyl-D-glutamate (LD) endopeptidase, c-D-glutamyl-meso-diaminopimelic acid (DL) peptidase, L-alanyl-D-iso-glutaminyl-meso-diaminopimelic acid (D-Ala-m-DAP) (DD) endopeptidase, or m-DAP-m-DAP (LD) endopeptidase;
(ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and
(iii) a protein transduction domain (PTD) being at the N- or C-terminus of the first fusion protein, wherein the PTD is having the characteristic to deliver a cargo from the extracellular to the intracellular space of a cell; and
(b) a second fusion protein comprising
(i) a second endolysin or a second domain, both having a second enzymatic activity, the enzymatic activity being at least one or more of the following: lipolytic activity, cutinase, mycolarabinogalactanesterase, or alpha/beta hydrolase;
(ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and
(iii) a protein transduction domain (PTD) being at the N- or C-terminus of the second fusion protein, wherein the PTD is having the characteristic to deliver a cargo from the extracellular to the intracellular space of a cell.

2. The composition according to claim 1, wherein the first endolysin is Lysin A (LysA) or the first enzymatic activity of the first domain is exerted by Lysin A (LysA) or Lysin A like enzymes and the second endolysin is Lysin B (LysB) or the second enzymatic activity of the second domain is exerted by Lysin B (LysB) or Lysin B like enzymes.

3. The composition according to claim 1 or 2, wherein the first and second enzymatic activity is exerted by enzymes derived from mycobacteriophages selected from the group consisting of TM4, D29, L5, and Bxz2.

4. The composition according to claim 1, wherein the AMP selected from the group consisting of cathelicidins (hCAP-18/LL37), alpha defensins, beta defensins, hepcidin, NK-2, and Ci-MAM-A24.

5. The composition according to claim 1, wherein the PTD is selected from the group consisting of TAT48-60, TAT47-57, TAT47-55, PTD3, PolyArginine, CADY, PepFect6, RXR, Antennapedia, Kala Syn, M918, MAP, Penetratin, PTD5-Syn, Pvec, Poly Arg 8, TAT 48-60, Transportan, Transportan10, and TAT-9.

6. The composition according to claim 1, wherein the Mycobacterium species is selected from the group consisting of Mycobacterium tuberculosis, Mycobacterium microti, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium abscessus, Mycobacterium kansasii, Mycobacterium terse, Mycobacterium nonchromogenicum, Mycobacterium gordonae, and Mycobacterium triviale.

7. The composition according to claim 1, wherein the endolysin or the domain having the first enzymatic activity of the first fusion protein exhibits an amino acid sequence selected from the group consisting of SEQ ID NO:1, 3, 5, and 7, wherein the endolysin or the domain having the second enzymatic activity of the second fusion protein exhibits an amino acid sequence selected from the group consisting SEQ ID NO:9, 11, 13, and 15, wherein the peptide stretch of the first and second fusion protein exhibits an amino acid sequence selected from the group consisting SEQ ID NO:17, 19, 21, 23, 25, and 27, and wherein the PTD exhibits an amino acid sequence selected from the group consisting SEQ ID NO:29, 30, 31, 32, 33, 34, 35, 36, 37, 38, 39, 40, 41, 42, 43, 44, 45, 46, 47, and 48.

8. The composition according to claim 1, wherein the first fusion protein exhibits an amino acid sequence selected from the group consisting SEQ ID NO:49, 51, 53, 55, 57, 59, 61, 77, 79, 81, 89, 91, 93, 95, 97, 99, 113, and 115, and wherein the second fusion protein exhibits an amino acid sequence selected from the group consisting SEQ ID NO:63, 65, 67, 69, 71, 73, 75, 83, 85, 87, 101, 103, 105, 107, 109, 111, and 117.

9. An isolated nucleic acid molecule encoding the first fusion protein of the composition according to claim 1.

10. A vector comprising a nucleic acid molecule according to claim 9.

11. A host cell comprising a nucleic acid molecule according to claim 9.

12. A feed or food additive comprising the composition according to claim 1.

13. A method for the treatment or prevention of a disease caused by an infection with a Mycobacterium species in a subject, such as tuberculosis, leprosy, pulmonary disease, lymphadenitis or skin disease comprising contacting said subject suffering or at risk of said disease with a composition according to claim 1.

14. A method for the treatment or prevention of a contamination caused by a Mycobacterium species of foodstuff, of food processing equipment, of a food processing plant, of a surface coming into contact with foodstuff, of a medical device, or a surface in a hospital or surgical suite comprising contacting said foodstuff, said equipment, said plant, said surfaces or said device with a composition according to claim 1.

15. The method according to claim 13, further comprising treating said subject with an additional therapeutic agent.

16. An isolated nucleic acid molecule encoding the second fusion protein of the composition according to claim 1.

17. A vector comprising a nucleic acid molecule according to claim 16.

18. A host cell comprising a nucleic acid according to claim 16.

19. A medicament or disinfectant comprising the composition according to claim 1.


SEQUENCE LISTING (120 sequences)

SEQ ID NO: 1 - protein, 547 aa, Artificial sequence
  TM4gp29 Lysin A
MSFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAA

SEQ ID NO: 2 - DNA, 1644 bp, Artificial sequence
  TM4gp29 Lysin A
atgagtttcacccggttcctgcaggatgacccgctgctcacccgcgagcaagtgatggccgagctgattcgggtcgccgacgagctgaacatgcccgacaagcgcggcgcctgcgtcattgcgggcatgacgatttcgcaagaggtcggcgtaaaggacaacgacccgccgttcgagcggcggttctggtgcccggccaaccgcgccgaccccgaatcgttcaactacccgcacgactcggaatcgaacgacggccgctcggtcggctacttccagcagcagaaggggcctaacggcgagctgtggtggggcacaacggcatccgagatgaacctgcacagcgccgcgacgcagtttatgacgcggctcaaggcggccggatacaacgcgagcaacgcccaggcggcgaacgactcggcgcaggcgatccagcggtcgggcgtcccgcaggcgtacaagcaatggtgggacgacattaaccgcctgtacgacaaggtgaagggctcgggcggtggcccggcgcccgcgcctaagccgccgcagtcggggccgtggaccggcgacccggtgtggctggccgacgtgctgcgcgccgaggggctgaacgtcgtcgagctgcccggctggctcgaccgcgggcacggcgacatgggccgcttgtggggcgtggtgtgccatcacaccggcagcgataacaccccgtcgagcgagattgcgtttcacccgtcgctcggcctgtgctcgcagattcacctggcgcgcaacggaactgtgacgctgtgcggtgtcggcatcgcctggcatgcgggcgtcggcagctatcccggcctgcccgaggacaacgccaacgcggtcactatcggcatcgaggcccaaaacagcggcacctatgacggcgcaccgcaccggacgaattggcctgacgcgcaatacgacgcctatgtgaagtgctgcgccgcgatctgccgccgcctcggcgtgcgcgccgatcacgtgatcagtcacaaggaatgggccgggcgcaagcaaggcaaatgggatccaggcgccatcgacatgaacatctttcgcgccgacgtacagcggcgcatcgacgcccatcaaccaaacggagaggacgatttcatggccgcactatcagccgacgagcagcgcgaggtgctgaacctgctgcgcgtcctggccgaccggcggttcgtcagccgcagcccgttccgccaccttggcgaggggccgagcgaaactgtcgccgggttcgggctcaacaccgacggcctcaatcacgcgcagtacacgattgagcttgcgcgcctgggcgacccgacgcacctcgccctgctgcgcgaggtcgccagcgccgagggtgactcgcgctatcccgaccggcagtacgacgccaagctcgccaagcgcgtgctcgccgaaatcgagggcgccgcaacggcaccggccaagccgagcacgccgagcgccccgaccgagcccgcccccgaggcgcccacgccgccggtcaaggccgcgtgtgcgctgtctgcggccgggtgcgtggtggctggctcgacctcgggcggtggctgcgccctgtccaccgacggcaccggcaagtgcgttgtgaccgccgcgaccgacggcggggccgcctga

SEQ ID NO: 3 - protein, 514 aa, Artificial sequence
  BXZ2gp11 Lysin A
MTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGEN

SEQ ID NO: 4 - DNA, 1545 bp, Artificial sequence
  Bxz2gp11 Lysin A
atgacggagaaggtacttccctacgaccgcagcatcgtcacgcaggagaccggctggtggtgtggccctgcggccacccaggtcgtgctcaactcgcgaggcatcatcgtcccggaggccacgctcgctgccgagatcgaggccatcgagaaccccggacggggtgacgaccgcgacggcaccgactacgtcggcctgatcgagcaggtgctggatcgccgtgtgccgcaggcgcgctacacgtcggtctacctgacgaacgatccgcccacgcaggctcagaaggaccggctgtgggagcacatcgtccggtcgatcaacgcgggctacggcgtggtcatgaactgggtcgcgcctccgtcgaacaagccacgcggagtgaagggctcggtgagcccgcgctactcgggcggcaccacgtaccactacgtcgcgtgcatgggctacgacgacacccccggtgctcgggcggtctggatcgccgacagcggcttccagccgcagggctactggatctcgttcgaccagtgcgccacgctgatcccgccgaagggctacgcgtacgccgacgccgcaccggctgctcccgcacccgcaccgacccccgtggtcgacgccgcgccgatcctggcgcgtgctgcgggcatctccgaggccaaggcccgcgagatcctgccgacgatgcgtgacgggctgaagcaggccgactgcaccaccgtcaaccggatcgcgatgttcatcgcccagaccggccacgagtccgacgacttccgggccaccgaggagtacgccaacggtcccctggaccaggagcgctggatctacaagggacgcacctggattcagatcacctggcgcgagcactacgcccggttcgggaagtggtgcttcgaccgcggcctggtgaccgaccccgacgtgttcgtcaagaacccgcgtgcgctggccgatctgaagtgggccggcatcggcgcggcctggtactggacggtcgagcgcccggacatcaacgcgctgtgcgaccgccgcgacatcgagacggtctcgcgacggatcaacgggacgaacccgaacaccggacgcgccaaccacatcgaagagcggatcgcccgctggaaccgcgcactcgcggtcggtgacgacctgctgcaacttatccgagaggaggaggacggcttcttgtccgcactcacacccgctgaacagcgcgctctctacaacgagatcatgaagaagggtccgacccggtcgttcatggccgaggaccagaaccagatcgagacgctgctcggcttcgtctacaacatcgacggcaacatctggaacgacgcggtgacccgcgcctacctgttcgacgtgccactggctgttgagtacgtcgagcgcgttgctcgcgacggcgtccacccgaagtcgtgggcgttccagcagctcgacggcaagggcgagcgctggctggccaagttcggccaggagtactgcaagggcctgatccgcttcaagaagaagctgaacgacctgcttgagccgtacggggagaactga

SEQ ID NO: 5 - protein, 493 aa, Artificial sequence
  D29gp10 Lysin A
MTLIVTRDHAQWVHDMCRARAGNRYGYGGAFTLNPRDTTDCSGLVLQTAAWYGGRKDWIGNRYGSTESFRLDHKIVYDLGFRRLPPGGVAALGFTPVMLVGLQHGGGGRYSHTACTLMTMDIPGGPVKVSQRGVDWESRGEVNGVGVFLYDGARAWNDPLFHDFWYLDAKLEDGPTQSVDAAEILARATGLAYNRAVALLPAVRDGLIQADCTNPNRIAMWLAQIGHESDDFKATAEYASGDAYDTRTDLGNTPEVDGDGRLYKGRSWIMITGKDNYRDFSRWAHGRGLVPTPDYFVVHPLELSELRWAGIGAAWYWTVERPDINALSDRRDLETVTRRINGGLTNLDDRRRRYNLALAVGDQLLTLIGDDDELADPTIQRFIREIHGALFNTVVTQSPYGDPQNPDGSEPRSNLWQLHELIKNGDGMGHARYVEESARAGDLRELERVVRAAKGLGRDRSPEFIARARNVLAQIEAANPEYLQAYIARNGAL

SEQ ID NO: 6 - DNA, 1482 bp, Artificial sequence
  D29gp10 Lysin A
atgacgctcatagtcacacgcgaccacgcgcagtgggtccacgacatgtgccgcgctcgcgctggcaacaggtacggctacggcggggcgttcacactcaacccccgagacaccaccgactgctcgggtctggttctgcagacggcagcctggtacggcggtcggaaggactggatcggaaaccggtacggctcgactgagagcttccggctcgaccacaagatcgtctacgacctcgggttcaggcgactccctccgggaggcgttgcggccctgggattcaccccggtcatgctcgtcgggctccagcacggcggcgggggccggtactcgcacaccgcttgcacgctgatgacgatggacatccccggtggcccggtgaaggtctcgcaacgaggcgtcgactgggagtcccgaggagaagtcaacggcgtgggggtgttcctctacgacggcgcacgcgcctggaacgacccgctcttccacgacttctggtacctggacgcgaagcttgaagacggcccgacgcagagtgtcgacgctgccgaaatcctcgctcgcgcaacgggtctcgcgtacaaccgagcggtagcactgctgccggccgtgcgtgacggcctcatccaggccgactgcaccaacccgaatcgcatcgcgatgtggctcgcccagatcggccatgagtcagacgatttcaaggccactgcggagtacgccagcggggacgcctacgacacccgaaccgacctcggcaacaccccggaggtcgacggagacggtcggctctacaagggccggtcctggatcatgatcacgggcaaggacaactaccgggacttctcccggtgggctcacggcaggggcctggtccccacgcccgactacttcgtggttcacccgctggagctgtcggagctgcgctgggcaggcatcggtgccgcctggtactggaccgtcgagcgcccagacatcaacgcactcagcgaccgccgcgacctcgaaacggtcacgcgccggatcaacggcgggctcaccaacctcgatgaccgccgacgccggtacaacctggccctcgctgtgggcgaccaactactgactctgatcggagatgacgacgaattggctgatccaacgattcagcggttcatccgcgagatccacggggcgctgttcaacaccgtcgtgacgcagtccccctacggcgacccgcagaacccggacggctcggagccccggagcaacctctggcagctccatgagctgatcaagaacggcgacggcatggggcacgcccgctacgtcgaggaatcggcgcgagccggtgacctccgcgagctggagcgagttgtccgcgccgccaagggacttggtagggatcgctcccccgagttcatcgcacgcgctcggaacgtgctggcccagatcgaggcagccaaccccgagtacctacaggcgtacatcgccaggaatggagccctatga

SEQ ID NO: 7 - protein, 292 aa, Artificial sequence
  L5gp10 Lysin A
MTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGL

SEQ ID NO: 8 - DNA, 879 bp, Artificial sequence
  L5gp10 Lysin A
atgaccttcacagtcacccgcgagagagcgcagtgggtccacgacatggcccgcgctcgcgacggtctcccctacgcgtacggcggggcgttcaccaacaacccgagggtgtcgactgactgctctggcctggtgctgcagaccggggcttggtatggaggtcgcaccgactgggtcggaaaccgttacggctcaaccgaatcgttccggctcgaccacaagatcgtctacgacctagggttcaagcggatgccccgaggcgggccagcggccttgccgatcaagccggtgatgctcgtcgggctccagcacggaggcggcggggtctactcgcacaccgcttgcacgttgatgacgatggaccaccccggtggcccggtcaagatgtccgaccgaggcgtcgactgggagtcccacggcaaccgcaacggcgtaggcgtcgaactttacgagggcgcacgggcatggaacgaccctctgttccatgacttttggtacctggacgcagtcctcgaagacgaaggagacgatgacgaattggctgacccagttctagggaagatgatccgcgagatccacgcgtgcctgttcaatcagaccgcgtcgaccagcgatctggcgacccctggtgaaggcgctatctggcagctacaccagaagatccactcgattgacggcatgctccacccgatccacgctgagcggcgcgctcgcgcaggcgatctcggtgagctgcaccgaatcgtgttggccgcgaagggcttgggcgtgaagcgcgacgaggtgaccaagcgggtctaccagagcatcctcgccgacatcgagcgggacaaccccgaagtacttcagcgatacatcgcagaaagaggtggcctatga

SEQ ID NO: 9 - protein, 400 aa, Artificial sequence
  TM4gp30 Lysin B
MAWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAA

SEQ ID NO: 10 - DNA, 1203 bp, Artificial sequence
  TM4gp30 Lysin B
atggcctgggtcggttggcagctcggcatgcagggggagcaggtcaaggtgatacagcaaaagctgatcgccaagtaccagtgggtgcgtgaccgttacccgcggctgacggccagcggcgtctatgacgtgaacacgcaggccgcgatcgtcgagtttcagttccgcgcagggcttcccgtcaccggcatagctgactatgcgacgcaggttcggctcggcgcggtggccccggcgccgccgccgcggcagcgcatcatggtgctgacgtttagcggcacctcggccgacatgtggaccggctatccggccgacgtcgcgcgtgcgctcgacccgtcgatcttctactggcagccagtgtgctacggccccaacggcatcccggcgatattcccgatgggttccagcgccaagagcggcgaggtcgaggggctgcggctgctcgacgagaaggcgcgcgatttcgactacatcgtgcttatcggatactcgcagggcgcgctgcccgcgtcgcggctcatgcggcgcatcctgtcgggcgacctgcagcggttcaagtccaagctgatcgccggtgtcacgttcggcaacccgatgcgcgagaaggggcacacgttccccggcggcgccgaccccggcgggcacggcctcgacccgcagtgcctcgtgaatacgcccgactggtggcacgactacgccgccaagggcgacatttacaccgtcggctcgggcagtaacgacgagaaggccaacgccgacatgacgttcatttaccagctcgtgcagggcgacattctcggcatgatgttcggcaccggcaacccgctcgacattctcggcctgctcggcggcctcggtggcggcctgctcggcggcctgggcggtggcctgctcggtggcggcaagggtggcctgcagttgccgagcggcctggtgctccccggcgtccagggcggcgcgctcaccgaccaccagcgcggcctcgtcgaggcggtgctggcgctgctcgctaacccgttcgccgaggttccggcggcggtcaaggcgattgtgtccggtgtcgggttcatcgccaccaacccgccgacggcgccgcacatcgagtaccacattcgcgaggctgcgcccggcgtgacgtatttccagcacgcgatcgactacctgcgccaggtcggcgcgtccgtcgccgctcgcgcggcctga

SEQ ID NO: 11 - protein, 321 aa, Artificial sequence
  Bxz2gp12 Lysin B
MPLRVGSNDANTGGLVSRWQKTMLARYAAYAKAYDGGPLRVDGYFGYDDADVQREYERRTHQVVDGEVSDADLRALGLEAAKRWLFTVHGTGQADPLGPGLPADTARAVLDKYTWQPIGNYPARAFPMWSSIMDGARELRSQIASKSGEVNLAGYSQGAVVVGQVLKHDIMDPKGSLHHRLGDVRKVVLWGNPMRQRGIAHFDEWIHPVAGPDSYGILDDRLEGLEKAPFEIRDYAHAGDMYASITDGDKDEYKIAICKIVMTATDFYRGPNSVVSQLIELGQRPLTEGIAMALAIIDTLRFFTNTAHGYNIGPAIDFLRS

SEQ ID NO: 12 - DNA, 966 bp, Artificial sequence
  Bxz2gp12 Lysin B
atgcccctgcgggtcgggtcgaacgacgccaataccggcgggctggtgagccgttggcagaagacgatgctggcccgctacgcggcctacgccaaggcgtacgacggcgggccgctgcgagtcgacggctatttcggctacgacgacgctgacgttcagcgcgagtacgaacgccgcacccaccaggtggtcgacggtgaggtcagtgacgccgacctccgggctctgggcctggaggcggcgaagcgctggctgttcacggtccacggcaccggacaggccgacccgctgggtccgggactccccgccgacacggcgcgggcggtgctcgacaagtacacctggcagcccatcggcaactaccccgctcgggcgttcccgatgtggtcctcgatcatggacggtgccagggagcttcgctcccagatcgcgtcaaagtccggtgaggtcaacctggcgggctactcgcaaggcgcggtggtcgtcggccaggtgctcaagcacgacatcatggacccgaagggcagcctgcaccacaggctcggcgatgtccgcaaggtagtgctctggggaaatcccatgcgccagaggggaatcgctcacttcgatgagtggattcacccggtggcaggcccagactcgtacggcatcctcgatgaccggctcgaagggctggagaaggcaccgttcgagatccgggactacgcgcacgctggtgacatgtacgcctccatcacggacggcgacaaggacgagtacaagatcgcgatctgcaagatcgtcatgacggcgacggacttctaccgaggcccgaactccgttgtgtcccaactgatcgagcttggacagcgtccgctcaccgagggcatcgcaatggccctggcgatcatcgacacgctgcggttcttcacgaacaccgcgcacggctacaacatcggaccagctatcgacttcctgcgtagctga

SEQ ID NO: 13 - protein, 254 aa, Artificial sequence
  D29gp12 Lysin B
MSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 14 - DNA, 765 bp, Artificial sequence
  D29gp12 Lysin B
atgagcaagccctggctgttcaccgttcacggcacgggccagcccgatcccctcgggcctggcctgcctgccgatacggcacgcgacgtacttgacatctaccggtggcagcccatcggcaactaccccgctgcggccttcccgatgtggccgtcggtcgagaagggtgtcgccgagctgatcctgcagatcgagctgaagctggacgcggacccctacgcggacttcgcgatggcgggttactcgcagggagccatcgtggttggccaggtgctcaagcaccacatcctgcctccgacgggcaggctccacaggttcctgcaccggctcaagaaggtcatcttctggggtaatcccatgcggcagaagggctttgcccactctgacgagtggatccacccggtcgctgcccctgacaccctcggaatcctcgaggaccggctcgaaaacctggagcagtacggcttcgaggtccgcgactacgcccacgacggtgacatgtacgcctccatcaaagaggacgacctgcacgaatacgaggtcgccatcggccggatcgtgatgaaggccagcggcttcatcggtggccgggactccgtggtagcccagctcatcgagcttggccagcgtccgatcaccgagggaattgcgttggcgggagccatcatcgacgccctcacgttcttcgcccgctctcgtatgggcgacaagtggccgcacctctacaaccgctacccggcggtcgagttcctacgacagatctga

SEQ ID NO: 15 - protein, 254 aa, Artificial sequence
  L5gp12 Lysin B
MSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRI

SEQ ID NO: 16 - DNA, 765 bp, Artificial sequence
  L5gp12 Lysin B
atgagcaagccctggctgttcaccgtccacggcacaggccagcccgacccgctcgggcctggtctgcctgccgataccgcacgggacgtacttgacatctaccggtggcagcccatcggcaactacccggcagcggcgttcccgatgtggccgtcggtcgaaaagggtgtcgctgagctgatcctgcagatcgagctgaagctggacgcagatccgtacgcggacttcgcgctggccggctactcgcagggagccatcgtggtgggccaggtgctcaagcaccacatcatcaacccgagaggtcgactgcaccggttcctgcaccggctcaggaaggtcatcttctggggtaatccgatgcggcagaagggctttgcccacaccgacgagtggattcaccaggtcgctgcctcggacacgatgggcatcctcgaggaccgactggagaacctcgagcagtacggctttgaggtccgcgactacgcgcacgacggcgacatgtacgcctccatcaaggaggacgacatgcacgagtacgaggtggccattggccgaatcgtgatgagcgctaggcgattcatcggaggtaaggactccgtcatcgcccagctcatcgagcttggacagcgtccgatctgggagggaatcgcgatggccagagccatcatcgacgccctcacgttcttcgccaagtcgacccaaggcccgagctggccgcatttgtacaaccgcttcccggcggtcgagttcctacgacgaatctga

SEQ ID NO: 17 - protein, 37 aa, Artificial sequence
  LL-37
LLGDFFRKSKEKIGKEFKRIVQRIKDFLRNLVPRTES

SEQ ID NO: 18 - DNA, 111 bp, Artificial sequence
  LL-37
ttattgggtgatttctttcggaagagcaaagaaaagataggaaaggagtttaaacgaattgttcaacgtatcaaagacttcctaaggaatcttgtaccaagaacagaaagt

SEQ ID NO: 19 - protein, 30 aa, Artificial sequence
  alpha-defensin
DCYCRIPACIAGERRYGTCIYQGRLWAFCC

SEQ ID NO: 20 - DNA, 90 bp, Artificial sequence
  alpha-defensin
gattgttattgtagaataccagcatgcattgcgggagaacgtaggtacggaacatgcatctatcaaggtcgattatgggctttttgttgc

SEQ ID NO: 21 - protein, 38 aa, Artificial sequence
  beta-defensin
NPVSCVRNKGICVPIRCPGSMKQIGTCVGRAVKCCRKK

SEQ ID NO: 22 - DNA, 114 bp, Artificial sequence
  beta-defensin
aatccagtaagctgtgttcgtaataagggtatttgcgttccaatacgatgcccaggaagtatgaaacaaatcggtacatgcgtaggaagagcagtaaagtgttgtaggaaaaaa

SEQ ID NO: 23 - protein, 25 aa, Artificial sequence
  Hepcidin
DTHFPICIFCCGCCHRSKCGMCCKT

SEQ ID NO: 24 - DNA, 75 bp, Artificial sequence
  Hepcidin
gatacacactttccaatatgcattttctgttgcggttgctgtcatagaagtaaatgtggaatgtgctgtaagaca

SEQ ID NO: 25 - protein, 27 aa, Artificial sequence
  NK-2
KILRGVCKKIMRTFLRRISKDILTGKK

SEQ ID NO: 26 - DNA, 81 bp, Artificial sequence
  NK-2
aaaatcttacgaggtgtatgtaaaaagattatgagaacatttttgcgtaggataagtaaagatatactaacaggaaagaag

SEQ ID NO: 27 - protein, 24 aa, Artificial sequence
  Ci-MAM-A24
WRSLGRTLLRLSHALKPLARRSGW

SEQ ID NO: 28 - DNA, 72 bp, Artificial sequence
  Ci-MAM-A24
tggcgaagtttaggaagaacactacttcggttgagccatgcattgaaaccattagctaggcgtagtggttgg

SEQ ID NO: 29 - protein, 14 aa, Artificial sequence
  TAT48-60
GRKKRRQRRRPPQC

SEQ ID NO: 30 - protein, 11 aa, Artificial sequence
  TAT47-57
YGRKKRRQRRR

SEQ ID NO: 31 - protein, 9 aa, Artificial sequence
  TAT47-55
YGRKKRRQR

SEQ ID NO: 32 - protein, 11 aa, Artificial sequence
  PTD3
YARKARRQARR

SEQ ID NO: 33 - protein, 9 aa, Artificial sequence
  PolyArginine
RRRRRRRRR

SEQ ID NO: 34 - protein, 20 aa, Artificial sequence
  CADY
GLWRALWRLLRSLWRLLWRA

SEQ ID NO: 35 - protein, 21 aa, Artificial sequence
  PepFect6
AGYLLGKINLKALAALAKKIL

SEQ ID NO: 36 - protein, 14 aa, Artificial sequence
  RXR
RXRRXRRXRRXRXB

SEQ ID NO: 37 - protein, 16 aa, Artificial sequence
  Antennapedia
RQIKIWFQNRRMKWKK

SEQ ID NO: 38 - protein, 30 aa, Artificial sequence
  Kala Syn
WEAKLAKALAKALAKHLAKALAKALKACEA

SEQ ID NO: 39 - protein, 22 aa, Artificial sequence
  M918
MVTVLFRRLRIRRASGPPRVRV

SEQ ID NO: 40 - protein, 18 aa, Artificial sequence
  MAP
KLALKLALKALKAALKLA

SEQ ID NO: 41 - protein, 16 aa, Artificial sequence
  Penetratin
RQIKIWFQNRRMKWKK

SEQ ID NO: 42 - protein, 12 aa, Artificial sequence
  PTD5-Syn
RRQRRTSKLMKR

SEQ ID NO: 43 - protein, 18 aa, Artificial sequence
  Pvec
LLIILRRRIRKQAHAHSK

SEQ ID NO: 44 - protein, 8 aa, Artificial sequence
  Poly Arg 8
RRRRRRRR

SEQ ID NO: 45 - protein, 13 aa, Artificial sequence
  TAT48-60
GRKKRRQRRRPPQ

SEQ ID NO: 46 - protein, 27 aa, Artificial sequence
  Transportan
GWTLNSAGYLLGKINLKALAALAKKIL

SEQ ID NO: 47 - protein, 21 aa, Artificial sequence
  Transportan10
AGYLLGKINLKALAALAKKIL

SEQ ID NO: 48 - protein, 9 aa, Artificial sequence
  TAT-9
RKKRRQRRR

SEQ ID NO: 49 - protein, 603 aa, Artificial sequence
  TM4gp29/LL-37/TAT47-57
MSFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAAGSGSLLGDFFRKSKEKIGKEFKRIVQRIKDFLRNLVPRTESGAGAYGRKKRRQRRR

SEQ ID NO: 50 - DNA, 1809 bp, Artificial sequence
  TM4gp29/LL-37/TAT47-57
atgagtttcacacgttttttacaagatgacccactattaacacgtgagcaagttatggcggagttaatacgggttgctgacgaacttaatatgccagacaagcgtggagcgtgcgtaatagctggaatgacaattagccaagaggttggtgttaaagacaatgatccaccatttgagaggcgtttctggtgcccagctaatcgtgccgatccagaaagtttcaattatccacacgatagcgaaagcaatgatggtaggagcgttggatattttcaacaacaaaagggtccaaatggagaattgtggtggggtacaacagctagcgaaatgaatttacacagtgcagcgacacaattcatgacaaggctaaaggcggctggatacaatgccagtaatgcgcaagccgctaatgatagcgcacaagccatacaacgaagcggtgttccacaagcatataagcaatggtgggatgacataaatcggctttatgacaaagtaaagggaagcggtggaggtccagcgccagccccaaaaccaccacaaagcggtccatggacaggagatccagtatggttagccgatgtactaagggcagaaggattgaatgttgtagaattaccaggttggctagaccgaggacatggtgatatgggacggttgtggggtgttgtatgtcatcacacaggaagcgataatacaccaagcagtgaaatcgcctttcatccaagccttggattatgcagccaaatacatttggccagaaatggaacagttacattgtgtggagttggtattgcatggcacgcaggtgttggtagttatccaggtctaccagaggacaatgcaaatgctgttacaattggtattgaagctcaaaatagcggaacatatgacggtgcgccacatcggacaaattggccagacgcgcaatacgatgcttacgttaaatgctgtgcagctatctgtcggcgattgggagtaagggcggaccatgtaatcagtcacaaagaatgggcaggtaggaaacaaggtaaatgggacccaggtgcaatagatatgaatatatttagagccgatgtacaacgtcggatcgatgcccatcaaccaaatggagaagacgattttatggcggctttgagcgcagacgaacaacgagaggtattgaatcttttgcgtgtactagccgatcggagattcgtaagtaggagtccattcaggcaccttggagaaggtccaagcgaaacagtagcaggatttggtcttaatacagacggattgaatcatgcccaatatacaattgaacttgcccgattaggtgacccaacacacttggcacttttaagagaggtagcgagtgcggaaggtgacagcagatacccagatcggcaatacgacgccaagcttgcgaaaagggttctagcagaaattgaaggagcagccacagcgccagctaagccaagtacaccaagtgcgccaacagagccagccccagaagctccaacaccaccagttaaggctgcgtgcgccttaagtgcggctggatgtgttgtagcgggtagtacaagtggtggaggttgcgcactaagtacagatggtacaggaaagtgtgtagttacagccgctacagacggtggagcagctggaagtggaagtcttttaggagacttctttcgtaagagcaaagagaaaatcggaaaagaatttaaacgtatcgtacaacgaattaaggatttcctaagaaatctagtaccacgaacagagagtggtgcaggagcttacggtagaaagaaacggcgacaaagacgaaga

SEQ ID NO: 51 - protein, 595 aa, Artificial sequence
  TM4gp29/LL-37/TAT47-57
MSFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAALLGDFFRKSKEKIGKEFKRIVQRIKDFLRNLVPRTESYGRKKRRQRRR

SEQ ID NO: 52 - DNA, 1785 bp, Artificial sequence
  TM4gp29/LL-37/TAT47-57
atgagttttacaaggttcctacaagatgacccactattgacacgagaacaagtaatggctgagctaatacgagtagcagatgaattaaatatgccagataagaggggagcttgtgttatcgccggtatgacaatcagtcaagaagttggagttaaggacaatgatccaccattcgagcgtaggttctggtgtccagcgaatcgtgctgatccagaaagttttaattacccacacgacagcgaaagcaatgacggacgtagtgtaggttatttccaacaacaaaagggaccaaatggagagctttggtggggtacaacagctagtgagatgaatctacatagtgcagcgacacaatttatgacaagacttaaagctgccggttataatgccagtaatgcgcaagcggctaatgacagtgcacaagctatacaacgtagtggtgtaccacaagcctataagcaatggtgggatgacataaataggttatacgataaggtaaagggtagtggtggaggtccagcaccagcaccaaaaccaccacaaagcggtccatggacaggagatccagtttggttggcggacgtattacgagcagaaggtttgaatgtagttgaacttccaggatggcttgacagaggacacggtgacatgggacgtctttggggtgtagtttgtcaccatacaggtagtgacaatacaccaagtagcgaaattgcctttcacccaagtttgggactttgcagccaaattcatcttgccaggaatggtacagttacactatgcggagtaggaatagcttggcacgccggtgttggtagctatccaggtttaccagaagataatgctaatgcagtaacaattggaattgaagcacaaaatagcggaacatacgacggtgcaccacatagaacaaattggccagacgctcaatacgatgcgtatgttaaatgctgtgctgccatatgccggcgattaggtgtacgggctgaccatgttattagccacaaggaatgggccggaagaaaacaaggaaaatgggacccaggagctatcgatatgaatatctttcgggcggatgtacaacggcgaattgatgctcatcaaccaaatggagaagatgactttatggcggccttaagtgccgacgaacaacgtgaggttttaaatcttttacgggtattggcggaccggcgattcgttagtaggagcccattccgacatttgggtgagggaccaagcgagacagtagcgggattcggtttgaatacagatggtttgaatcatgcacaatatacaatagaattggcaagattgggtgacccaacacacctagcgcttttaagggaagtagcaagtgcagaaggtgatagccgttatccagatcgtcaatacgatgccaagctagcgaaacgagttttagcagaaattgaaggagccgcgacagcgccagccaaaccaagcacaccaagtgctccaacagaaccagcaccagaggcgccaacaccaccagttaaagctgcctgtgccctaagtgcagcgggatgcgtagttgccggaagcacaagcggaggtggatgcgctttgagcacagacggaacaggtaaatgtgtagttacagctgcgacagatggtggagcagcattactaggtgactttttcaggaaaagcaaagaaaagatcggtaaagagtttaaaagaatagtacaacggatcaaggattttcttagaaatctagttccacggacagagagctacggaagaaagaagcggagacaaaggcgacgt

SEQ ID NO: 53 - protein, 555 aa, Artificial sequence
  Bzx2gp11/alpha-defensin/PTD3
MTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGENDCYCRIPACIAGERRYGTCIYQGRLWAFCCYARKARRQARR

SEQ ID NO: 54 - DNA, 1665 bp, Artificial sequence
  Bzx2gp11/alpha-defensin/PTD3
atgacagagaaagttttaccatacgatcggagtatagttacacaagagacaggttggtggtgtggtccagccgcaacacaagtagttttaaatagtcgtggaataatcgtaccagaagctacattggccgctgaaatcgaagccatagaaaatccaggtcgtggtgatgaccgagacggaacagattatgtaggtcttattgagcaagttcttgatcggagggttccacaagctcgttacacaagcgtatatttaacaaatgatccaccaacacaagcacaaaaggaccgattgtgggaacatattgtaagaagcataaatgcaggttacggagtagttatgaattgggtagctccaccaagtaataagccacgtggagttaaaggtagcgttagcccacgatatagtggaggtacaacatatcactatgtagcgtgcatgggatatgacgatacaccaggtgccagagcagtatggatagcggacagcggattccaaccacaaggatactggatcagctttgaccaatgcgccacacttatcccaccaaaaggttatgcatacgcagacgcggcaccagctgcaccagctccagctccaacaccagttgtagacgccgcgccaatattggcccgggcggcaggtattagcgaagcaaaggcaagggaaattcttccaacaatgcgggatggactaaaacaagcggattgtacaacagttaatcgaattgcaatgtttatagcccaaacaggacacgaaagtgatgactttcgggctacagaagagtatgcaaatggaccattggatcaagaacgttggatttacaaaggtcgaacatggatacaaattacatggagggaacactacgctcgtttcggtaaatggtgtttcgaccgtggacttgttacagatccagatgtatttgttaaaaatccacgagctttggccgacctaaaatgggcgggtataggagcggcttggtactggacagtagaacggccagatatcaatgccttatgcgatcgaagagacatcgaaacagttagtagaaggataaatggaacaaatccaaatacaggtagagcgaatcatatcgaggaacgaattgcgaggtggaatcgtgcactagcagtaggtgacgatttacttcaactaattagagaagaggaagacggttttttaagtgctttgacaccagccgagcaacgagcgttgtataatgaaatcatgaagaaaggaccaacacgtagtttcatggctgaggatcaaaatcaaatcgagacattattgggattcgtatacaatatagacggaaatatctggaatgacgctgtaacacgggcttatttatttgatgttccattagccgtagaatacgttgagagggttgcgagggatggagtacatccaaagagctgggcatttcaacaactagacggaaagggagaaaggtggctagccaagttcggacaagaatactgtaaaggtctaatccggttcaagaaaaagttgaatgatctacttgaaccatatggagagaatgactgctactgtcggattccagcgtgcattgctggtgaaaggagatatggtacatgtatatatcaaggtagactttgggccttttgttgctacgcgagaaaggccagaaggcaagcgcgtcga

SEQ ID NO: 55 - protein, 555 aa, Artificial sequence
  PTD3/alpha-defensin/Bzx2gp11
YARKARRQARRDCYCRIPACIAGERRYGTCIYQGRLWAFCCMTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGEN

SEQ ID NO: 56 - DNA, 1665 bp, Artificial sequence
  PTD3/alpha-defensin/Bzx2gp11
tatgcacgcaaagcgagacgacaagcccgtcgcgactgctattgtcgcatccctgcatgtatagcaggtgagcgtcggtacgggacatgcatttatcaaggtaggttatgggctttttgttgcatgacggagaaggttttgccttacgatcgtagtatagttacccaggaaaccggctggtggtgtggaccagcagccacgcaggtggtcttgaatagcaggggcatcattgtgccagaggcgactcttgccgctgaaattgaggctatcgagaatccagggaggggagatgacagagatggtactgattacgtaggcttaatagaacaggtccttgatcgacgtgtgcctcaagctaggtatacgtctgtttatttaacaaacgatcccccaactcaagcgcagaaagaccgcttatgggaacacatagtacggagcatcaatgcgggttacggcgttgtcatgaactgggtggccccaccttctaacaaaccccgtggggtcaagggttccgtatctccccggtattcaggcggaactacatatcattatgtcgcttgcatgggttacgacgatacaccgggagccagagctgtatggatagcagattccggattccaacctcagggctattggattagtttcgaccagtgcgctacgttaataccgcctaagggatatgcctacgcagacgccgctccggcggctcccgcaccagcaccgacgccggtagttgacgccgcaccgattttggcgagggctgcggggataagtgaagcgaaagcacgagagatcttacccactatgcgagacggccttaagcaggcggactgcacaacggtaaacaggatcgcgatgtttatagctcaaaccgggcatgaaagcgatgacttcagggcaaccgaggaatacgctaatggtccacttgatcaagagcggtggatttacaaaggcagaacctggatacaaattacgtggcgcgagcactacgcccgatttgggaagtggtgttttgatcggggtctcgtcacagatccagatgtgtttgtgaaaaaccccagagctttggcagacctcaaatgggcggggatcggcgcagcctggtactggactgtcgagcgtccggatattaatgcgctgtgtgacagacgagatattgagaccgtctcgcgacggatcaatggaacaaatcccaatacagggcgcgctaaccatattgaagagcgcatagcccggtggaacagagctctggcagttggagacgatctcctgcaactgatccgcgaggaagaggacgggtttctgtcagctctaaccccagcggaacaaagagcgctttacaacgagatcatgaagaaagggcctactcgttcattcatggccgaagaccagaatcagatagaaacgctattgggctttgtatataacattgacggtaatatctggaacgatgccgtaactcgggcgtatctattcgacgtgccgctagccgttgaatacgtggaaagagttgcacgagacggagtacaccctaagtcgtgggcattccaacagctcgatggtaaaggagagaggtggctagccaaattcggtcaggaatactgtaagggacttatacgttttaagaaaaagctcaacgatttgctagaaccctatggggaaaat

SEQ ID NO: 57 - protein, 585 aa, Artificial sequence
  PTD3/alpha-defensin/bzx2gp11/alpha-defensin
YARKARRQARRDCYCRIPACIAGERRYGTCIYQGRLWAFCCMTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGENDCYCRIPACIAGERRYGTCIYQGRLWAFCC

SEQ ID NO: 58 - DNA, 1755 bp, Artificial sequence
  PTD3/alpha-defensin/Bzx2gp11/alpha-defensin
tatgctcgtaaggcgcgccgacaggctcggcgcgactgttattgtcggatcccggcgtgcattgccggcgagcgcaggtacggtacgtgtatctaccagggacggttatgggcattttgctgtatgaccgaaaaggtcctaccatacgacagaagcatcgtgacacaggagaccggttggtggtgtggtcctgcggctacacaggtggtccttaattctcgaggcattatagtgccggaggccactcttgccgctgaaatagaagccattgaaaatcccggccggggagatgaccgggacgggacggactacgttgggttgattgaacaagtattggatcgccgtgtaccgcaagcgagatatacgagtgtatacctgactaacgatcccccgacgcaagcacaaaaagatcgcctctgggaacatatagttaggtccataaatgcgggttatggagtcgttatgaactgggtagcgccgccaagtaataaaccacgaggagtcaaaggtagtgtgtcacctcgatactcaggcgggactacgtatcactatgtagcttgtatgggctacgacgatacaccaggcgcaagggcggtttggatagcagattcgggcttccaacctcaaggatattggatctcgtttgatcaatgcgcaacccttattccgccaaagggatatgcgtacgcagacgctgcgcccgcggcaccagctccggcccctactcctgttgtagacgcagcccctatattagcacgtgcagccgggatctccgaggcaaaggctcgtgagatcttgcccactatgcgagacgggctaaaacaggcagactgcacaaccgttaatagaatagccatgttcatagcccaaaccggtcatgagagcgacgattttcgggccacggaggaatacgctaacggtcccctggaccaggaaaggtggatctataaggggagaacttggatacagattacctggagggagcattatgcgcgtttcggcaagtggtgcttcgaccgaggtttggtcaccgaccctgacgtctttgttaaaaacccacgcgcactcgcggacttgaaatgggctggaattggggcagcttggtattggaccgtagaaagacctgatatcaacgccctgtgcgaccggagagatattgagacagtatcaaggcgcatcaatggaacgaacccaaacactgggagggccaaccacattgaagagcgtatcgctagatggaatcgtgccctggccgtgggcgatgacctattacaactcattcgcgaagaggaagatggttttcttagcgcgctcacgcctgctgaacaaagagctctttacaacgagataatgaagaaaggacccacacgatcttttatggcagaggaccaaaaccagatcgaaacactattaggtttcgtctataatatagatgggaatatatggaatgatgcagtgactcgtgcttatttattcgatgtacccttggccgtggagtacgtcgagagggtggcgcgggatggagttcaccccaagtcttgggcctttcaacagctggatgggaagggcgagcgttggctagcaaaattcggtcaggaatactgcaaaggtttaatccggtttaaaaagaaactgaacgatctacttgagccatacggcgaaaatgattgttattgtagaataccggcgtgcattgcgggggaacgaaggtacggaacatgtatttatcagggacgactctgggctttctgctgt

SEQ ID NO: 59 - protein, 341 aa, Artificial sequence
  beta-defensin/L5gp10/TAT47-57
NPVSCVRNKGICVPIRCPGSMKQIGTCVGRAVKCCRKKMTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGLYGRKKRRQRRR

SEQ ID NO: 60 - DNA, 1023 bp, Artificial sequence
  beta-defensin/L5gp10/TAT47-57
aacccggtcagctgtgtcaggaacaagggaatatgtgttcccattcgctgcccggggagcatgaaacaaattggtacatgcgtagggcgtgcggtgaagtgctgtcgaaagaaaatgacctttactgtgacacgggaaagggctcagtgggtccatgatatggcgcgcgctagggacggtctaccttatgcatatggaggggcgtttacgaataacccacgcgtttcgacagactgttcgggcctggtcctgcagactggagcatggtatggtggccgaacagattgggttgggaatcgatatgggtctacggagtcatttagactcgaccataaaattgtatatgatttaggttttaaacgtatgcctcgggggggaccggcagctctgcccatcaagccagtaatgttagtaggtctacaacacggcggtggaggggtgtactcccatactgcgtgcacgttaatgaccatggaccatccaggcgggccagtgaaaatgtctgatcgtggagtagactgggaaagtcatgggaaccgcaacggagttggcgttgaactgtacgagggcgcccgggcttggaatgatcccttgttccacgatttctggtacttggatgccgtattggaggatgagggtgacgatgacgagctagccgatccggtccttggcaaaatgatacgagagatccacgcctgtttattcaatcaaaccgcttccactagtgatttggcgacccctggcgaaggtgccatatggcagctccatcagaagatccactcaatcgacggtatgcttcaccctatccacgccgagagaagggcacgcgcaggagacttaggagaactacatagaatagtgctcgctgcgaaagggcttggcgttaagcgggacgaggtcacgaagcgtgtataccaaagtattttggcagatattgaaagagacaatcccgaagtgcttcagagatacatagcagaaaggggaggcctctatggtagaaagaaacgaaggcaacgtcgacgg

SEQ ID NO: 61 - protein, 370 aa, Artificial sequence
  alpha-defensin/Hepcidin/L5gp10/TAT47-57
NPVSCVRNKGICVPIRCPGSMKQIGTCVGRAVKCCRKKGAGADTHFPICIFCCGCCHRSKCGMCCKTMTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGLYGRKKRRQRRR

SEQ ID NO: 62 - DNA, 1110 bp, Artificial sequence
  alpha-defensin/Hepcidin/L5gp10/TAT47-57
aatcctgttagctgcgttcgcaataaaggcatctgtgtgccaatacgatgtccaggtagtatgaaacaaatcggaacatgcgtgggaagagccgttaagtgttgccggaagaaaggagctggtgcggatacacacttcccaatatgcattttctgttgcgggtgttgccatcgttctaaatgtggtatgtgctgtaagaccatgacatttacggtaacacgggagcgagcccaatgggtacatgacatggcgcgcgccagagatggattaccgtacgcatacggcggtgcatttacaaacaatccgagggtatccaccgactgttctgggctcgtattacaaacgggcgcgtggtacggtggcaggacggattgggtagggaaccggtatggaagcactgagtcgtttagattggaccacaagatagtgtatgacctcggctttaaaagaatgccccggggaggccctgctgcgttacccatcaagccagtcatgttagttgggctacagcacggtggaggcggggtctattcacacacggcttgcactttaatgactatggaccatcccgggggccccgttaagatgagcgaccgtggggtcgattgggagtcccatggaaatcgcaacggggtaggtgtggaattgtatgaaggcgcaagagcctggaacgacccgctcttccatgatttctggtatcttgatgcggttttggaagacgagggcgatgacgatgaactagctgatccagtgctgggtaagatgatacgtgaaatccatgcatgtctatttaaccagactgcatcgacctcagacctcgcgactcctggagagggagcaatatggcaactgcaccagaaaatccacagtattgatgggatgctgcaccctattcatgcggaacgtagggctagggccggtgatttgggtgagctacatcgaattgtcttggctgccaaagggcttggagtcaagcgggacgaggtgaccaaacgcgtataccagagtattctggcagatattgaaagggacaatccggaagtccttcaacgatatatagctgagcgtggcggtctttacgggcgaaagaaacgtaggcagcgccgaaga

SEQ ID NO: 63 - protein, 456 aa, Artificial sequence [sequence omitted]

SEQ ID NO: 64 - DNA, 1368 bp, Artificial sequence
  TM4gp30/LL-37/TAT47-57
atggcttgggtcggctggcagcttggtatgcagggtgaacaggttaaggtaatccagcaaaagttgatagccaagtaccagtgggtgcgtgatagatatccccggttgaccgcaagtggggtttacgacgtcaacacccaagcagcgattgttgagtttcaatttagagccggacttccagtcacgggcatagccgactatgcgacacaggtgcggcttggagctgtcgcaccagctccaccccctaggcaacggattatggtactcacatttagcgggacgtcggctgatatgtggacaggctacccggctgacgttgcaagagccctagatccttcaattttttattggcaacccgtttgttatggcccaaacggtattcccgcgatattcccgatggggtcgagtgccaagtccggggaagttgaaggcttgcgactattagacgagaaagctcgcgatttcgactacattgtcctgatcgggtatagtcagggtgcgttgcctgcgagcaggttgatgcgtcgcatcctatcaggtgatctgcaaaggttcaaatcaaaactgatagcaggagtgacgtttgggaacccgatgcgagaaaaaggtcacacctttcctggcggggctgacccgggtggccacggcttagacccacagtgcctcgtgaatactccggattggtggcacgactacgcggccaaaggcgatatctacacagtaggttctggatctaatgatgagaaggccaatgcggacatgactttcatatatcaattagtccaaggagacatactcggtatgatgttcggtacgggtaaccctttggatatattagggttgctgggaggcctaggcggaggtctacttggaggcctaggtgggggcttgcttggaggtggaaaggggggattacaactgcctagcggacttgttttacctggggttcaggggggtgcattaaccgaccatcaacgtggacttgtagaagcggtgctggctttactcgcaaatcccttcgcggaagtacccgccgcggtgaaggcaatcgtaagtggcgtcggtttcatcgccactaatccgccaactgccccacatattgagtaccatattagggaggcagctcccggcgtgacatatttccaacatgcaatcgattatctcaggcaagtcggagcctccgtggctgcacgggctgcgggatctggatcactactgggagattttttccgaaagtctaaagagaaaatagggaaagaatttaagcgaattgtacagcgcatcaaggattttctacgaaacctcgtaccgcgtactgagagcggggcaggggcttacggtcgcaaaaaacgccggcagagacgtaga

SEQ ID NO: 65 - protein, 448 aa, Artificial sequence
  TM4gp30/LL-37/TAT47-57
MAWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAALLGDFFRKSKEKIGKEFKRIVQRIKDFLRNLVPRTESYGRKKRRQRRR

SEQ ID NO: 66 - DNA, 1344 bp, Artificial sequence
  TM4gp30/LL-37/TAT47-57
atggcctgggttggctggcagttaggaatgcagggtgaacaggtgaaggtaatacagcaaaaacttatagccaagtaccagtgggtcagggatagatatccccgcctaaccgcgagtggggtttacgacgtgaatactcaggcagctatcgttgagttccaatttagagctggcctaccagtaactggtatagcagactatgctactcaagtgcggttaggggcggtcgcgcctgctccgccaccccgtcaacgaatcatggtactgacattttcaggcacatcagcggatatgtggactggatatcccgcagacgtcgcgagggctcttgacccctcaatattctattggcagcccgtgtgctacgggcccaacggaattccggccatattccctatggggtcctctgcaaaatctggcgaggttgagggtttgcgtctactggacgaaaaagcccgagactttgattacatagtgctgataggttatagtcaaggagcgctcccggcgagtaggttaatgcggcgaattctgagcggcgatctccaacggtttaaaagcaagcttatcgcaggtgtaacctttgggaatcctatgcgggagaagggacatacatttcctggaggtgccgacccaggtggacacggcttagatcctcaatgtctcgtcaataccccggattggtggcacgattacgccgcgaagggagatatctacacagttggaagcggtagcaatgacgaaaaagctaacgcggacatgacatttatttatcagctggtgcagggggacatcctaggcatgatgttcggaacgggtaaccctctggatatattgggtcttctcgggggcttaggcgggggcttattgggcggtctagggggcggtcttctgggcggagggaagggaggtctacagttaccctctggactagtgttgccgggggtacaaggaggtgctttgactgatcatcagagagggcttgtagaagcagtcctcgctttgctcgccaatccgtttgctgaagttccagcggccgttaaggccattgtgtccggcgtcggattcatcgcaacgaacccacctaccgcaccgcatatcgaatatcatattcgcgaggcagccccaggggtcacgtatttccaacacgcgattgattacttgagacaagtaggggcctcggttgcagctcgtgctgcattacttggtgattttttccgcaaatcgaaggagaagattggtaaagaattcaaacgtatcgtccaacgcattaaagacttcctacgaaacttggtaccaaggacggagagttacggacgcaaaaagcggcgacaaaggagacgt

SEQ ID NO: 67 - protein, 295 aa, Artificial sequence
  D29gp12/alpha-defensin/PTD3
MSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQIDCYCRIPACIAGERRYGTCIYQGRLWAFCCYARKARRQARR

SEQ ID NO: 68 - DNA, 885 bp, Artificial sequence
  D29gp12/alpha-defensin/PTD3
atgagtaaaccttggctattcacagtgcacggtacgggtcagcctgacccactaggtcctgggcttccagcagatactgcgcgcgatgtgctcgacatttatcgctggcaacccatcggaaattatcctgcagcggcttttccgatgtggccgtccgttgaaaaaggggtagcagagctcatattacagatcgaactgaaattggatgctgacccgtatgcagattttgcgatggcggggtatagccagggagccatcgttgtgggccaagttcttaagcaccatattctaccaccgaccggacgtctccataggttcctgcatcgtttaaaaaaggtcattttttggggcaaccccatgcggcaaaagggtttcgctcactcggatgagtggatccacccagtagctgcacccgacacgttaggtatcctggaggatcgtctggagaacttagagcagtacggatttgaagtcagagactacgctcatgatggtgatatgtatgcgagcattaaggaagacgatcttcatgagtacgaagtggcgataggcagaatagttatgaaagcctctggatttatcggcgggagggatagtgtcgtagcccaattgattgaattgggccaacggcccataactgagggtattgccctagctggagctataatcgacgcattaaccttctttgcccgatcacggatgggcgacaaatggccacacctctataatagataccctgcggtagaattccttcgtcagattgactgttactgccgcataccggcgtgcatagcaggggaaaggcgctacgggacatgcatatatcagggacgattgtgggccttctgttgttacgccaggaaggcacgaagacaagctcgacgg

SEQ ID NO: 69 - protein, 295 aa, Artificial sequence
  PTD3/alpha-defensin/D29gp12
YARKARRQARRDCYCRIPACIAGERRYGTCIYQGRLWAFCCMSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 70 - DNA, 885 bp, Artificial sequence
  PTD3/alpha-defensin/D29gp12
tacgcacggaaggctcgcagacaggcccgcagagactgttattgtcgtataccagcgtgtatcgctggggagaggcgttatggcacgtgcatctaccaagggaggctgtgggcgttttgctgcatgagcaaaccatggctctttacggtacacggtactggacaaccagacccgctaggcccaggactacccgcggatacagccagagatgtattagatatttatcggtggcaaccgataggaaactaccccgctgcagctttcccaatgtggcctagtgttgaaaaaggtgtcgccgaactgattcttcaaattgaactcaaattagacgcggacccttatgctgattttgcgatggcaggatattcacagggcgcaatagtcgtgggtcaagtgttgaagcatcacatcctgcccccgaccggtcggttacacagatttttgcacaggctaaagaaagttattttctggggtaatcctatgcgccaaaaagggtttgcgcattccgacgagtggatacatccggttgctgcccctgacactcttggcatccttgaggatcgattggagaacttggaacagtacgggttcgaagtacgagattacgcccatgatggcgatatgtacgcaagcattaaggaggatgacctccacgagtatgaggtcgcaattggaaggatcgtgatgaaagcaagtggattcataggtggacgcgactcggtggttgcccagttaattgaactcggtcagcgaccgataacagaaggcatcgctctggccggggctataatcgacgcattaacctttttcgcgcggtctcgtatgggggataagtggccccatctttataatcgataccctgcggtagaattcctacgtcagata

SEQ ID NO: 71 - protein, 325 aa, Artificial sequence
  PTD3/alpha-defensin/D29gp12/alpha-defensin
YARKARRQARRDCYCRIPACIAGERRYGTCIYQGRLWAFCCMSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRIDCYCRIPACIAGERRYGTCIYQGRLWAFCC

SEQ ID NO: 72 - DNA, 975 bp, Artificial sequence
  PTD3/alpha-defensin/D29gp12/alpha-defensin
tacgcccgtaaggcccggagacaggccaggcgtgactgttattgcagaatccccgcatgcattgctggcgagcgtcgatatggtacgtgtatataccaaggccgcctttgggccttttgctgtatgtcaaaaccctggttattcaccgtacacggaacaggtcagcctgatccactgggtccggggcttcctgccgacacagcgcgtgacgtactcgatatctacagatggcagccaatagggaattaccccgcggctgcctttccaatgtggccgtcagtcgagaagggtgttgcagagcttattttgcagattgagttaaagctggacgctgatccatatgctgactttgcactcgctggatatagtcaaggagcaatcgtcgtaggacaggttctgaaacatcacattatcaacccacgaggacggttgcatcgctttttgcataggctccggaaagtaatattttggggtaatcctatgaggcagaaggggtttgcacacacggatgaatggattcaccaagttgcggcaagtgacaccatgggcatactagaggacagattggaaaacttagaacaatacgggttcgaggtgagggattatgcgcacgatggcgatatgtacgcgagcattaaagaagacgatatgcatgaatatgaggtcgcgattgggcgcattgtgatgtctgcgagacgatttataggtggcaaggattcggttatagcccaactgatagaactaggtcaacgacctatctgggaaggaatcgctatggcgcgtgctatcattgacgcactaactttcttcgctaaaagcactcagggaccctcctggccgcatctttataatcggttcccggccgtggaattcctacggcgcatagattgctattgcaggatccctgcatgtatcgcaggggagcgacgctacgggacatgtatataccaaggcagattatgggctttctgctgt

SEQ ID NO: 73 - protein, 303 aa, Artificial sequence
  beta-defensin/D29gp12/TAT47-57
NPVSCVRNKGICVPIRCPGSMKQIGTCVGRAVKCCRKKMSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQIYGRKKRRQRRR

SEQ ID NO: 74 - DNA, 909 bp, Artificial sequence
  beta defensin/D29gp12/TAT47-57
aatcctgtaagttgcgtacggaacaaaggtatatgtgttccaattagatgcccaggtagcatgaaacagatagggacctgcgttggacgggccgtaaaatgttgtcgcaagaaaatgtctaagccgtggctattcacggttcacggtacaggccaacctgacccactcggcccaggacttcctgccgatactgctcgcgacgtgttagatatatatcgttggcagccaattggtaattacccggctgcagctttccccatgtggccctcggtcgagaagggtgtggccgagctcatattgcagattgaattgaaattggatgccgatccgtatgccgactttgctatggcggggtacagtcaaggcgcgatagtcgtaggccaagtgctaaagcatcacatcctccctcccactggacgactccaccgttttctacatcgacttaaaaaggtcatcttttggggaaaccccatgaggcaaaagggctttgcacactccgacgagtggatccatccggttgctgcaccagacacgttgggtattcttgaagacaggctggagaacctggaacagtatgggttcgaagttcgggattatgcgcatgatggggacatgtacgcatcaataaaagaagatgacttacacgaatatgaagtggcaattggaagaatcgtcatgaaggcgtcaggatttattggaggtagggatagcgtggtagcacaactaatcgagctgggccagcgtcccatcaccgaggggatagcgcttgctggggctattatcgacgccctgacattctttgcgcgctctcgaatgggggataaatggcctcatttatacaatcgctatccggcagtcgagttcttaagacagatatacggcagaaagaaaaggagacaacgacggcgt

SEQ ID NO: 75 - protein, 332 aa, Artificial sequence
  beta-defensin/Hepcidin/L5gp12/TAT47-57
NPVSCVRNKGICVPIRCPGSMKQIGTCVGRAVKCCRKKGAGADTHFPICIFCCGCCHRSKCGMCCKTMSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRIYGRKKRRQRRR

SEQ ID NO: 76 - DNA, 996 bp, Artificial sequence
  beta-defensin/Hepcidin/L5gp12/TAT47-57
aatcccgtgagctgtgtaagaaataaaggtatttgtgtgccaattcgatgcccaggaagtatgaagcagattggaacatgtgttggccgtgcggtaaagtgctgtaggaagaaaggcgcaggagctgatactcacttcccaatatgcattttctgctgtgggtgctgtcatcgctcaaaatgcggtatgtgttgcaaaacaatgagtaaaccttggttgttcactgtccatgggaccggccaacctgatcctttggggccgggtttgccagcggatacagctagggatgtcttagatatctaccgttggcaaccgataggcaattaccccgcggctgcgttccctatgtggccgagcgttgaaaagggagtagcggagctgatcctacagattgaactcaagctggacgctgacccctatgctgactttgcacttgcagggtactcgcaaggagccattgtggtcggacaagttctaaaacaccatatcataaacccccgaggccgattacatagattcctacacaggctaaggaaggtaatattttggggtaatcctatgcggcagaagggctttgctcacactgacgagtggatacaccaggtcgcagcttcggataccatgggcatattagaggatcgccttgagaacttggaacaatatggttttgaagttcgggactatgcccacgacggtgacatgtacgcctccatcaaggaggatgacatgcatgagtatgaagtggcgatcgggcgaattgtaatgtcagcccgcagattcatcggagggaaggattctgtgatcgcacaactcatagaactgggacaacggccgatatgggagggtattgccatggcacgggcaattatagacgcgcttacgtttttcgccaaatctacgcagggtccatcctggccgcatctctacaaccgttttcccgccgttgaatttttaaggagaatctatgggcgcaaaaaacgtagacagcgtcgacgg

SEQ ID NO: 77 - protein, 602 aa, Artificial sequence
  TM4gp29/LL-37/TATA47-57
SFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAAGSGSLLGDFFRKSKEKIGKEFKRIVQRIKDFLRNLVPRTESGAGAYGRKKRRQRRR

SEQ ID NO: 78 - DNA, 1806 bp, Artificial sequence
  TM4gp29/LL-37/TAT47-57
agtttcacacgttttttacaagatgacccactattaacacgtgagcaagttatggcggagttaatacgggttgctgacgaacttaatatgccagacaagcgtggagcgtgcgtaatagctggaatgacaattagccaagaggttggtgttaaagacaatgatccaccatttgagaggcgtttctggtgcccagctaatcgtgccgatccagaaagtttcaattatccacacgatagcgaaagcaatgatggtaggagcgttggatattttcaacaacaaaagggtccaaatggagaattgtggtggggtacaacagctagcgaaatgaatttacacagtgcagcgacacaattcatgacaaggctaaaggcggctggatacaatgccagtaatgcgcaagccgctaatgatagcgcacaagccatacaacgaagcggtgttccacaagcatataagcaatggtgggatgacataaatcggctttatgacaaagtaaagggaagcggtggaggtccagcgccagccccaaaaccaccacaaagcggtccatggacaggagatccagtatggttagccgatgtactaagggcagaaggattgaatgttgtagaattaccaggttggctagaccgaggacatggtgatatgggacggttgtggggtgttgtatgtcatcacacaggaagcgataatacaccaagcagtgaaatcgcctttcatccaagccttggattatgcagccaaatacatttggccagaaatggaacagttacattgtgtggagttggtattgcatggcacgcaggtgttggtagttatccaggtctaccagaggacaatgcaaatgctgttacaattggtattgaagctcaaaatagcggaacatatgacggtgcgccacatcggacaaattggccagacgcgcaatacgatgcttacgttaaatgctgtgcagctatctgtcggcgattgggagtaagggcggaccatgtaatcagtcacaaagaatgggcaggtaggaaacaaggtaaatgggacccaggtgcaatagatatgaatatatttagagccgatgtacaacgtcggatcgatgcccatcaaccaaatggagaagacgattttatggcggctttgagcgcagacgaacaacgagaggtattgaatcttttgcgtgtactagccgatcggagattcgtaagtaggagtccattcaggcaccttggagaaggtccaagcgaaacagtagcaggatttggtcttaatacagacggattgaatcatgcccaatatacaattgaacttgcccgattaggtgacccaacacacttggcacttttaagagaggtagcgagtgcggaaggtgacagcagatacccagatcggcaatacgacgccaagcttgcgaaaagggttctagcagaaattgaaggagcagccacagcgccagctaagccaagtacaccaagtgcgccaacagagccagccccagaagctccaacaccaccagttaaggctgcgtgcgccttaagtgcggctggatgtgttgtagcgggtagtacaagtggtggaggttgcgcactaagtacagatggtacaggaaagtgtgtagttacagccgctacagacggtggagcagctggaagtggaagtcttttaggagacttctttcgtaagagcaaagagaaaatcggaaaagaatttaaacgtatcgtacaacgaattaaggatttcctaagaaatctagtaccacgaacagagagtggtgcaggagcttacggtagaaagaaacggcgacaaagacgaaga

SEQ ID NO: 79 - protein, 594 aa, Artificial sequence
  TM4gp29/LL-37/TAT47-57
SFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAALLGDFFRKSKEKIGKEFKRIVQRIKDFLRNLVPRTESYGRKKRRQRRR

SEQ ID NO: 80 - DNA, 1782 bp, Artificial sequence
  TM4gp29/LL-37/TAT47-57
agttttacaaggttcctacaagatgacccactattgacacgagaacaagtaatggctgagctaatacgagtagcagatgaattaaatatgccagataagaggggagcttgtgttatcgccggtatgacaatcagtcaagaagttggagttaaggacaatgatccaccattcgagcgtaggttctggtgtccagcgaatcgtgctgatccagaaagttttaattacccacacgacagcgaaagcaatgacggacgtagtgtaggttatttccaacaacaaaagggaccaaatggagagctttggtggggtacaacagctagtgagatgaatctacatagtgcagcgacacaatttatgacaagacttaaagctgccggttataatgccagtaatgcgcaagcggctaatgacagtgcacaagctatacaacgtagtggtgtaccacaagcctataagcaatggtgggatgacataaataggttatacgataaggtaaagggtagtggtggaggtccagcaccagcaccaaaaccaccacaaagcggtccatggacaggagatccagtttggttggcggacgtattacgagcagaaggtttgaatgtagttgaacttccaggatggcttgacagaggacacggtgacatgggacgtctttggggtgtagtttgtcaccatacaggtagtgacaatacaccaagtagcgaaattgcctttcacccaagtttgggactttgcagccaaattcatcttgccaggaatggtacagttacactatgcggagtaggaatagcttggcacgccggtgttggtagctatccaggtttaccagaagataatgctaatgcagtaacaattggaattgaagcacaaaatagcggaacatacgacggtgcaccacatagaacaaattggccagacgctcaatacgatgcgtatgttaaatgctgtgctgccatatgccggcgattaggtgtacgggctgaccatgttattagccacaaggaatgggccggaagaaaacaaggaaaatgggacccaggagctatcgatatgaatatctttcgggcggatgtacaacggcgaattgatgctcatcaaccaaatggagaagatgactttatggcggccttaagtgccgacgaacaacgtgaggttttaaatcttttacgggtattggcggaccggcgattcgttagtaggagcccattccgacatttgggtgagggaccaagcgagacagtagcgggattcggtttgaatacagatggtttgaatcatgcacaatatacaatagaattggcaagattgggtgacccaacacacctagcgcttttaagggaagtagcaagtgcagaaggtgatagccgttatccagatcgtcaatacgatgccaagctagcgaaacgagttttagcagaaattgaaggagccgcgacagcgccagccaaaccaagcacaccaagtgctccaacagaaccagcaccagaggcgccaacaccaccagttaaagctgcctgtgccctaagtgcagcgggatgcgtagttgccggaagcacaagcggaggtggatgcgctttgagcacagacggaacaggtaaatgtgtagttacagctgcgacagatggtggagcagcattactaggtgactttttcaggaaaagcaaagaaaagatcggtaaagagtttaaaagaatagtacaacggatcaaggattttcttagaaatctagttccacggacagagagctacggaagaaagaagcggagacaaaggcgacgt

SEQ ID NO: 81 - protein, 554 aa, Artificial sequence
  Bzx2gp11/alpha-defensin/PTD3
TEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGENDCYCRIPACIAGERRYGTCIYQGRLWAFCCYARKARRQARR

SEQ ID NO: 82 - DNA, 1662 bp, Artificial sequence
  Bzx2gp11/alpha-defensin/PTD3
acagagaaagttttaccatacgatcggagtatagttacacaagagacaggttggtggtgtggtccagccgcaacacaagtagttttaaatagtcgtggaataatcgtaccagaagctacattggccgctgaaatcgaagccatagaaaatccaggtcgtggtgatgaccgagacggaacagattatgtaggtcttattgagcaagttcttgatcggagggttccacaagctcgttacacaagcgtatatttaacaaatgatccaccaacacaagcacaaaaggaccgattgtgggaacatattgtaagaagcataaatgcaggttacggagtagttatgaattgggtagctccaccaagtaataagccacgtggagttaaaggtagcgttagcccacgatatagtggaggtacaacatatcactatgtagcgtgcatgggatatgacgatacaccaggtgccagagcagtatggatagcggacagcggattccaaccacaaggatactggatcagctttgaccaatgcgccacacttatcccaccaaaaggttatgcatacgcagacgcggcaccagctgcaccagctccagctccaacaccagttgtagacgccgcgccaatattggcccgggcggcaggtattagcgaagcaaaggcaagggaaattcttccaacaatgcgggatggactaaaacaagcggattgtacaacagttaatcgaattgcaatgtttatagcccaaacaggacacgaaagtgatgactttcgggctacagaagagtatgcaaatggaccattggatcaagaacgttggatttacaaaggtcgaacatggatacaaattacatggagggaacactacgctcgtttcggtaaatggtgtttcgaccgtggacttgttacagatccagatgtatttgttaaaaatccacgagctttggccgacctaaaatgggcgggtataggagcggcttggtactggacagtagaacggccagatatcaatgccttatgcgatcgaagagacatcgaaacagttagtagaaggataaatggaacaaatccaaatacaggtagagcgaatcatatcgaggaacgaattgcgaggtggaatcgtgcactagcagtaggtgacgatttacttcaactaattagagaagaggaagacggttttttaagtgctttgacaccagccgagcaacgagcgttgtataatgaaatcatgaagaaaggaccaacacgtagtttcatggctgaggatcaaaatcaaatcgagacattattgggattcgtatacaatatagacggaaatatctggaatgacgctgtaacacgggcttatttatttgatgttccattagccgtagaatacgttgagagggttgcgagggatggagtacatccaaagagctgggcatttcaacaactagacggaaagggagaaaggtggctagccaagttcggacaagaatactgtaaaggtctaatccggttcaagaaaaagttgaatgatctacttgaaccatatggagagaatgactgctactgtcggattccagcgtgcattgctggtgaaaggagatatggtacatgtatatatcaaggtagactttgggccttttgttgctacgcgagaaaggccagaaggcaagcgcgtcga

SEQ ID NO: 83 - protein, 455 aa, Artificial sequence
  TM4gp30/LL-37/TAT47-57
AWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAAGSGSLLGDFFRKSKEKIGKEFKRIVQRIKDFLRNLVPRTESGAGAYGRKKRRQRRR

SEQ ID NO: 84 - DNA, 1365 bp, Artificial sequence
  TM4gp30/LL-37/TAT47-57
gcttgggtcggctggcagcttggtatgcagggtgaacaggttaaggtaatccagcaaaagttgatagccaagtaccagtgggtgcgtgatagatatccccggttgaccgcaagtggggtttacgacgtcaacacccaagcagcgattgttgagtttcaatttagagccggacttccagtcacgggcatagccgactatgcgacacaggtgcggcttggagctgtcgcaccagctccaccccctaggcaacggattatggtactcacatttagcgggacgtcggctgatatgtggacaggctacccggctgacgttgcaagagccctagatccttcaattttttattggcaacccgtttgttatggcccaaacggtattcccgcgatattcccgatggggtcgagtgccaagtccggggaagttgaaggcttgcgactattagacgagaaagctcgcgatttcgactacattgtcctgatcgggtatagtcagggtgcgttgcctgcgagcaggttgatgcgtcgcatcctatcaggtgatctgcaaaggttcaaatcaaaactgatagcaggagtgacgtttgggaacccgatgcgagaaaaaggtcacacctttcctggcggggctgacccgggtggccacggcttagacccacagtgcctcgtgaatactccggattggtggcacgactacgcggccaaaggcgatatctacacagtaggttctggatctaatgatgagaaggccaatgcggacatgactttcatatatcaattagtccaaggagacatactcggtatgatgttcggtacgggtaaccctttggatatattagggttgctgggaggcctaggcggaggtctacttggaggcctaggtgggggcttgcttggaggtggaaaggggggattacaactgcctagcggacttgttttacctggggttcaggggggtgcattaaccgaccatcaacgtggacttgtagaagcggtgctggctttactcgcaaatcccttcgcggaagtacccgccgcggtgaaggcaatcgtaagtggcgtcggtttcatcgccactaatccgccaactgccccacatattgagtaccatattagggaggcagctcccggcgtgacatatttccaacatgcaatcgattatctcaggcaagtcggagcctccgtggctgcacgggctgcgggatctggatcactactgggagattttttccgaaagtctaaagagaaaatagggaaagaatttaagcgaattgtacagcgcatcaaggattttctacgaaacctcgtaccgcgtactgagagcggggcaggggcttacggtcgcaaaaaacgccggcagagacgtaga

SEQ ID NO: 85 - protein, 447 aa, Artificial sequence
  TM4gp30/LL-37/TAT47-57
AWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAALLGDFFRKSKEKIGKEFKRIVQRIKDFLRNLVPRTESYGRKKRRQRRR

SEQ ID NO: 86 - DNA, 1341 bp, Artificial sequence
  TM4gp30/LL-37/TAT47-57
gcctgggttggctggcagttaggaatgcagggtgaacaggtgaaggtaatacagcaaaaacttatagccaagtaccagtgggtcagggatagatatccccgcctaaccgcgagtggggtttacgacgtgaatactcaggcagctatcgttgagttccaatttagagctggcctaccagtaactggtatagcagactatgctactcaagtgcggttaggggcggtcgcgcctgctccgccaccccgtcaacgaatcatggtactgacattttcaggcacatcagcggatatgtggactggatatcccgcagacgtcgcgagggctcttgacccctcaatattctattggcagcccgtgtgctacgggcccaacggaattccggccatattccctatggggtcctctgcaaaatctggcgaggttgagggtttgcgtctactggacgaaaaagcccgagactttgattacatagtgctgataggttatagtcaaggagcgctcccggcgagtaggttaatgcggcgaattctgagcggcgatctccaacggtttaaaagcaagcttatcgcaggtgtaacctttgggaatcctatgcgggagaagggacatacatttcctggaggtgccgacccaggtggacacggcttagatcctcaatgtctcgtcaataccccggattggtggcacgattacgccgcgaagggagatatctacacagttggaagcggtagcaatgacgaaaaagctaacgcggacatgacatttatttatcagctggtgcagggggacatcctaggcatgatgttcggaacgggtaaccctctggatatattgggtcttctcgggggcttaggcgggggcttattgggcggtctagggggcggtcttctgggcggagggaagggaggtctacagttaccctctggactagtgttgccgggggtacaaggaggtgctttgactgatcatcagagagggcttgtagaagcagtcctcgctttgctcgccaatccgtttgctgaagttccagcggccgttaaggccattgtgtccggcgtcggattcatcgcaacgaacccacctaccgcaccgcatatcgaatatcatattcgcgaggcagccccaggggtcacgtatttccaacacgcgattgattacttgagacaagtaggggcctcggttgcagctcgtgctgcattacttggtgattttttccgcaaatcgaaggagaagattggtaaagaattcaaacgtatcgtccaacgcattaaagacttcctacgaaacttggtaccaaggacggagagttacggacgcaaaaagcggcgacaaaggagacgt

SEQ ID NO: 87 - protein, 294 aa, Artificial sequence
  D29/alpha-defensin/PTD3
SKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQIDCYCRIPACIAGERRYGTCIYQGRLWAFCCYARKARRQARR

SEQ ID NO: 88 - DNA, 882 bp, Artificial sequence
  D29gp12/alpha-defensin/PTD3
agtaaaccttggctattcacagtgcacggtacgggtcagcctgacccactaggtcctgggcttccagcagatactgcgcgcgatgtgctcgacatttatcgctggcaacccatcggaaattatcctgcagcggcttttccgatgtggccgtccgttgaaaaaggggtagcagagctcatattacagatcgaactgaaattggatgctgacccgtatgcagattttgcgatggcggggtatagccagggagccatcgttgtgggccaagttcttaagcaccatattctaccaccgaccggacgtctccataggttcctgcatcgtttaaaaaaggtcattttttggggcaaccccatgcggcaaaagggtttcgctcactcggatgagtggatccacccagtagctgcacccgacacgttaggtatcctggaggatcgtctggagaacttagagcagtacggatttgaagtcagagactacgctcatgatggtgatatgtatgcgagcattaaggaagacgatcttcatgagtacgaagtggcgataggcagaatagttatgaaagcctctggatttatcggcgggagggatagtgtcgtagcccaattgattgaattgggccaacggcccataactgagggtattgccctagctggagctataatcgacgcattaaccttctttgcccgatcacggatgggcgacaaatggccacacctctataatagataccctgcggtagaattccttcgtcagattgactgttactgccgcataccggcgtgcatagcaggggaaaggcgctacgggacatgcatatatcagggacgattgtgggccttctgttgttacgccaggaaggcacgaagacaagctcgacgg

SEQ ID NO: 89 - protein, 602 aa, Artificial sequence
  TM4gp29/LL37/TAT47-57 construct 1
SFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAAHHHHHHGSLLGDFFRKSKEKIGKEFKRIVQRIKDFLRNLVPRTESYGRKKRRQRRR

SEQ ID NO: 90 - DNA, 1806 bp, Artificial sequence
  TM4gp29/LL37/TAT47-57 construct 1
agcttcactcgctttctgcaggatgatccgctgctgacccgtgaacaggtgatggctgaactgatccgtgtggcggacgaactgaacatgccggataaacgtggcgcttgcgttatcgccggcatgactatctcccaggaagtgggtgtcaaagacaacgacccaccattcgagcgccgtttttggtgcccggcaaaccgtgctgatccggaaagcttcaactacccgcacgactctgaatccaacgacggtcgctccgtaggctacttccagcagcagaaaggcccgaacggtgaactgtggtggggtacgaccgcttccgagatgaacctgcattctgccgcaactcagttcatgacgcgtctgaaagccgcaggctacaacgcaagcaacgcacaggctgcgaacgactccgcacaggcgatccagcgtagcggcgtgccgcaggcctacaaacagtggtgggacgacattaaccgcctgtatgacaaagtgaaaggctctggtggcggtccggcaccggcaccaaaaccaccacagagcggtccgtggactggtgatccggtttggctggctgacgttctccgcgctgagggtctgaacgtagtggaactgccaggttggctggatcgtggccacggtgacatgggtcgtctgtggggtgtcgtgtgccatcatactggctccgataacacgccaagctccgaaatcgctttccacccgagcctgggtctgtgttctcagatccacctggctcgtaacggtaccgttaccctgtgcggtgtaggtatcgcttggcacgcaggtgttggttcctacccgggtctcccggaagacaacgcgaacgcggtcactattggcatcgaagctcagaactctggtacgtacgacggcgctccacaccgtacgaactggccagatgcgcagtatgacgcgtatgtaaaatgctgtgccgccatctgtcgtcgcctgggcgtacgcgctgatcacgttatttcccacaaagaatgggctggtcgtaaacagggcaaatgggacccgggcgctatcgatatgaacatcttccgtgctgacgtccagcgtcgtattgacgcacaccagccgaacggtgaggacgactttatggcagcgctgtctgcggatgagcagcgtgaagtgctgaacctgctgcgtgtcctggcagatcgccgttttgtatctcgctctccgttccgtcacctgggtgaaggtccaagcgagacggttgcaggtttcggcctgaacaccgacggcctgaaccatgcgcagtatactatcgaactggcacgtctgggcgatccaacccacctggctctgctgcgcgaagttgcctctgcagaaggcgattctcgctacccagatcgccagtacgatgcgaaactggccaaacgtgtcctggcggaaattgaaggtgcagcgaccgctccagctaaaccgtctaccccaagcgctccaactgaaccggctccagaagccccgactccgccagtaaaagctgcctgtgcgctgtccgctgccggttgtgttgtcgctggctctacctctggtggcggttgcgcactgtctactgatggcaccggcaaatgcgtggttactgcggcaactgacggcggtgctgcacaccatcaccatcaccatggatccttattgggtgatttctttcggaagagcaaagaaaagataggaaaggagtttaaacgaattgttcaacgtatcaaagacttcctaaggaatcttgtaccaagaacagaaagttatggccgcaaaaaacggcgtcagcgtcgccgc

SEQ ID NO: 91 - protein, 562 aa, Artificial sequence
  Bxz2gp11/alpha-defensin/PTD3 construct 2
TEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGENHHHHHHGSDCYCRIPACIAGERRYGTCIYQGRLWAFCCYARKARRQARR

SEQ ID NO: 92 - DNA, 1686 bp, Artificial sequence
  Bxz2gp11/alpha-defensin/PTD3 construct 2
accgagaaagtgctgccgtacgaccgttctatcgttacccaggaaaccggttggtggtgtggtccagcggctactcaggttgttctgaactcccgtggcatcatcgttccagaagctacgctggcagcggaaatcgaagctattgaaaacccgggtcgtggtgacgatcgtgatggtaccgactacgtaggtctgatcgagcaggttctggatcgtcgtgttccgcaggctcgttacacctccgtgtatctgaccaacgatccaccgactcaggcacagaaagaccgtctctgggagcatatcgtccgttctatcaacgccggttatggcgtggttatgaactgggtagccccaccgagcaacaaaccacgtggcgtgaaaggctctgtgtctccgcgctattccggcggtaccacttaccactacgtagcctgtatgggttacgacgatacgccaggcgctcgtgcggtttggatcgcggattctggtttccagccacagggctactggattagcttcgatcagtgcgcgaccctgattccgccaaaaggctacgcttatgcagacgcagctccggctgcaccagcaccagctccaactccggttgtagacgctgcaccaattctggctcgtgcggcaggtatctccgaagccaaagcgcgtgaaattctgccgactatgcgcgacggtctgaaacaggctgattgtacgaccgtcaaccgtatcgcaatgtttattgcgcagaccggtcacgaatctgatgacttccgcgccaccgaagagtatgcgaacggtccactggaccaggaacgttggatctacaaaggccgtacctggattcagatcacctggcgtgaacactacgctcgtttcggcaaatggtgcttcgatcgcggcctggtaactgatccggatgttttcgtgaaaaacccacgcgctctggcagatctgaaatgggctggtattggcgcagcgtggtattggaccgttgaacgtccggacatcaacgcactgtgcgaccgccgtgatatcgaaactgtgtctcgtcgcatcaacggcactaacccgaacactggccgcgcgaaccacatcgaggaacgtattgctcgctggaaccgtgcactggctgtgggtgatgacctgctccagctgatccgtgaagaggaagatggtttcctgagcgctctgaccccagcagaacagcgtgccctgtacaacgagattatgaaaaaaggcccaacccgctcttttatggccgaagaccagaaccagatcgagaccctgctgggttttgtctataacatcgacggcaacatctggaacgacgcagttactcgcgcgtatctgttcgacgtaccactggccgtcgaatacgtggaacgcgttgctcgtgatggtgtacacccgaaaagctgggcgtttcagcagctggacggtaaaggcgaacgttggctcgcgaaattcggtcaggaatactgcaaaggtctgatccgcttcaaaaaaaaactgaacgacctgctggaaccgtacggtgaaaaccaccatcaccatcaccacggatccgactgttactgccgtatcccagcatgtattgctggtgaacgccgttacggcacttgtatttaccagggtcgcctgtgggctttttgctgctatgcccgtaaagcacgtcggcaggcgcgccgc

SEQ ID NO: 93 - protein, 557 aa, Artificial sequence
  PTD3/alpha-defensin/Bxz2gp11 construct 3
YARKARRQARRDCYCRIPACIAGERRYGTCIYQGRLWAFCCLKMTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGEN

SEQ ID NO: 94 - DNA, 1671 bp, Artificial sequence
  PTD3/alpha-defensin/Bxz2gp11 construct 3
tatgcccgtaaagcacgtcggcaggcgcgccgcgactgttactgccgtatcccagcatgtattgctggtgaacgccgttacggcacttgtatttaccagggtcgcctgtgggctttttgctgccttaagatgaccgagaaagtgctgccgtacgaccgttctatcgttacccaggaaaccggttggtggtgtggtccagcggctactcaggttgttctgaactcccgtggcatcatcgttccagaagctacgctggcagcggaaatcgaagctattgaaaacccgggtcgtggtgacgatcgtgatggtaccgactacgtaggtctgatcgagcaggttctggatcgtcgtgttccgcaggctcgttacacctccgtgtatctgaccaacgatccaccgactcaggcacagaaagaccgtctctgggagcatatcgtccgttctatcaacgccggttatggcgtggttatgaactgggtagccccaccgagcaacaaaccacgtggcgtgaaaggctctgtgtctccgcgctattccggcggtaccacttaccactacgtagcctgtatgggttacgacgatacgccaggcgctcgtgcggtttggatcgcggattctggtttccagccacagggctactggattagcttcgatcagtgcgcgaccctgattccgccaaaaggctacgcttatgcagacgcagctccggctgcaccagcaccagctccaactccggttgtagacgctgcaccaattctggctcgtgcggcaggtatctccgaagccaaagcgcgtgaaattctgccgactatgcgcgacggtctgaaacaggctgattgtacgaccgtcaaccgtatcgcaatgtttattgcgcagaccggtcacgaatctgatgacttccgcgccaccgaagagtatgcgaacggtccactggaccaggaacgttggatctacaaaggccgtacctggattcagatcacctggcgtgaacactacgctcgtttcggcaaatggtgcttcgatcgcggcctggtaactgatccggatgttttcgtgaaaaacccacgcgctctggcagatctgaaatgggctggtattggcgcagcgtggtattggaccgttgaacgtccggacatcaacgcactgtgcgaccgccgtgatatcgaaactgtgtctcgtcgcatcaacggcactaacccgaacactggccgcgcgaaccacatcgaggaacgtattgctcgctggaaccgtgcactggctgtgggtgatgacctgctccagctgatccgtgaagaggaagatggtttcctgagcgctctgaccccagcagaacagcgtgccctgtacaacgagattatgaaaaaaggcccaacccgctcttttatggccgaagaccagaaccagatcgagaccctgctgggttttgtctataacatcgacggcaacatctggaacgacgcagttactcgcgcgtatctgttcgacgtaccactggccgtcgaatacgtggaacgcgttgctcgtgatggtgtacacccgaaaagctgggcgtttcagcagctggacggtaaaggcgaacgttggctcgcgaaattcggtcaggaatactgcaaaggtctgatccgcttcaaaaaaaaactgaacgacctgctggaaccgtacggtgaaaac

SEQ ID NO: 95 - protein, 595 aa, Artificial sequence
  PTD3/alpha-defensin/Bxz2gp11/alpha-defensin construct 4
YARKARRQARRDCYCRIPACIAGERRYGTCIYQGRLWAFCCLKMTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGENHHHHHHGSDCYCRIPACIAGERRYGTCIYQGRLWAFCC

SEQ ID NO: 96 - DNA, 1785 bp, Artificial sequence
  PTD3/alpha-defensin/Bxz2gp11/alpha-defensin construct 4
tatgcccgtaaagcacgtcggcaggcgcgccgcgactgttactgccgtatcccagcatgtattgctggtgaacgccgttacggcacttgtatttaccagggtcgcctgtgggctttttgctgccttaagatgaccgagaaagtgctgccgtacgaccgttctatcgttacccaggaaaccggttggtggtgtggtccagcggctactcaggttgttctgaactcccgtggcatcatcgttccagaagctacgctggcagcggaaatcgaagctattgaaaacccgggtcgtggtgacgatcgtgatggtaccgactacgtaggtctgatcgagcaggttctggatcgtcgtgttccgcaggctcgttacacctccgtgtatctgaccaacgatccaccgactcaggcacagaaagaccgtctctgggagcatatcgtccgttctatcaacgccggttatggcgtggttatgaactgggtagccccaccgagcaacaaaccacgtggcgtgaaaggctctgtgtctccgcgctattccggcggtaccacttaccactacgtagcctgtatgggttacgacgatacgccaggcgctcgtgcggtttggatcgcggattctggtttccagccacagggctactggattagcttcgatcagtgcgcgaccctgattccgccaaaaggctacgcttatgcagacgcagctccggctgcaccagcaccagctccaactccggttgtagacgctgcaccaattctggctcgtgcggcaggtatctccgaagccaaagcgcgtgaaattctgccgactatgcgcgacggtctgaaacaggctgattgtacgaccgtcaaccgtatcgcaatgtttattgcgcagaccggtcacgaatctgatgacttccgcgccaccgaagagtatgcgaacggtccactggaccaggaacgttggatctacaaaggccgtacctggattcagatcacctggcgtgaacactacgctcgtttcggcaaatggtgcttcgatcgcggcctggtaactgatccggatgttttcgtgaaaaacccacgcgctctggcagatctgaaatgggctggtattggcgcagcgtggtattggaccgttgaacgtccggacatcaacgcactgtgcgaccgccgtgatatcgaaactgtgtctcgtcgcatcaacggcactaacccgaacactggccgcgcgaaccacatcgaggaacgtattgctcgctggaaccgtgcactggctgtgggtgatgacctgctccagctgatccgtgaagaggaagatggtttcctgagcgctctgaccccagcagaacagcgtgccctgtacaacgagattatgaaaaaaggcccaacccgctcttttatggccgaagaccagaaccagatcgagaccctgctgggttttgtctataacatcgacggcaacatctggaacgacgcagttactcgcgcgtatctgttcgacgtaccactggccgtcgaatacgtggaacgcgttgctcgtgatggtgtacacccgaaaagctgggcgtttcagcagctggacggtaaaggcgaacgttggctcgcgaaattcggtcaggaatactgcaaaggtctgatccgcttcaaaaaaaaactgaacgacctgctggaaccgtacggtgaaaaccaccatcaccatcaccacggatccgactgttactgccgtatcccagcatgtattgctggtgaacgccgttacggcacttgtatttaccagggtcgcctgtgggctttttgctgc

SEQ ID NO: 97 - protein, 351 aa, Artificial sequence
  beta-defensin/L5gp10/TAT47-57 construct 5
NPVSCVRNKGICVPIRCPGSMKQIGTCVGRAVKCCRKKLKMTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGLHHHHHHGSYGRKKRRQRRR

SEQ ID NO: 98 - DNA, 1053 bp, Artificial sequence
  beta-defensin/L5gp10/TAT47-57 construct 5
aacccggtatcttgcgtgcgcaacaaaggtatctgcgttccgattcgttgtccgggttctatgaaacagattggcacttgcgttggtcgtgccgtgaaatgctgccgtaaaaaacttaagatgaccttcactgttacccgtgaacgtgcgcagtgggttcatgatatggcacgtgctcgcgatggcctgccgtatgcgtatggcggtgcgttcaccaacaacccgcgtgtaagcaccgattgctctggtctggtactgcagactggcgcttggtatggtggtcgtaccgattgggtaggcaaccgttacggctccaccgaatcttttcgcctggaccacaaaatcgtctacgacctgggcttcaaacgtatgccacgtggtggtccagccgcactgccgatcaaaccggtgatgctggttggtctgcagcatggcggtggtggcgtttacagccacacggcttgcaccctgatgactatggatcacccaggtggcccagttaaaatgtccgaccgtggcgttgactgggaatctcatggcaaccgcaacggtgtaggtgttgaactgtacgaaggtgcgcgcgcttggaacgatccgctcttccacgacttttggtacctggatgccgttctggaagacgaaggcgacgatgacgaactggcagatccggtgctgggcaaaatgattcgcgagatccacgcgtgtctgttcaaccagacggcgagcacttctgatctggcaaccccaggtgaaggtgcgatctggcagctgcaccagaaaatccactctatcgacggtatgctgcacccgattcatgctgaacgccgtgcacgtgcaggtgacctgggcgaactccaccgtatcgtactggctgccaaaggcctgggtgtgaaacgcgacgaggtgaccaaacgtgtctaccagtctatcctggccgacattgagcgtgacaacccggaagtgctgcagcgttacattgctgagcgtggtggtctgcaccaccatcaccaccacggatcctatggccgcaaaaaacggcgtcagcgtcgccgc

SEQ ID NO: 99 - protein, 368 aa, Artificial sequence
  alpha-defensin/hepcidin/L5gp10/TAT47-57 construct 6
DCYCRIPACIAGERRYGTCIYQGRLWAFCCDTHFPICIFCCGCCHRSKCGMCCKTLKMTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGLHHHHHHGSYGRKKRRQRRR

SEQ ID NO: 100 - DNA, 1104 bp, Artificial sequence
  alpha-defensin/hepcidin/L5gp10/TAT47-57 construct 6
gactgttactgccgtatcccagcatgtattgctggtgaacgccgttacggcacttgtatttaccagggtcgcctgtgggctttttgctgcgacacccacttcccgatttgtatcttctgctgtggctgctgtcaccgctccaaatgtggtatgtgctgtaaaacccttaagatgaccttcactgttacccgtgaacgtgcgcagtgggttcatgatatggcacgtgctcgcgatggcctgccgtatgcgtatggcggtgcgttcaccaacaacccgcgtgtaagcaccgattgctctggtctggtactgcagactggcgcttggtatggtggtcgtaccgattgggtaggcaaccgttacggctccaccgaatcttttcgcctggaccacaaaatcgtctacgacctgggcttcaaacgtatgccacgtggtggtccagccgcactgccgatcaaaccggtgatgctggttggtctgcagcatggcggtggtggcgtttacagccacacggcttgcaccctgatgactatggatcacccaggtggcccagttaaaatgtccgaccgtggcgttgactgggaatctcatggcaaccgcaacggtgtaggtgttgaactgtacgaaggtgcgcgcgcttggaacgatccgctcttccacgacttttggtacctggatgccgttctggaagacgaaggcgacgatgacgaactggcagatccggtgctgggcaaaatgattcgcgagatccacgcgtgtctgttcaaccagacggcgagcacttctgatctggcaaccccaggtgaaggtgcgatctggcagctgcaccagaaaatccactctatcgacggtatgctgcacccgattcatgctgaacgccgtgcacgtgcaggtgacctgggcgaactccaccgtatcgtactggctgccaaaggcctgggtgtgaaacgcgacgaggtgaccaaacgtgtctaccagtctatcctggccgacattgagcgtgacaacccggaagtgctgcagcgttacattgctgagcgtggtggtctgcaccaccatcaccaccacggatcctatggccgcaaaaaacggcgtcagcgtcgccgc

SEQ ID NO: 101 - protein, 455 aa, Artificial sequence
  TM4gp30/LL37/TAT47-57 construct 7
AWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAAHHHHHHGSLLGDFFRKSKEKIGKEFKRIVQRIKDFLRNLVPRTESYGRKKRRQRRR

SEQ ID NO: 102 - DNA, 1365 bp, Artificial sequence
  TM4gp30/LL37/TAT47-57 construct 7
gcttgggtgggttggcagctgggcatgcagggcgaacaggtcaaagtgatccagcagaaactgatcgcgaaataccagtgggtccgtgaccgttatccgcgtctgaccgcttccggtgtttatgatgttaacacgcaggctgcgatcgtggaatttcagttccgtgcaggtctgccggtcactggcattgcggattacgctacccaggttcgtctgggtgccgtagcaccggctccaccaccacgtcagcgtatcatggtcctgacgttttctggtacttctgcggatatgtggaccggttacccagcagacgttgctcgcgcgctggacccgtccattttctactggcagccagtatgctacggtccgaacggtatcccagcgattttcccgatgggtagctccgcgaaatccggtgaagtggaaggcctgcgcctgctggacgagaaagcacgtgacttcgattacatcgtactgattggctacagccagggtgcactgccggcatctcgcctgatgcgccgtattctgtctggcgacctgcagcgcttcaaaagcaaactgatcgctggcgtgaccttcggcaacccgatgcgtgaaaaaggccacaccttcccaggtggcgcagacccaggcggtcatggcctggacccacagtgtctggtgaacaccccagattggtggcacgattacgcggccaaaggcgatatctatactgtgggctccggttctaacgacgaaaaagccaacgccgacatgacctttatctatcagctcgtgcagggtgacattctgggcatgatgtttggcaccggtaacccgctggacatcctgggcctgctcggcggtctgggtggtggtctgctgggtggtctgggtggtggcctgctgggcggtggcaaaggtggtctgcagctgccgtctggtctggtactgccaggtgttcagggcggtgccctgactgatcaccagcgtggtctggtagaagctgttctggcgctgctcgctaacccgttcgcagaagtaccagctgcggtcaaagcgatcgttagcggtgttggcttcatcgctaccaacccgccgactgcaccgcacatcgagtatcacattcgcgaagcagctccaggtgtgacctacttccagcacgcaatcgattacctgcgtcaggttggtgcctctgttgctgcacgtgccgcacaccatcaccatcaccatggatccttattgggtgatttctttcggaagagcaaagaaaagataggaaaggagtttaaacgaattgttcaacgtatcaaagacttcctaaggaatcttgtaccaagaacagaaagttatggccgcaaaaaacggcgtcagcgtcgccgc

SEQ ID NO: 103 - protein, 302 aa, Artificial sequence
  D29gp12/alpha-defensin/PTD3 construct 8
SKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQIHHHHHHGSDCYCRIPACIAGERRYGTCIYQGRLWAFCCYARKARRQARR

SEQ ID NO: 104 - DNA, 906 bp, Artificial sequence
  D20gp12/alpha-defensin/PTD3 construct 8
agcaaaccgtggctgttcaccgtgcatggtactggccagccagatccgctgggtccaggtctgccagctgatactgctcgtgacgtcctggatatctaccgttggcagccgattggtaactacccggctgcagcgttcccaatgtggccgtccgttgaaaaaggcgtagcggaactcatcctgcagatcgaactgaaactggatgcggacccgtatgctgatttcgcgatggctggctactctcagggtgctatcgtagtaggccaggtcctgaaacatcacatcctgccaccgactggtcgtctgcaccgtttcctgcaccgcctgaaaaaagtgatcttctggggtaacccgatgcgtcagaaaggctttgcacactccgacgagtggattcacccagttgctgcaccggatactctgggcatcctggaagatcgcctggagaacctggaacagtacggtttcgaagtgcgtgactatgcccacgacggtgatatgtacgcctccatcaaagaggacgatctgcacgagtacgaggtagcgatcggtcgtatcgttatgaaagcgagcggtttcattggcggccgtgattctgtagtggcgcagctgatcgaactgggtcagcgcccgattacggaaggtattgcactggcaggcgcgattattgacgccctgaccttctttgcccgctctcgtatgggcgacaaatggccacacctgtataaccgctacccagcagttgagtttctgcgtcagatccaccatcaccatcaccatggatccgactgttactgccgtatcccagcatgtattgctggtgaacgccgttacggcacttgtatttaccagggtcgcctgtgggctttttgctgctatgcccgtaaagcacgtcggcaggcgcgccgc

SEQ ID NO: 105 - protein, 297 aa, Artificial sequence
  PTD3/alpha-defensin/D29gp12 construct 9
YARKARRQARRDCYCRIPACIAGERRYGTCIYQGRLWAFCCLKMSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 106 - DNA, 891 bp, Artificial sequence
  PTD3/alpha-defensin/D29gp12 construct 9
tatgcccgtaaagcacgtcggcaggcgcgccgcgactgttactgccgtatcccagcatgtattgctggtgaacgccgttacggcacttgtatttaccagggtcgcctgtgggctttttgctgccttaagatgagcaaaccgtggctgttcaccgtgcatggtactggccagccagatccgctgggtccaggtctgccagctgatactgctcgtgacgtcctggatatctaccgttggcagccgattggtaactacccggctgcagcgttcccaatgtggccgtccgttgaaaaaggcgtagcggaactcatcctgcagatcgaactgaaactggatgcggacccgtatgctgatttcgcgatggctggctactctcagggtgctatcgtagtaggccaggtcctgaaacatcacatcctgccaccgactggtcgtctgcaccgtttcctgcaccgcctgaaaaaagtgatcttctggggtaacccgatgcgtcagaaaggctttgcacactccgacgagtggattcacccagttgctgcaccggatactctgggcatcctggaagatcgcctggagaacctggaacagtacggtttcgaagtgcgtgactatgcccacgacggtgatatgtacgcctccatcaaagaggacgatctgcacgagtacgaggtagcgatcggtcgtatcgttatgaaagcgagcggtttcattggcggccgtgattctgtagtggcgcagctgatcgaactgggtcagcgcccgattacggaaggtattgcactggcaggcgcgattattgacgccctgaccttctttgcccgctctcgtatgggcgacaaatggccacacctgtataaccgctacccagcagttgagtttctgcgtcagatc

SEQ ID NO: 107 - protein, 335 aa, Artificial sequence
  PTD3/alpha-defensin/D29gp12/alpha-defensin construct 10
YARKARRQARRDCYCRIPACIAGERRYGTCIYQGRLWAFCCLKMSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQIHHHHHHGSDCYCRIPACIAGERRYGTCIYQGRLWAFCC

SEQ ID NO: 108 - DNA, 1005 bp, Artificial sequence
  PTD3/alpha-defensin/D29gp12/alpha-defensin construct 10
tatgcccgtaaagcacgtcggcaggcgcgccgcgactgttactgccgtatcccagcatgtattgctggtgaacgccgttacggcacttgtatttaccagggtcgcctgtgggctttttgctgccttaagatgagcaaaccgtggctgttcaccgtgcatggtactggccagccagatccgctgggtccaggtctgccagctgatactgctcgtgacgtcctggatatctaccgttggcagccgattggtaactacccggctgcagcgttcccaatgtggccgtccgttgaaaaaggcgtagcggaactcatcctgcagatcgaactgaaactggatgcggacccgtatgctgatttcgcgatggctggctactctcagggtgctatcgtagtaggccaggtcctgaaacatcacatcctgccaccgactggtcgtctgcaccgtttcctgcaccgcctgaaaaaagtgatcttctggggtaacccgatgcgtcagaaaggctttgcacactccgacgagtggattcacccagttgctgcaccggatactctgggcatcctggaagatcgcctggagaacctggaacagtacggtttcgaagtgcgtgactatgcccacgacggtgatatgtacgcctccatcaaagaggacgatctgcacgagtacgaggtagcgatcggtcgtatcgttatgaaagcgagcggtttcattggcggccgtgattctgtagtggcgcagctgatcgaactgggtcagcgcccgattacggaaggtattgcactggcaggcgcgattattgacgccctgaccttctttgcccgctctcgtatgggcgacaaatggccacacctgtataaccgctacccagcagttgagtttctgcgtcagatccaccatcaccatcaccatggatccgactgttactgccgtatcccagcatgtattgctggtgaacgccgttacggcacttgtatttaccagggtcgcctgtgggctttttgctgc

SEQ ID NO: 109 - protein, 313 aa, Artificial sequence
  beta-defensin/D29gp12/TAT47-57 construct 11
NPVSCVRNKGICVPIRCPGSMKQIGTCVGRAVKCCRKKLKMSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQIHHHHHHGSYGRKKRRQRRR

SEQ ID NO: 110 - DNA, 939 bp, Artificial sequence
  beta-defensin/D29gp12/TAT47-57 construct 11
aacccggtatcttgcgtgcgcaacaaaggtatctgcgttccgattcgttgtccgggttctatgaaacagattggcacttgcgttggtcgtgccgtgaaatgctgccgtaaaaaacttaagatgagcaaaccgtggctgttcaccgtgcatggtactggccagccagatccgctgggtccaggtctgccagctgatactgctcgtgacgtcctggatatctaccgttggcagccgattggtaactacccggctgcagcgttcccaatgtggccgtccgttgaaaaaggcgtagcggaactcatcctgcagatcgaactgaaactggatgcggacccgtatgctgatttcgcgatggctggctactctcagggtgctatcgtagtaggccaggtcctgaaacatcacatcctgccaccgactggtcgtctgcaccgtttcctgcaccgcctgaaaaaagtgatcttctggggtaacccgatgcgtcagaaaggctttgcacactccgacgagtggattcacccagttgctgcaccggatactctgggcatcctggaagatcgcctggagaacctggaacagtacggtttcgaagtgcgtgactatgcccacgacggtgatatgtacgcctccatcaaagaggacgatctgcacgagtacgaggtagcgatcggtcgtatcgttatgaaagcgagcggtttcattggcggccgtgattctgtagtggcgcagctgatcgaactgggtcagcgcccgattacggaaggtattgcactggcaggcgcgattattgacgccctgaccttctttgcccgctctcgtatgggcgacaaatggccacacctgtataaccgctacccagcagttgagtttctgcgtcagatccaccatcaccatcaccatggatcctatggccgcaaaaaacggcgtcagcgtcgccgc

SEQ ID NO: 111 - protein, 338 aa, Artificial sequence
  beta-defensin/hepcidin/L5gp12/TAT47-57 construct 12
NPVSCVRNKGICVPIRCPGSMKQIGTCVGRAVKCCRKKDTHFPICIFCCGCCHRSKCGMCCKTLKMSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRIHHHHHHGSYGRKKRRQRRR

SEQ ID NO: 112 - DNA, 1014 bp, Artificial sequence
  beta-defensin/hepcidin/L5gp12/TAT47-57 construct 12
aacccggtatcttgcgtgcgcaacaaaggtatctgcgttccgattcgttgtccgggttctatgaaacagattggcacttgcgttggtcgtgccgtgaaatgctgccgtaaaaaagacacccacttcccgatttgtatcttctgctgtggctgctgtcaccgctccaaatgtggtatgtgctgtaaaacccttaagatgagcaaaccgtggctgttcaccgttcatggtactggccagccagatccactgggtccaggtctgccagcagatactgcacgtgacgtgctggatatctaccgttggcagccgattggcaactatccagctgcagcgttcccaatgtggccatctgttgagaaaggcgtagccgagctgattctgcagatcgaactgaaactcgacgctgacccgtatgcggatttcgcactggctggttactctcagggtgctatcgtagtaggccaggtcctgaaacaccatatcattaacccgcgtggtcgtctgcaccgttttctgcatcgcctgcgcaaagtgatcttctggggtaacccaatgcgccagaaaggcttcgcacacaccgacgagtggattcaccaggttgcagcgtccgacactatgggcattctggaagatcgcctggaaaacctggaacagtacggcttcgaagtccgtgactatgcccacgatggcgatatgtacgcgtccatcaaagaggacgatatgcacgagtacgaagtagcgatcggtcgtatcgtgatgtctgctcgccgtttcatcggtggcaaagacagcgtgattgcccagctgatcgaactgggtcagcgtccgatctgggaaggtatcgctatggctcgcgctattatcgacgccctcaccttctttgcgaaatctacgcagggtccgtcttggccacacctgtacaaccgtttcccagcagtcgagtttctgcgtcgtatccaccatcaccaccatcacggatcctatggccgcaaaaaacggcgtcagcgtcgccgc

SEQ ID NO: 113 - protein, 731 aa, Artificial sequence [sequence omitted]

SEQ ID NO: 114 - DNA, 2193 bp, Artificial sequence
  TM4gp29/LL37/TAT47-57 construct 1 Thioredoxin tag
agcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctgggtcaggatcaggagcaggtaccgacgacgacgacaaggccatggcaatgagcttcactcgctttctgcaggatgatccgctgctgacccgtgaacaggtgatggctgaactgatccgtgtggcggacgaactgaacatgccggataaacgtggcgcttgcgttatcgccggcatgactatctcccaggaagtgggtgtcaaagacaacgacccaccattcgagcgccgtttttggtgcccggcaaaccgtgctgatccggaaagcttcaactacccgcacgactctgaatccaacgacggtcgctccgtaggctacttccagcagcagaaaggcccgaacggtgaactgtggtggggtacgaccgcttccgagatgaacctgcattctgccgcaactcagttcatgacgcgtctgaaagccgcaggctacaacgcaagcaacgcacaggctgcgaacgactccgcacaggcgatccagcgtagcggcgtgccgcaggcctacaaacagtggtgggacgacattaaccgcctgtatgacaaagtgaaaggctctggtggcggtccggcaccggcaccaaaaccaccacagagcggtccgtggactggtgatccggtttggctggctgacgttctccgcgctgagggtctgaacgtagtggaactgccaggttggctggatcgtggccacggtgacatgggtcgtctgtggggtgtcgtgtgccatcatactggctccgataacacgccaagctccgaaatcgctttccacccgagcctgggtctgtgttctcagatccacctggctcgtaacggtaccgttaccctgtgcggtgtaggtatcgcttggcacgcaggtgttggttcctacccgggtctcccggaagacaacgcgaacgcggtcactattggcatcgaagctcagaactctggtacgtacgacggcgctccacaccgtacgaactggccagatgcgcagtatgacgcgtatgtaaaatgctgtgccgccatctgtcgtcgcctgggcgtacgcgctgatcacgttatttcccacaaagaatgggctggtcgtaaacagggcaaatgggacccgggcgctatcgatatgaacatcttccgtgctgacgtccagcgtcgtattgacgcacaccagccgaacggtgaggacgactttatggcagcgctgtctgcggatgagcagcgtgaagtgctgaacctgctgcgtgtcctggcagatcgccgttttgtatctcgctctccgttccgtcacctgggtgaaggtccaagcgagacggttgcaggtttcggcctgaacaccgacggcctgaaccatgcgcagtatactatcgaactggcacgtctgggcgatccaacccacctggctctgctgcgcgaagttgcctctgcagaaggcgattctcgctacccagatcgccagtacgatgcgaaactggccaaacgtgtcctggcggaaattgaaggtgcagcgaccgctccagctaaaccgtctaccccaagcgctccaactgaaccggctccagaagccccgactccgccagtaaaagctgcctgtgcgctgtccgctgccggttgtgttgtcgctggctctacctctggtggcggttgcgcactgtctactgatggcaccggcaaatgcgtggttactgcggcaactgacggcggtgctgcacaccatcaccatcaccatggatccttattgggtgatttctttcggaagagcaaagaaaagataggaaaggagtttaaacgaattgttcaacgtatcaaagacttcctaaggaatcttgtaccaagaacagaaagttatggccgcaaaaaacggcgtcagcgtcgccgc

SEQ ID NO: 115 - protein, 686 aa, Artificial sequence
  PTD3/alpha-defensin/Bxz2gp11 construct 3 Thioredoxin tag
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSGSGAGTDDDDKAMAMYARKARRQARRDCYCRIPACIAGERRYGTCIYQGRLWAFCCLKMTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGEN

SEQ ID NO: 116 - DNA, 2058 bp, Artificial sequence
  PTD3/alpha-defensin/Bxz2gp11 construct 3 Thioredoxin tag
agcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctgggtcaggatcaggagcaggtaccgacgacgacgacaaggccatggcaatgtatgcccgtaaagcacgtcggcaggcgcgccgcgactgttactgccgtatcccagcatgtattgctggtgaacgccgttacggcacttgtatttaccagggtcgcctgtgggctttttgctgccttaagatgaccgagaaagtgctgccgtacgaccgttctatcgttacccaggaaaccggttggtggtgtggtccagcggctactcaggttgttctgaactcccgtggcatcatcgttccagaagctacgctggcagcggaaatcgaagctattgaaaacccgggtcgtggtgacgatcgtgatggtaccgactacgtaggtctgatcgagcaggttctggatcgtcgtgttccgcaggctcgttacacctccgtgtatctgaccaacgatccaccgactcaggcacagaaagaccgtctctgggagcatatcgtccgttctatcaacgccggttatggcgtggttatgaactgggtagccccaccgagcaacaaaccacgtggcgtgaaaggctctgtgtctccgcgctattccggcggtaccacttaccactacgtagcctgtatgggttacgacgatacgccaggcgctcgtgcggtttggatcgcggattctggtttccagccacagggctactggattagcttcgatcagtgcgcgaccctgattccgccaaaaggctacgcttatgcagacgcagctccggctgcaccagcaccagctccaactccggttgtagacgctgcaccaattctggctcgtgcggcaggtatctccgaagccaaagcgcgtgaaattctgccgactatgcgcgacggtctgaaacaggctgattgtacgaccgtcaaccgtatcgcaatgtttattgcgcagaccggtcacgaatctgatgacttccgcgccaccgaagagtatgcgaacggtccactggaccaggaacgttggatctacaaaggccgtacctggattcagatcacctggcgtgaacactacgctcgtttcggcaaatggtgcttcgatcgcggcctggtaactgatccggatgttttcgtgaaaaacccacgcgctctggcagatctgaaatgggctggtattggcgcagcgtggtattggaccgttgaacgtccggacatcaacgcactgtgcgaccgccgtgatatcgaaactgtgtctcgtcgcatcaacggcactaacccgaacactggccgcgcgaaccacatcgaggaacgtattgctcgctggaaccgtgcactggctgtgggtgatgacctgctccagctgatccgtgaagaggaagatggtttcctgagcgctctgaccccagcagaacagcgtgccctgtacaacgagattatgaaaaaaggcccaacccgctcttttatggccgaagaccagaaccagatcgagaccctgctgggttttgtctataacatcgacggcaacatctggaacgacgcagttactcgcgcgtatctgttcgacgtaccactggccgtcgaatacgtggaacgcgttgctcgtgatggtgtacacccgaaaagctgggcgtttcagcagctggacggtaaaggcgaacgttggctcgcgaaattcggtcaggaatactgcaaaggtctgatccgcttcaaaaaaaaactgaacgacctgctggaaccgtacggtgaaaac

SEQ ID NO: 117 - protein, 423 aa, Artificial sequence
  beta-defensin/D29gp12/TAT47-57 construct 11 Thioredoxin tag
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSGSGAGTDDDDKAMAMNPVSCVRNKGICVPIRCPGSMKQIGTCVGRAVKCCRKKLKMSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 118 - DNA, 1269 bp, Artificial sequence
  beta-defensin/D29gp12/TAT47-57 construct 11 Thioredoxin tag
agcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctgggtcaggatcaggagcaggtaccgacgacgacgacaaggccatggcaatgaacccggtatcttgcgtgcgcaacaaaggtatctgcgttccgattcgttgtccgggttctatgaaacagattggcacttgcgttggtcgtgccgtgaaatgctgccgtaaaaaacttaagatgagcaaaccgtggctgttcaccgtgcatggtactggccagccagatccgctgggtccaggtctgccagctgatactgctcgtgacgtcctggatatctaccgttggcagccgattggtaactacccggctgcagcgttcccaatgtggccgtccgttgaaaaaggcgtagcggaactcatcctgcagatcgaactgaaactggatgcggacccgtatgctgatttcgcgatggctggctactctcagggtgctatcgtagtaggccaggtcctgaaacatcacatcctgccaccgactggtcgtctgcaccgtttcctgcaccgcctgaaaaaagtgatcttctggggtaacccgatgcgtcagaaaggctttgcacactccgacgagtggattcacccagttgctgcaccggatactctgggcatcctggaagatcgcctggagaacctggaacagtacggtttcgaagtgcgtgactatgcccacgacggtgatatgtacgcctccatcaaagaggacgatctgcacgagtacgaggtagcgatcggtcgtatcgttatgaaagcgagcggtttcattggcggccgtgattctgtagtggcgcagctgatcgaactgggtcagcgcccgattacggaaggtattgcactggcaggcgcgattattgacgccctgaccttctttgcccgctctcgtatgggcgacaaatggccacacctgtataaccgctacccagcagttgagtttctgcgtcagatc

SEQ ID NO: 119 - DNA, 20 bp, Artificial sequence
  primer 27f (190)
agagtttgatcctggctcag

SEQ ID NO: 120 - DNA, 22 bp, Artificial sequence
  primer 1492r (191)
tacggttaccttgttacgactt